SEQUENCE LISTING (73 sequences)

SEQ ID NO: 1 - DNA, 18 bp, Artificial Sequence
  Primer
gatcnngatcntcngang

SEQ ID NO: 2 - DNA, 18 bp, Artificial Sequence
  Primer
gatcnngatcntcngang

SEQ ID NO: 3 - DNA, 18 bp, Artificial Sequence
  Primer
gatcnngatcntcngang

SEQ ID NO: 4 - DNA, 74 bp, Artificial Sequence
  Primer
gaattctaatacgactcactatagggttaactttaagaaggagatatacatatgatggtccagctnctcgagtc

SEQ ID NO: 5 - DNA, 53 bp, Artificial Sequence
  Primer
taatacgactcactatagggaagcttggccaccatggtccagctnctcgagtc

SEQ ID NO: 6 - DNA, 34 bp, Artificial Sequence
  Oligonucleotide SfilNotIFor
catggcggcccagccggcctaatgagcggccgca

SEQ ID NO: 7 - DNA, 34 bp, Artificial Sequence
  Oligonucleotide SfilNotIRev
agcttgcggccgctcattaggccggctgggccgc

SEQ ID NO: 8 - DNA, 43 bp, Artificial Sequence
  Oligonucleotide HAFor
ctagaatatccgtatgatgtgccggattatgcgaatagcgccg

SEQ ID NO: 9 - DNA, 43 bp, Artificial Sequence
  Oligonucleotide HARev
tcgacggcgctattcgcataatccggcacatcatacggataaa

SEQ ID NO: 10 - DNA, 40 bp, Artificial Sequence
  Oligonucleotide M2For
ctagaagattataaagatgacgacgataaaaatagcgccg

SEQ ID NO: 11 - DNA, 40 bp, Artificial Sequence
  Oligonucleotide M2Rev
tcgacggcgctatttttatcgtcgtcatctttataatcaa

SEQ ID NO: 12 - DNA, 23 bp, Artificial Sequence
  Primer HuVH1aBACK
caggtgcagctggtgcagtctgg

SEQ ID NO: 13 - DNA, 23 bp, Artificial Sequence
  PrimerHuVH2aBACK
cagctcaacttaagggagtctgg

SEQ ID NO: 14 - DNA, 23 bp, Artificial Sequence
  PrimerHuVH3aBACK
gaggtgcagctggtggagtctgg

SEQ ID NO: 15 - DNA, 23 bp, Artificial Sequence
  PrimerHuVH4aBACK
caggtgcagctgcaggagtcggg

SEQ ID NO: 16 - DNA, 23 bp, Artificial Sequence
  PrimerHuVH5aBACK
gaggtgcagctgttgcagtctgc

SEQ ID NO: 17 - DNA, 23 bp, Artificial Sequence
  PrimerHuVH6aBACK
caggtacagctgcagcagtcagg

SEQ ID NO: 18 - DNA, 24 bp, Artificial Sequence
  PrimerHuJH1-2FOR
tgaggagacggtgaccagggtgcc

SEQ ID NO: 19 - DNA, 24 bp, Artificial Sequence
  Primer HuJH3FOR
tgaagagacggtgaccattgtccc

SEQ ID NO: 20 - DNA, 24 bp, Artificial Sequence
  Primer HuJH4-5FOR
tgaggagacggtgaccagggttcc

SEQ ID NO: 21 - DNA, 24 bp, Artificial Sequence
  Primer HuJH6FOR
tgaggagacggtgaccgtggtccc

SEQ ID NO: 22 - DNA, 23 bp, Artificial Sequence
  Primer HuVkappa1aBACK
gacatccagatgacccagtctcc

SEQ ID NO: 23 - DNA, 23 bp, Artificial Sequence
  Primer HuVkappa2aBACK
gatgttgtgatgactcagtctcc

SEQ ID NO: 24 - DNA, 23 bp, Artificial Sequence
  Primer HuVkappa3aBACK
gaaattgtgttgacgcagtctcc

SEQ ID NO: 25 - DNA, 23 bp, Artificial Sequence
  Primer HuVkappa4aBACK
gacatcgtgatgacccagtctcc

SEQ ID NO: 26 - DNA, 23 bp, Artificial Sequence
  Primer HuVkappa5aBACK
gaaacgacactcacgcagtctcc

SEQ ID NO: 27 - DNA, 23 bp, Artificial Sequence
  Primer HuVkappa6aBACK
gaaattgtgctgactcagtctcc

SEQ ID NO: 28 - DNA, 23 bp, Artificial Sequence
  Primer HuVlambda1BACK
cagtctgtgttgacgcagccgcc

SEQ ID NO: 29 - DNA, 23 bp, Artificial Sequence
  Primer HuVlambda2BACK
cagtctgccctgactcagcctgc

SEQ ID NO: 30 - DNA, 23 bp, Artificial Sequence
  Primer HuVlambda3aBACK
tcctatgtgctgactcagccacc

SEQ ID NO: 31 - DNA, 23 bp, Artificial Sequence
  Primer HuVlambda3bBACK
tcttctgagctgactcaggaccc

SEQ ID NO: 32 - DNA, 23 bp, Artificial Sequence
  Primer HuVlambda4BACK
cacgttatactgactcaaccgcc

SEQ ID NO: 33 - DNA, 23 bp, Artificial Sequence
  Primer HuVlambda5BACK
caggctgtgctcactcagccgtc

SEQ ID NO: 34 - DNA, 23 bp, Artificial Sequence
  Primer HuVlambda6BACK
aattttatgctgactcagcccca

SEQ ID NO: 35 - DNA, 24 bp, Artificial Sequence
  Primer HuJKappa1FOR
acgtttgatttccaccttggtccc

SEQ ID NO: 36 - DNA, 24 bp, Artificial Sequence
  Primer HuJKappa2FOR
acgtttgatctccagcttggtccc

SEQ ID NO: 37 - DNA, 24 bp, Artificial Sequence
  Primer HuJKappa3FOR
acgtttgatatccactttggtccc

SEQ ID NO: 38 - DNA, 24 bp, Artificial Sequence
  Primer HuJKappa4FOR
acgtttgatctccaccttggtccc

SEQ ID NO: 39 - DNA, 24 bp, Artificial Sequence
  Primer HuJKappa5FOR
acgtttaatctccagtcgtgtccc

SEQ ID NO: 40 - DNA, 24 bp, Artificial Sequence
  Primer HuJlambda1FOR
acctaggacggtgaccttggtccc

SEQ ID NO: 41 - DNA, 24 bp, Artificial Sequence
  Primer HuJlambda2-3FOR
acctaggacggtcagcttggtccc

SEQ ID NO: 42 - DNA, 24 bp, Artificial Sequence
  Primer HuJlambda4-5FOR
acctaaaacggtgagctgggtccc

SEQ ID NO: 43 - DNA, 28 bp, Artificial Sequence
  Primer RHuJH1-2
gcaccctggtcaccgtctcctcaggtgg

SEQ ID NO: 44 - DNA, 28 bp, Artificial Sequence
  Primer RHuJH3
ggacaatggtcaccgtctcttcaggtgg

SEQ ID NO: 45 - DNA, 28 bp, Artificial Sequence
  Primer RHuJH3
gaaccctggtcaccgtctcctcaggtgg

SEQ ID NO: 46 - DNA, 28 bp, Artificial Sequence
  Primer RHuJH6
ggaccacggtcaccgtctcctcaggtgg

SEQ ID NO: 47 - DNA, 32 bp, Artificial Sequence
  Primer RHuVkappa1aBACKFv
ggagactgggtcatctggatgtccgattcgcc

SEQ ID NO: 48 - DNA, 32 bp, Artificial Sequence
  Primer RHuVkappa2aBACKFv
ggagactgagtcatcacaacatccgatccgcc

SEQ ID NO: 49 - DNA, 32 bp, Artificial Sequence
  Primer RHuVkappa3aBACKFv
ggagactgcgtcaacacaatttccgatccgcc

SEQ ID NO: 50 - DNA, 32 bp, Artificial Sequence
  Primer RHuVkappa4aBACKFv
ggagactgggtcatcacgatgtccgatccgcc

SEQ ID NO: 51 - DNA, 32 bp, Artificial Sequence
  Primer RHuVkappa5aBACKFv
ggagactgcgtgagtgtcgtttccgatccgcc

SEQ ID NO: 52 - DNA, 32 bp, Artificial Sequence
  Primer RHuVkappa6aBACKFv
ggagactgagtcagcacaatttccgatccgcc

SEQ ID NO: 53 - DNA, 42 bp, Artificial Sequence
  Primer RHuVlambdaBACK1Fv
ggcggctgcgtcaacacagactgcgatccgccaccgccagag

SEQ ID NO: 54 - DNA, 42 bp, Artificial Sequence
  Primer RHuVlambdaBACK2Fv
gcaggctgagtcagagcagactgcgatccgccaccgccagag

SEQ ID NO: 55 - DNA, 42 bp, Artificial Sequence
  Primer RHuVlambdaBACK3aFv
ggtggctgagtcagcacataggacgatccgccaccgccagag

SEQ ID NO: 56 - DNA, 42 bp, Artificial Sequence
  Primer RHuVlambdaBACK3bFv
gggtcctgagtcagctcagaagacgatccgccaccgccagag

SEQ ID NO: 57 - DNA, 42 bp, Artificial Sequence
  Primer RHuVlambdaBACK4Fv
ggcggttgagtcagtataacgtgcgatccgccaccgccagag

SEQ ID NO: 58 - DNA, 42 bp, Artificial Sequence
  Primer RHuVlambdaBACK5Fv
gacggctgagtcagcacagactgcgatccgccaccgccagag

SEQ ID NO: 59 - DNA, 42 bp, Artificial Sequence
  Primer RHuVlambdaBACK6Fv
tggggctgagtcagcataaaattcgatccgccaccgccagag

SEQ ID NO: 60 - DNA, 56 bp, Artificial Sequence
  Primer HuVH1aBACKSfi
gtcctcgcaactgcggcccagccggccatggcccaggtgcagctggtgcagtctgg

SEQ ID NO: 61 - DNA, 56 bp, Artificial Sequence
  Primer HuVH2aBACKSfi
gtcctcgcaactgcggcccagccggccatggcccaggtcaacttaagggagtctgg

SEQ ID NO: 62 - DNA, 56 bp, Artificial Sequence
  PrimerHuVH3aBACKSfi
gtcctcgcaactgcggcccagccggccatggccgaggtgcagctggtggagtctgg

SEQ ID NO: 63 - DNA, 56 bp, Artificial Sequence
  Primer HuVH4aBACKSfi
gtcctcgcaactgcggcccagccggccatggcccaggtgcagctgcaggagtcggg

SEQ ID NO: 64 - DNA, 56 bp, Artificial Sequence
  Primer HuVH5aBACKSfi
gtcctcgcaactgcggcccagccggccatggcccaggtgcagctgttgcagtctgc

SEQ ID NO: 65 - DNA, 56 bp, Artificial Sequence
  Primer HuVH6aBACKSfi
gtcctcgcaactgcggcccagccggccatggcccaggtacagctgcagcagtcagg

SEQ ID NO: 66 - DNA, 48 bp, Artificial Sequence
  Primer HuJKappa1FORNot
gagtcattctcgacttgcggccgcacgtttgatttccaccttggtccc

SEQ ID NO: 67 - DNA, 48 bp, Artificial Sequence
  Primer HuJKappa2FORNot
gagtcattctcgacttgcggccgcacgtttgatctccagcttggtccc

SEQ ID NO: 68 - DNA, 48 bp, Artificial Sequence
  Primer HuJKappa3FORNot
gagtcattctcgacttgcggccgcacgtttgatatccactttggtccc

SEQ ID NO: 69 - DNA, 48 bp, Artificial Sequence
  Primer HuJKappa4FORNot
gagtcattctcgacttgcggccgcacgtttgatctccaccttggtccc

SEQ ID NO: 70 - DNA, 48 bp, Artificial Sequence
  Primer HuJKappa5FORNot
gagtcattctcgacttgcggccgcacgtttaatctccagtcgtgtccc

SEQ ID NO: 71 - DNA, 48 bp, Artificial Sequence
  Primer HuJlambda1FORNot
gagtcattctcgacttgcggccgcacctaggacggtgaccttggtccc

SEQ ID NO: 72 - DNA, 48 bp, Artificial Sequence
  Primer HuJlambda2-3FORNot
gagtcattctcgacttgcggccgcacctaggacggtcagcttggtccc

SEQ ID NO: 73 - DNA, 48 bp, Artificial Sequence
  Primer HuJlambda4-5FORNot
gagtcattctcgacttgcggccgcacctaaaacggtgagctgggtccc

(19) United States
(12) Patent Application Publication
(10) Pub. No.: US 2002/0137053 A1
(43) Pub. Date: Sep. 26, 2002

(54) Collections of binding proteins and tags and uses thereof for nested sorting and high throughput screening

(76) Inventors: Dana Ault-Riche, Palo Alto, CA (US); Paul D. Kassner, San Mateo, CA (US)

Correspondence Address: HELLER EHRMAN WHITE & MCAULIFFE LLP, 4250 EXECUTIVE SQ, 7TH FLOOR, LA JOLLA, CA, 92037, US

(21) Appl. No.: 09/910,120

(22) Filed: Jul. 18, 2001

Related U.S. Application Data
(60) Provisional application No. 60/219,183, filed on Jul. 19, 2000.

(51) Int. Cl. C12Q 1/68; G01N 33/53; G01N 33/542; C12M 1/34

(52) U.S. Cl. 435/6; 435/7.9; 435/287.2

(57) ABSTRACT

Provided herein are addressable collections of anti-tag capture agents, such as antibodies, that are used as tools for sorting proteins containing polypeptide tags for which the capture agents are specific. Also provided are methods of nested sorting using the collections. The methods includes the steps of creating tagged collections of molecules by introducing a set of nucleic acid molecules that encode unique preselected polypeptides to create a library of tagged molecules; either before or after introducing the tags, dividing the library into N divisions; translating each division and reacting each with one of N capture agent collections, identifying the capture agents bound to the polypeptide tags linked to molecules of interest, and thereby identifying the one of the divided collections that contains the molecules of interest. The method can further include adding a new set of tags and repeating the sorting process with the same or a different collection capture agents and thereby identifying a protein or molecule of interest.

### RELATED APPLICATIONS

For U.S. purposes benefit of priority under 35 U.S.C. §119(e) is claimed to U.S. provisional application Serial No. 60/219,183, filed Jul. 19, 2000, to Dana Ault-Riche entitled “COLLECTIONS OF ANTIBODIES FOR NESTED SORTING AND HIGH THROUGHPUT SCREENING”. For international purposes priority is claimed to U.S. provisional application Serial No. 60/219,183. Where permitted, the subject matter of U.S. provisional application Serial No. 60/219,183 is incorporated in its entirety by reference thereto.

### FIELD OF INVENTION

The present invention relates to collections of binding proteins, called capture agents herein, and methods of use thereof for functional surveys of large diversity libraries, including gene libraries. The methods and collection technology integrate robotic micro-well high throughput screening and array and related techniques.

### BACKGROUND OF THE INVENTION

Genomics and Proteomics

The Human Genome Project has generated an avalanche of genomic data. Unraveling this data will increasee the understanding of biology and ultimately will lead to the development of a new generation of drugs. The availability of gene sequence information is changing the way biomedical research is conducted and the rate of discovery. Having the sequence of a genome, however, does not reveal what the genes do nor how the encoded proteins function, how cells and tissues develop, nor give insights in the etiology and cure of diseases. Before the fruits of the information obtained by sequencing a genome can be realized, encoded proteins and their functions must be identified.

Hence, the emergence of proteomics in which the challenge is to unravel the plethora of information that has been obtained by virtue of sequencing of the human genome and other genomes. The focus is assigning functions to genes that have been identified by sequence. It is, however, a simpler task to identify a gene by sequencing it than it is to discover a function of the gene or the encoded protein. Various approaches, including biochemical, genetic and informatics approaches, to identifying proteins encoded by genes have been pursued in the attempt to do this. Informatics approaches attempt to define gene functions based on computer searches that compare gene sequences with the sequences of genes that encode proteins with known or purportedly known functions. Because of the discontinuity between gene sequence and function, these approaches have had limited success. Defining gene functions remains dependent on traditional approaches of genetics and biochemistry. The genetic approach is based on disrupting a genes function and then observing the effects of that disruption; the biochemical approach is based on correlating biochemical changes with function. To make any headway, high throughput analyses are required.

For genomics, high throughput arrays relying upon hybridization reactions have been employed as a means to identify genes. Proteomics does not as yet have suitable high throughput methodologies. For example, DNA microarrays have been used to determine the amount of messenger RNA (mRNA) for thousands of genes in a given sample. Genes in the DNA are transcribed into mRNA as intermediate molecules before being translated into proteins. The mRNA from two samples are labeled separately by polymerase chain reaction (PCR) amplification with two different dyes, mixed, and then bathed over the array. The PCR products specifically bind to the spots in the array containing nucleic acid that includes complementary sequences of nucleotides. The ratio of dyes, defines the relative amounts of mRNA in the two samples. Computer algorithms are then used to evaluate and interpret the data. Because proteins are central in cellular regulation and because there is a lack of direct correlation between mRNA expression and protein expression, this DNA microarray analysis is inherently limited. The activity of a protein can be modulated by subtle changes in its structure, often as a result of interactions with other proteins or metabolites. Additionally, proteins have differing half-lives and are compartmentalized within the cell. As a result, information about the protein status of a cell, or its “proteome”, in combination with mRNA expression is difficult to obtain.

Protein analysis technologies are based on a combination of protein separation and detection. In two-dimensional (2-D) gel systems, proteins are separated by charge in one dimension and by size in the other. Following separation, proteins are identified by excision from the gel and analysis by mass spectrometry. Although 2-D gel methods can simultaneously analyze over 1,000 proteins, these methods are limited by large sample requirements, poor resolution, low sensitivity, inconsistencies in the results and low throughput.

Protein evolution methods, such as gene shuffling and random saturation mutagenesis by error-prone PCR, link mutation with selection to “evolve” desired traits in proteins thereby providing, for example, a means for creating catalysts for use in industrial processes, for generating new research reagents, and improving the performance of recombinant antibodies. The amount of structural variation possible is enormous. For example, the number of possible combinations for a relatively small protein containing 100 amino acids is 20¹⁰⁰. Additional diversity is provided by including synthetic, or “unnatural”, amino acids. The protein evolution methods can create collections of genes containing trillions of protein variants. Among these trillions are proteins having desirable characteristics. The key to exploiting these diversity-generating methods is the ability to then find the desired “needle” in these very large “haystacks.” This has been attempted using selection methodologies, such as the acquisition of antibiotic resistance, binding to an immobilized capture molecule, and the acquisition of fluorescence followed by particle sorting. Depending on the trait to be evolved, selection schemes are not always possible. Individual testing using high throughput robotic systems are alternatives to selection systems, but these systems become impractical for surveys of greater than half a million clones. None of these methods permits exploitation of the full potential of these diversity-creating methods.

It is apparent that there is a need to identify new methods to sample large diverse collections of proteins and to identify proteins and functions thereof. Therefore, it is an object herein to provide methods and products for identifying desired proteins among large diverse collections of proteins. It is also an object herein to provide products for performing such methods.

### SUMMARY OF THE INVENTION

Provided herein are methods and products for screening and identifying molecules, particularly proteins and nucleic acids, from among large collections. In particular, collections of capture agents (i.e., receptors, such as antibodies or other receptors) that specifically bind to identifiable protein binding partners, designated polypeptide tags herein, in which each capture agent has been selected or designed to bind with high selectivity and specificity to a pre-selected polypeptide tag, such as an epitope or ligand or portion thereof. The collections, which contain indentifiable capture agents, such as antibodies, are provided in any suitable format, including liquid phase and solid phase formats, as long as the capture agents, such as antibodies are identifiable (addressable). Addressable arrays of the capture agents are exemplified herein. The methods herein exemplified with respect to arrays can be practiced with any other format, including capture agents, such as antibodies, linked to RF tags, detectable beads, bar coated beads and other such formats. The collections serve as devices to sort, and ultimately, identify, proteins and genes and other molecules of interest.

The pre-selected polypeptide tags, such as epitope tags, are linked to the molecules, such as proteins, to be sorted. Such linkage can be effected by any means, and is conveniently effected using an amplification scheme or ligation with amplification that incorporates nucleic acids encoding the tags into nucleic acids that encode the proteins to be screened.

Methods of sorting using the protein-tag-labeled collections are provided herein. Hence, provided herein are methods for identification of proteins with desired properties from large, diverse collections of proteins by sorting. Critical to the methods and the addressable collections of binding proteins (capture agents) provided herein is the selection of capture agents, such as antibodies, that bind to a set of pre-selected polypeptide tags of known sequence. The polypeptide tags include a sufficient number of amino acids to specifically binding to the capture agent, such as an antibody. The collections of capture agents, such as antibodies, contain at least about 10, more least about 30, 50, 100, 200, 250, and more, such as at least about 500, 1000, or more, different capture agents, such as antibodies, which bind to different members of the set of polypeptide tags. Methods for producing collections of the capture agents, such as antibodies, are provided herein.

The addressable capture agent, such as antibody, collections provide a means to sort molecules tagged with the sequence of amino acids of the polypeptide that specifically reacts with the capture agent. The sorting relies on the highly specific interaction between capture agents, such as antibodies, in the collection and the polypeptide tags, such as epitope tags, that are introduced into collections of molecules to be sorted.

In one embodiment the addressable capture agents, such as antibodies, are provided as an array, which contains a plurality of capture agents, that are provided on discrete addressable loci on a solid phase. Each address on the array contains capture agents, such as antibodies, that bind to a specific pre-selected tag. Generally all capture agents, such as antibodies, at each locus are identical or substantially identical, but it is only necessary for each agent to have specific high binding affinity (kₐ us generally at least about 10⁻⁷ to 10⁻⁹), to selectively bind to a molecule, generally a protein, that bears the predesigned or preselected polypeptide tag.

In practice proteins tagged with the polypeptide tags are bathed over an array of capture agents or reacted with the collection of capture agents linked to identifiable supports, such as beads, under suitable binding conditions. By virtue of the binding specificity of the preselected tags for particular capture agents, the proteins are sorted according their preselected tag. The identity of the tag and is then known, since it reacts with a particular capture agent whose identity is known by virtue of its position in the array or its identifier, such as its linkage to an optically coded, including as color coded or bar coded, or an electronically-tagged, such as a microwave or radio frequency (RF)-tagged, particle.

In one embodiment, the antibodies are provided in a solid phase format, more preferably organized as an addressable array in which each locus can be identified. Bar codes or other symbologies or indicia of identity may also be included on the solid phase arrays to aid in orientation or positioning of the antibodies. A plurality of such arrays can be included on a single matrix support. In one embodiment, the arrays are arranged and are of a size that matches, for example a 96-well, 384-well, 1536-well or higher density format. In another embodiment, for example, 24 such arrays, with 30 to 1000 antibody loci, such as 30, 100, 200, 250, 500, 750, 1000 or other convenient number, each are in such arrangement. In one embodiment, for example, 96 or more arrays, with 30 to 1000 antibody loci, such as 30, 100, 200, 250, 500, 750, 1000 or other convenient number, each are in such arrangement.

In another embodiment, the solid supports constitute coded particles (beads), such as microspheres that can be handled in liquid phase and then layered into a two dimensional array. The particles, such as microspheres, are encoded by optically, such as by color or bar coded, chemically coded, electronically coded or coded using any suitable code that permits identification of the bead and capture agent bound thereto. The capture agent is coated on or otherwise linked to the support.

The collections of capture agents, such as antibodies, are tools that can be used in a variety of processes, including, but not limited to, rapid identification of antibodies for therapeutics, diagnostics, research reagents, proteomics affinity matrices; enzyme engineering to identify improved catalysts, for antibody affinity maturation, for small molecule capture proteins and sequence-specific DNA binding proteins; for protein interaction mapping; and for development and identification of high affinity T cell receptors (see, e.g., Shusta et al. (2000) Directed evolution of a stable scaffold for T-cell receptor engineering, Nature Biotechnology 18:754-759).

The polypeptide, such as epitope, tags can be introduced into molecules by any suitable methods, including chemical linkage. They can be introduced into proteins by a variety of methods. These include, for example, introduction into nucleic acid encoding the proteins by amplification with primers that encode the tags or by ligation of the oligonucleotides, optionally followed by an amplification, or by cloning into sets of plasmids encoding the tags. For example, the polypeptide, such as epitope, tags are introduced into proteins by amplification, typically PCR, from cDNA libraries using primers that are designed to introduce the tags into the resulting amplified nucleic acid. A plurality of such tags are ultimately introduced into the nucleic acid, to permit sorting upon translation of the nucleic acids and to provide sequences for selective amplification of nucleic acids encoding desired proteins.

The polypeptide tags include a sequence of amino acids (designated “E” herein and for purposes herein generically called epitopes, but including sequence of amino acids to which any capture agent binds), to which the capture agents, such as antibodies, are designed or selected to bind. The E portion (as noted generally referred to herein as an epitope, but not limited to sequences of amino acids that bind to antibodies) of the tag includes a sufficient number of amino acids to selectively bind to a capture agent. It also, in certain embodiments, includes a sequence referred to herein as a divider (D), which includes one or more amino acids, typically, at least three amino acids, and generally includes 4 to 6 amino acids. The epitope and divider sequences can include more amino acids and additional regions, as needed, for amplification of DNA encoding such tags or for other purposes. As noted below, the polypeptide tag may also include a region designated “C.”

Methods using the capture agent (also referred to herein as a receptor) collections, such as antibody collections, for sorting molecules labeled with the binding pair, such as an epitope, tags are provided. The methods include the steps of creating a master tagged library by adding nucleic acids encoding the tags; dividing a portion of the master library into N reactions; amplifing each reaction with the nucleic acid encoding the divider sequences and translating to produce N translated reactions mixtures; reacting each of the reactions mixtures with one collection of the antibodies, using for example conditions used for western blotting; identifying the proteins of interest by a suitable screen, thereby identifying the particular polypeptide tag on the protein by virtue of the capture agent which the protein of interest binds.

The first sort is designed to reduce diversity by a significant factor. Standard screening methods may then be employed to screen the new sublibrary. If a further reduction is diversity is desired a second sort can be performed. By appropriate selection of the number of antibodies (or other receptors), the number of D's and pools and the number of collections in the first screen, the optional second screen can be designed so that the resulting collection should contain only a single protein or only a small number of proteins.

A second sort starting from the nucleic acid reaction mixture reaction that contains the nucleic acid from which the protein of interest was translated can be performed performed. In this step, a new set of the polypeptide tags is added to the nucleic acid by amplification or ligation followed by amplification. Prior to or simultaneously with this, the nucleic acid encoding the prior polypeptide tag, such as epitope tag, is removed either by cleavage, such as with a restriction enzyme or by amplification with a primer that destroys part or all of the epitope-encoding nucleic acid. The new tags are added, resulting nucleic acids are translated and are reacted with a single addressable collection of antibodies. The proteins sort according to their polypeptide tag, and a screen is run to identify the protein of interest. At this point, the diversity of the molecules at the addressable locus of the antibody collection should be 1 (or on the order of 1 to 10). The nucleic acids that contain the protein of interest are then amplified with a tag that amplifies nucleic acid molecules that contain nucleic acids encoding the identified polypeptide tag, to thereby produce nucleic acid encoding a protein of interest. The primer for amplification, particularly in methods in which a second or additional sorting steps are contemplate, can include all or only a sufficient portion of the tag to serve as a primer to thereby remove at least part of the “E” portion of the polyeptide tag from the encoded protein.

For a particular sorting step (step i), there are Mⁱ polypeptide tags, designated E₁-Eₘ, which are equal to the number of different capture agents, such as antibodies in the collection, and Nⁱ divider regions, where N is the number of samples that are amplified by each individual divider region, and “i”, which is at least 1, refers to the sorting step. At each sorting step, the number of tags and divider regions may be different. Hence there are N divider regions, designated D₁-Dₙ. N is also the number of replicate arrays or collections used in the first step in the sorting process. The first step in the process reduces the diversity by a particular amount depending upon the initial diversity and M and N.

In exemplified embodiments, the master libraries are complementary DNA (cDNA) libraries and the polypeptide tags are encoded by primers or oligonucleotides that are introduced into the cDNA molecules in the library. In the first step in these methods, a master collection of nucleic acids, which each include, generally at one end, such as at the 3′-end or 5′-end of the nucleic acid molecule, nucleic acid encoding a preselected polypeptide containing an epitope (i.e., specific sequence of amino acids required for specific binding to the capture agent), is prepared. Samples from the master collection are divided into N pools, such as 50, 100, 200, 250 (or conveniently 96 or a multiple (96, 96×1, 96×2 . . . n, wherein n is 1 to as many pools as needed, such as 10, 20, 30, 40, 50, 60, 70, 80, 90, 100, 150, 200, 300, 500, 10^{r}, where r is 2 or more, thereof). In each pool one of the n divider sequences (Dₙ) is used to amplify all nucleic acids that include that particular D.

Each amplified pool is translated and the proteins contained therein are contacted with one of the cature agent collections, such as antibody collections, in which the tag for which each capture agent is specific and is known, such as by virtue of its position in an addressable two or three-dimensional array or its linkage to an identifiable particulate support. After contacting, capture agent-protein complexes are identified using standard methods, such as an assay specific for the protein(s) of interest, or by addition of other suitable reagents. Colorimetric, luminescent, fluorescent and other such assays are among the screening assays contemplated. By identifying the capture agent, i.e., antibody, to which the protein of interest binds and the pool containing such capture agent, the original Dₙ pool is known as well as the epitope in the pool and diversity is reduced by n×m. A set of primers containing a portion of the epitope, designated FA, and including all of the E's, is used to amplify the Dₘ pool. This specifically amplifies only members of the pool that include the identified E tag, destroys the epitope in the translated protein and introduces a new set of polypeptide tags encoding nucleic acid molecules into the pool, which is then translated and contacted with a single collection of antibodies; the collection is screened to identify complexes. Amplification of the nucleic acid encoding the identified E tag with a primer contain FB, where FB is all or a portion of the epitope, followed by translation results in a sample containing the protein(s) of interest.

If further reduction in diversity is desired, additional sorting steps may be employed using Mᵢ and Nᵢ tags, where “i” refers to the sorting step number and signifies that M and N may be different at each step. Each M and N can be selected to achieve the desired reduction in diversity. The diversity of the library=Div, is the number of different genes or proteins in a library, Nᵢ is the number of divider sequences (each divider sequence is designated Dₙ used in a particular sorting step, wherein n is from 2 up to N, typically at least about 10 to Nᵢ×Mᵢ, is the number of polypeptide tags, Mᵢ is the number of different capture agents, such as antibodies and/or other receptors or portions thereof, in a collection, and each polypeptide tag is designated Eₘ, where m is 2 to Mᵢ, preferably at least about 10 to M, and i is from 1 to Q, and Q is the number of sorting steps with the antibody collection. In particular, the diversity of the library (Div), Div=(Nᵢ×Mᵢ)(Nᵢ₊₁×M₁₊₁) . . . (N_{Q}×M_{Q}) where i, the sorting step is 1 to Q. If N, Nᵢ . . . N_{Q} are the same number at each step, and M, Mᵢ . . . M_{Q} are the same number at each step, the DIV=(N×M)^{Q}. If the goal is to reduce diversity to a desired level, such as 1, then Div/(Nᵢ×Mᵢ)(Nᵢ₋₁×Mᵢ₋₁) . . . (N_{Q}×M_{Q})=the desired level of diversity, and M and N at each sort should be selected accordingly.

Hence, for example, if there are 10⁶ proteins in a library, if there there are 100 different antibodies in each collection (M), and 100 replicate antibody collections are used (N), and there are two (Q=2) sorting steps, then for a library with a diversity of 10⁶ (Div), the number of reactions into which the initial master collection is divided, will be 100. Generally the number of sorts is one or two. It can be more, but the last step is designed so that at this step substantially all of the molecules at a locus are the same. Alternatively, there may be fewer sorting steps, typically one, which substantially reduce the diversity. Other screening methods can be used in place of further sorting steps to identify proteins corresponding to library members of interst. In this example, after the first sort, the diversity is reduced such that a protein corresponding to library member of interest is present at about 1 in 100; diversity (DIV) has been reduced by a factor of 10⁴. Rather than perform a second sort, other screening methodologies can be used to identify the desired one amongst 100.

Methods for selecting and preparing the capture agent, such as antibody, members of the collections are also provided. Methods for designing polypeptide tags and for preparing antibodies that specifically bind to the tags are provided. Methods for preparing primers and sets of primers are also provided.

Oligonucleotides and sets thereof for introducing the tags for performing the sorting processes are also provided. Sets of oligonucleotides, which are single-stranded for embodiments in which they are used as primers or double-stranded (or partially double-stranded) for embodiments in which they are introduced by ligation for preparation of tagged proteins are also provided. Methods for designing the primers are also provided.

Combinations of an array or set of beads (i.e., particulate supports) linked or coated with capture agents, such as anti-tag antibodies, and the polypeptide tags to which the capture agents specifically bind or a set of expression vectors encoding the polypeptide tags are provided. The vectors optionally contain a multiple cloning site for insertion of a cDNA library of interest. The combinations may further include enzymes and buffers that are necessary for the subcloning, and competent cells for transformation of the library and oligonucleotide primers to use for recovery of the sublibrary of interest. Also provided are combinations containing two or more of the array or set of beads coated with or linked to the capture agents, such as anti-tag antibodies, a set of oligonucleotides encoding the polypeptide tags, any common regions necessary for appending to a cDNA library of interest, and optionally any enzymes and buffers that are used in the ligation, ligase chain reaction (LCR), polymerase chain reaction (PCR), and/or recombination necessary for appending the panel of tags to the cDNA in a library. The combinations may further include a system for in vitro transcription and translation of the protein products of the tagged cDNA, and optionally oligonucleotide primers to use for recovery of the sublibrary of interest. Kits containing these combinations suitably packaged for use in a laboratory and optionally containing instructions for use are also provided.

In one embodiment, combinations of the collections of capture agents, such as antibodies and oligonucleotides that encode polypeptide epitopes to which the capture agents selectively bind are provided. Kits containing the oligonucleotides and capture agents, such as antibodies, and optionally containing instructions and/or additional reagents are provided. The combinations include a collection of capture agents, antibodies, that specificatily bind to a set of preselected epitopes, and a set of oligonucleotides that encode each of the epitopes. The oligonucleotides are single-stranded, double-stranded or include double-stranded and single-stranded portions, such as single-stranded overhangs created by restriction endonuclease cleavage.

### DESCRIPTION OF THE DRAWINGS

FIG. 1 illustrates the concept of nested sorting.

FIG. 2 also illustrates nested sorting; this sort is identical to the sort illustrated in FIG. 1 except that the F2 and F3 sublibrarys have been arranged into arrays.

FIG. 3 illustrates the use antibody arrays as a tool for nested sorts of high diversity gene libraries.

FIG. 4 illustrates application of the methods provided herein for searching libraries of mutated genes.

FIG. 5 illustrates a method for constructing recombinant antibody libraries.

FIG. 6 depicts one method for incorporating polypeptide (epitope) tags into recombinant antibodies using primer addition.

FIG. 7 depicts an altenative scheme using linker addition.

FIG. 8 depicts application of the methods herein for searching recombinant antibody libraries.

FIG. 9 schematically depicts elements of the primers provided herein and the sets of primers required.

FIGS. 10 and 11 depict alternative methods for constructing the ED and EDC primers; in FIG. 10 oligonucleotides are chemically synthesized 3′ to 5′ on a solid support; in the method in FIG. 11, the oligonucleotides self-assemble based upon overlapping hybridization.

FIG. 12 depicts a high throughput screen for discovering immunoglobulin (Ig) produced from hybridoma cells for use in the arrays.

FIGS. 13 (13A and 13B) depict exemplary primers (see SEQ ID Nos. 12-73) for amplification of antibody chains for preparation of recombinant human antibodies (see Table 33, pages 87-88 in McCafferty et al. (1996) Antibody engineering: A practical Approach, Oxford University Press, Oxford, see also, Marks et al. (1992) Bio/Technology 10:779-783; and Kay et al. (1996) Phage Display of Peptides and Proteins: A Laboratory Manual, Academic Press, San Diego).

FIGS. 14 (A-D) depict use of the methods herein for antibody engineering.

FIG. 15 depicts use of the methods herein for identification of antibodies with modified specificity (or any protein with modified specificity).

FIG. 16 depicts use of the methods herein for simultaneous antibody searches.

FIG. 17 depicts use of the methods herein in enzyme engineering protocols

FIG. 18 depicts use of the methods herein in protein interaction mapping protocols.

FIG. 19 depicts the rate of and increase in the number of tags when multiple polypeptide tags are used for sorting.

For clarity of disclosure, and not by way of limitation, the detailed description is divided into the subsections that follow.

### DETAILED DESCRIPTION

A. Definitions

Unless defined otherwise, all technical and scientific terms used herein have the same meaning as is commonly understood by one of skill in the art to which this invention belongs. In the event there are different definintions for terms herein, the definitions in this section control. Where permitted, all patents, applications, published applications and other publications and sequences from GenBank and other databases referred to throughout in the disclosure herein are incorporated by reference in their entirety.

As used herein, nested sorting refers to the process of decreasing diversity using the addressable collections of antibodies provided herein.

As used herein, an addressable collection of anti-tag capture agents (also referred to herein as an addressable collection of capture agents) protein agents (i.e., receptors), such as antibodies, that specifically bind to pre-selected polypeptide tags that contain epitopes (sequences of amino acids, such as epitopes in antigens) in which each member of the collection is labeled and/or is positionally located to permit identification of the capture agent, such as the antibody, and tag. The addressable collection is typically an array or other codable collection in which each locus contains receptors, such as antibodies, of a single specificity and is identifiable. The collection can be in the liquid phase if other discrete identifiers, such as chemical, electronic, colored, fluorescent or other tags are included. Capture agents, include antibodies and other anti-tag receptors. Any protein that specifically binds to a pre-determined sequence of amino acids, such as an epitope, is contemplated for use as a capture agent.

As used herein, polypeptide tags, herein to generically refer to the tags include a sequence of amino acids, that specifically binds to a capture agent.

As used herein, an epitope tag refers to a sequence of amino acids that includes the sequence of amino acids, herein referred to as epitope, to which an anti-tag capture agent, such as an antibody specifically binds. For polypeptide and epitope tags, the specific sequence of amino acids to which each binds is referred to herein generically as an epitope. Any any sequence of amino acids that binds to a receptor therefor is contemplated. For purposes herein the sequence of amino acids of the tag, such as epitope portion of the epitope tag, that specifically binds to the capture agent is designated “E”, and each uniquie epitope is an Eₘ. Depending upon the context “Eₘ” can also refer to the sequences of nucleic acids encoding the amino acids constituting the epitope. The polypeptide tag, such as epitope tag, may also include amino acids that are encoded by the divider region. In particular, the epitope tag is encoded by the oligonucleotides provided herein, which are used to introduce the tag. When reference is made to an epitope tag (i.e. binding pair for a particular receptor or portion thereof) with respect to a nucleic acid, it is nucleic acid encoding the tag to which reference is made. For simplicity each polypeptide ag is referred to as Eₘ; when nucleic acids are being described the Eₘ is nucleic acid and refers to the sequence of nucleic acids that encode the epitope; when the translated proteins are described Em refers to amino acids (the actual epitope). The number of E's corresponds to the number of antibodies in an addressable collection. “m” is typically at least 10, more preferably 30 or more, more preferably 50 or 100 or more, and can be as high as desired and as is practical. Most preferably “m” is about a 1000 or more.

As used herein, Dₙ refers to each divider sequence. As described herein in certain embodiments in which division is effected by other methods Dₙ is optional. As with each Eₘ the Dₙ is either nucleic acid or amino acids depending upon the context. Each Dₙ is a divider sequence that is encoded by an nucleic aicd that serves as a priming site to amplify a subset of nucleic acids. The resulting amplified subset of nucleic acids conains all of the collection of Em sequences and the Dₙ sequences used as a priming site for the amplification. As described herein, the nucleic acids include a portion, preferably at the end, that encodes each EₘDₙ. Generally the encoding nucleic acid is 5′-Eₘ-Dₙ-3′ on the nucleic acid molecules in the library). D is an optional unique sequence of nucleotides for specific amplification to create the sublibrarys. For large libraries, the original library can be divided into sublibraries and then the tag-encoding seuqences added, rather than adding the tag-encoding sequences to the master library, The size of D is a function of the library to be sorted, since the larger the library the longer the sequence neeeded to specify a unique sequence in the library. Generally D, dependening upon the application, should be at least 14 to 16 nucleic acid bases long and it may or may not encoded a sequence of amino acids, since its function in the method is to serve as a priming site for PCTR amplification, D is 2 to n, where n is 0 or is any desired number and is generally 10 to 10,000, 10 to 1000, 50 to 500, and about 100 to 250. The number of D can be as high as 10⁶ or higher. The divider sequences D are used to amplify each of the “n” samples from the tagged master library, and generally is equal to the number of antibody collections, such as arrays, used in the initial sort. The more collections (divisions) in the initial screen, the lower diversity per addressable locus. The initial division number is selected based upon the diverity of the library and the number of capture agents. The more E's, the fewer D's are needed, and vice versa, for a library having a particular diversity (Div). As used herein, diversity (Div) refers to the number of different molecules in a library, such as a nucleic acid library. Diversity is distinct from the total number of molecules in any library, which is greater. The greater the diversity, the lower the number of actual duplicates there are. Ideally the (number of different molecules)/(total molecules) is approximately 1. If the number of molecules that are randomly tagged to create the master library, is less than the initial diversity, then statistically each of the molecules in the master library should be different.

As used herein, an array refers to a collection of elements, such as antibodies, containing three or more members. An addressable array is one in which the members of the array are identifiable, typically by position on a solid phase support or by virtue of an identifiable or detectable label, such as by color, fluorescence, electronic signal (i.e. RF, microwave or other frequency that does not substantially alter the interation of the molecules of interest), bar code or other symbology, chemical or other such label. Hence, in general the members of the array are immobilized to discrete identifiable loci on the surface of a solid phase or directly or indirectly linked to or otherwise associated with the identifiable label, such as affixed to a microsphere or other particulate support (herein referred to as beads) and suspended in solution or spread out on a surface.

As used herein, a support (also referred to as a matrix support, a matrix, an insoluble support or solid support) refers to any solid or semisolid or insoluble support to which a molecule of interest, typically a biological molecule, organic molecule or biospecific ligand is linked or contacted. Such materials include any materials that are used as affinity matrices or supports for chemical and biological molecule syntheses and analyses, such as, but are not limited to: polystyrene, polycarbonate, polypropylene, nylon, glass, dextran, chitin, sand, pumice, agarose, polysaccharides, dendrimers, buckyballs, polyacrylamide, silicon, rubber, and other materials used as supports for solid phase syntheses, affinity separations and purifications, hybridization reactions, immunoassays and other such applications. The matrix herein may be particulate or may be a be in the form of a continuous surface, such as a microtiter dish or well, a glass slide, a silicon chip, a nitrocellulose sheet, nylon mesh, or other such materials. When particulate, typically the particles have at least one dimension in the 5-10 mm range or smaller. Such particles, referred collectively herein as “beads”, are often, but not necessarily, spherical. Such reference, however, does not constrain the geometry of the matrix, which may be any shape, including random shapes, needles, fibers, and elongated. Roughly spherical “beads”, particularly microspheres that can be used in the liquid phase, are also contemplated. The “beads” may include additional components, such as magnetic or paramagnetic particles (see, e.g., Dyna beads (Dynal, Oslo, Norway)) for separation using magnets, as long as the additional components do not interfere with the methods and analyses herein.

As used herein, matrix or support particles refers to matrix materials that are in the form of discrete particles. The particles have any shape and dimensions, but typically have at least one dimension that is 100 mm or less, 50 mm or less, 10 mm or less, 1 mm or less, 100 μm or less, 50 μm or less and typically have a size that is 100 mm³ or less, 50 mm³ or less, 10 mm³ or less, and 1 mm³ or less, 100 μm³ or less and may be order of cubic microns. Such particles are collectively called “beads.”

As used herein, a capture agent, which is used interchangeably with a receptor, refers to a molecule that has an affinity for a given ligand or a with a defined sequence of amino acids. Capture agents may be naturally-occurring or synthetic molecules, and include any molecule, including nucleic acids, small organics, proteins and complexes that specifically bind to specific sequences of amino acids. Capture agents are receptors may also be referred to in the art as anti-ligands. As used herein, thee terms, capture agent, receptor and anti-ligand are interchangeable. Capture agents can be used in their unaltered state or as aggregates with other species. They may be attached or in physical contact with, covalently or noncovalently, a binding member, either directly or indirectly via a specific binding substance or linker. Examples of capture agents, include, but are not limited to: antibodies, cell membrane receptors surface receptors and internalizing receptors, monoclonal antibodies and antisera reactive or isolated components thereof with specific antigenic determinants (such as on viruses, cells, or other materials), drugs, polynucleotides, nucleic acids, peptides, cofactors, lectins, sugars, polysaccharides, cells, cellular membranes, and organelles.

Examples of capture agents, include but are not restricted to:

a) enzymes and other catalytic polypeptides, including, but are not limited to, portions thereof to which substrates specifically bind, enzymes modified to retain binding activity lack catalytic activity;

b) antibodies and portions thereof that specifically bind to antigens or sequences of amino acids;

c) nucleic acids;

d) cell surface receptors, opiate receptors and hormone receptors and other receptors that specifically bind to ligands, such as hormones. For the collections herein, the other binding partner, referred to herein as a polypeptide tag for each refers the substrate, antigenic sequence, nucleic acid binding protein, receptor ligand, or binding portion thereof.

As noted, contemplated herein, are pairs of molecules, generally proteins that specifically bind to each other. One member of the pair is a polypeptide that is used as a tag and encoded by nucleic acids linked to the libary; the other member is anything that specifically binds thereto. The collections of capture agents, include receptors, such as antibodies or enzymes or portions thereof and mixtures thereof that specifically bind to a known or knowable defined sequence of amino acids that is typically at least about 3 to 10 amino acids in length.

As used herein, antibody refers to an immuoglobulin, whether natural or partially or wholly synthetically produed, including any derivative thereof that retains the specific binding ability of the antibody. Hence antibody includes any protein having a binding domain that is homologous or substantially homologous to an immunoglobulin binding domain. For purposes herein, antibody includes antibody fragments, such as Fab fragments, which are composed of a light chain and the variable region of a heavy chain Antibodies include members of any immunoglobulin class, including IgG, IgM, IgA, IgD and IgE. Also contemplated herein are receptors that specifically binding to a sequence of amino acids.

Hence for purposes herein, any set of pairs of binding members, referred to generically herein as a capture agent/polypeptide tag, can be used instead of antibodies and epitopes per se. The methods herein rely on the capture agent/polypeptdie tag, such as and antibody/epitope tag, for their specific interactions, any such combination of receptors/ligands (epitope tag) can be used. Furthermore, for purposes herein, the the capture agents, such as antibodies employed, can be binding portions thereof.

As used herein, antibody fragment refers to any derivative of an antibody that is less than full length, retaining at least a portion of the full-lenth antibody's specific binding ability. Examples of antibody fragments include, but are not limited to, Fab, Fab′, F(ab)₂, single-chain Fvs (scFv), Fv, dsFv diabody and Fd fragments. The fragment can include multiple chains linked together, such as by disulfide bridges. An antibody fragment generally contains at least about 50 amino acids and typically at least 200 amino acids.

As used herein, an Fv antibody fragment is composed of one variable heavy domain (V_{H}) and one variable light (V_{L}) domain linked by noncovalent interactions.

As used herein, a dsFv refers to an Fv with an engineered intermolecular disulfide bond, which stablilizes the V_{H}-V_{L} pair.

As used herein, an F(ab)₂ fragment is an antibody fragment that results from digestion of an immunoglobulin with pepsin at pH 4.0-4.5; it may be recombinantly produced.

As used herein, an Fab fragment is an antibody fragment that results from digestion of an immunoglobulin with papain; it may be recombinantly produced.

As used herein, scFvs refer to antibody fragments that contain a variable light chain (V_{L}) and variable heavy chain (V_{H}) covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

As used herein, diabodies are dimeric scFv; diabodies typically have shorter peptide linkers than scFvs, and they preferentially dimerize.

As used herein, humanized antibodies refer to antibodies that are modified to include “human” sequences of amino acids so that administration to a human does not provoke an immune response. Methods for preparation of such antibodies are known. For example, the hybridoma that expresses the monoclonal antibody is altered by recombinant DNA techniques to express an antibody in which the amino acid composition of the non-variable regions is based on human antibodies. Computer programs have been designed to identify such regions.

As used herein, macromolecule refers to any molecule having a molecular weight from the hundreds up to the millions. Macromolecules include peptides, proteins, nucleotides, nucleic acids, and other such molecules that are generally synthesized by biological organisms, but can be prepared synthetically or using recombinant molecular biology methods.

As used herein, the term “biopolymer” is used to mean a biological molecule, including macromolecules, composed of two or more monomeric subunits, or derivatives thereof, which are linked by a bond or a macromolecule. A biopolymer can be, for example, a polynucleotide, a polypeptide, a carbohydrate, or a lipid, or derivatives or combinations thereof, for example, a nucleic acid molecule containing a peptide nucleic acid portion or a glycoprotein, respectively. Biopolymer include, but are not limited to, nucleic acid, proteins, polysaccharides, lipids and other macromolecules. Nucleic acids include DNA, RNA, and fragments thereof. Nucleic acids may be derived from genomic DNA, RNA, mitochondrial nucleic acid, chloroplast nucleic acid and other organelles with separate genetic material.

As used herein, a biomolecule is any compound found in nature, or derivatives thereof. Biomolecules include but are not limited to: oligonucleotides, oligonucleosides, proteins, peptides, amino acids, peptide nucleic acids (PNAs), oligosaccharides and monosaccharides.

As used herein, the term “nucleic acid” refers to single-stranded and/or double-stranded polynucleotides such as deoxyribonucleic acid (DNA), and ribonucleic acid (RNA) as well as analogs or derivatives of either RNA or DNA. Also included in the term “nucleic acid” are analogs of nucleic acids such as peptide nucleic acid (PNA), phosphorothioate DNA, and other such analogs and derivatives or combinations thereof.

As used herein, the term “polynucleotide” refers to an oligomer or polymer containing at least two linked nucleotides or nucleotide derivatives, including a deoxyribonucleic acid (DNA), a ribonucleic acid (RNA), and a DNA or RNA derivative containing, for example, a nucleotide analog or a “backbone” bond other than a phosphodiester bond, for example, a phosphotriester bond, a phosphoramidate bond, a phophorothioate bond, a thioester bond, or a peptide bond (peptide nucleic acid). The term “oligonucleotide” also is used herein essentially synonymously with “polynucleotide,” although those in the art recognize that oligonucleotides, for example, PCR primers, generally are less than about fifty to one hundred nucleotides in length.

Nucleotide analogs contained in a polynucleotide can be, for example, mass modified nucleotides, which allows for mass differentiation of polynucleotides; nucleotides containing a detectable label such as a fluorescent, radioactive, luminescent or chemiluminescent label, which allows for detection of a polynucleotide; or nucleotides containing a reactive group such as biotin or a thiol group, which facilitates immobilization of a polynucleotide to a solid support. A polynucleotide also can contain one or more backbone bonds that are selectively cleavable, for example, chemically, enzymatically or photolytically. For example, a polynucleotide can include one or more deoxyribonucleotides, followed by one or more ribonucleotides, which can be followed by one or more deoxyribonucleotides, such a sequence being cleavable at the ribonucleotide sequence by base hydrolysis. A polynucleotide also can contain one or more bonds that are relatively resistant to cleavage, for example, a chimeric oligonucleotide primer, which can include nucleotides linked by peptide nucleic acid bonds and at least one nucleotide at the 3′ end, which is linked by a phosphodiester bond or other suitable bond, and is capable of being extended by a polymerase. Peptide nucleic acid sequences can be prepared using well known methods (see, for example, Weiler et al., Nucleic acids Res. 25:2792-2799 (1997)).

As used herein, oligonucleotides refer to polymers that include DNA, RNA, nuleic acid anologs, such as PNA, and combinations thereof. For purposes herein, primers and probes are single-stranded oligonucleotides.

As used herein, production by recombinant means by using recombinant DNA methods means the use of the well known methods of molecular biology for expressing proteins encoded by cloned DNA.

As used herein, substantially identical to a product means sufficiently similar so that the property of interest is sufficiently unchanged so that the substantially identical product can be used in place of the product.

As used herein, equivalent, when referring to two sequences of nucleic acids, means that the two sequences in question encode the same sequence of amino acids or equivalent proteins. When “equivalent” is used in referring to two proteins or peptides, it means that the two proteins or peptides have substantially the same amino acid sequence with only conservative amino acid substitutions (see, e.g., Table 1, above) that do not substantially alter the activity or function of the protein or peptide. When “equivalent” refers to a property, the property does not need to be present to the same extent but the activities are preferably substantially the same. “Complementary,” when referring to two nucleotide sequences, means that the two sequences of nucleotides are capable of hybridizing, preferably with less than 25%, more preferably with less than 15%, even more preferably with less than 5%, most preferably with no mismatches between opposed nucleotides. Generally to be considered complementary herein the two molecules hybridize under conditions of high stringency.

As used herein, to hybridize under conditions of a specified stringency is used to describe the stability of hybrids formed between two single-stranded DNA fragments and refers to the conditions of ionic strength and temperature at which such hybrids are washed, following annealing under conditions of stringency less than or equal to that of the washing step. Typically high, medium and low stringency encompass the following conditions or equivalent conditions thereto:

1) high stringency: 0.1× SSPE or SSC, 0.1% SDS, 65° C.

2) medium stringency: 0.2× SSPE or SSC, 0.1% SDS, 50° C.

3) low stringency: 1.0× SSPE or SSC, 0.1% SDS, 50° C. Equivalent conditions refer to conditions that select for substantially the same percentage of mismatch in the resulting hybrids. Additions of ingredients, such as formamide, Ficoll, and Denhardt's solution affect parameters such as the temperature under which the hybridization should be conducted and the rate of the reaction. Thus, hybridization in 5× SSC, in 20% formamide at 42° C. is substantially the same as the conditions recited above hybridization under conditions of low stringency. The recipes for SSPE, SSC and Denhardt's and the preparation of deionized formamide are described, for example, in Sambrook et al. (1989) Molecular Cloning, A Laboratory Manual, Cold Spring Harbor Laboratory Press, Chapter 8; see, Sambrook et al., vol. 3, p. B.13, see, also, numerous catalogs that describe commonly used laboratory solutions). It is understood that equivalent stringencies may be achieved using alternative buffers, salts and temperatures.

The term “substantially” identical or homologous or similar varies with the context as understood by those skilled in the relevant art and generally means at least 70%, preferably means at least 80%, more preferably at least 90%, and most preferably at least 95% identity.

As used herein, a composition refers to any mixture. It may be a solution, a suspension, liquid, powder, a paste, aqueous, non-aqueous or any combination thereof.

As used herein, a combination refers to any association between among two or more items. The combination can be two or more separate items, such as two compositions or two collections, can be a mixture thereof, such as a single mixture of the two or more items, or any variation thereof.

As used herein, fluid refers to any composition that can flow. Fluids thus encompass compositions that are in the form of semi-solids, pastes, solutions, aqueous mixtures, gels, lotions, creams and other such compositions. As used herein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Bejacmin/Cummings Pub. co., p.224).

Such substitutions are preferably made in accordance with those set forth in TABLE 1 as follows:

TABLE 1

| Original residue | Conservative substitution |
|---|---|
| Ala (A) | Gly; Ser |
| Arg (R) | Lys |
| Asn (N) | Gln; His |
| Cys (C) | Ser |
| Gln (Q) | Asn |
| Glu (E) | Asp |
| Gly (G) | Ala; Pro |
| His (H) | Asn; Gln |
| Ile (I) | Leu; Val |
| Leu (L) | Ile; Val |
| Lys (K) | Arg; Gln; Glu |
| Met (M) | Leu; Tyr; Ile |
| Phe (F) | Met; Leu; Tyr |
| Ser (S) | Thr |
| Thr (T) | Ser |
| Trp (W) | Tyr |
| Tyr (Y) | Trp; Phe |
| Val (V) | Ile; Leu |

Other substitutions are also permissible and may be determined empirically or in accord with known conservative substitutions.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:1726).

The methods and collections herein are described and exemplified with particular reference to antibody capture agents, and polypeptide tags that include epitopes to which the antibodies bind, but is it to be understood that the methods herein can be practiced with any capture agent and any polypeptide tag therefor. It also to be understood that combinations of collections of any capture agents and polypeptide tag therefor are contemplated for use in any of the embodiments described herein. It is also to be understood that reference to array is intended to encompass any addresable collection, whether it is in the form of a physical array or labeled collection, such as capture agents bound to colored beads.

B. Design and Preparation of Oligonucleotides/Primers

Sorting large diversity libraries onto arrays and amplifying specific pools containing clones with the desired properties is dependent on the ability to uniquely tag a library with specific polypeptide tags. Oligonucleotide sets are chemically synthesized, randomly combined by overlapping sequences, and ligated together to produce a template for enzymatic synthesis of the collection of primers or linkers.

The oligonucleotides are either single-stranded or double-stranded depending upon the manner in which they are to be incorporated into the master library. For example, they can be incorporated, for example by ligation of the double stranded version, such as through a convenient restriction site, followed by amplification with a common region, or they can be incorporated by PCR amplification, in which case the oligonucleotides are single-stranded.

1. Primers

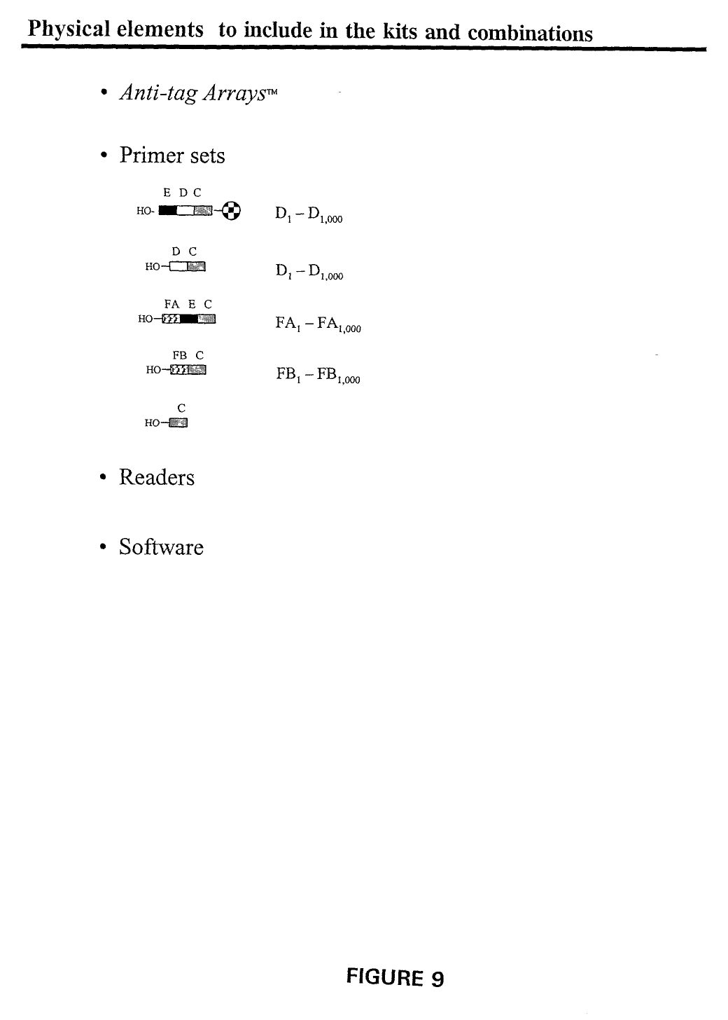

Provided herein are sets of nucleic acid molecules that are primers or double-stranded oligonucleotides, which are double-stranded versions of the primers, and combinations of sets of primers and/or double-stranded oligonucleotides. The selection of single-stranded or double-stranded primers the use in the various steps of the methods provided herein and/or depends upon the embodiment employed. The primers, which are employed in some of the embodiments of the methods for tagging molecules, are central to the practice of such methods. The primers contain oligonucleotides, which include the formulae as depicted in FIG. 9. The primers and double-stranded oligonucleotides may include restriction site(s) and for targeted amplifications, as exemplified below for example for antibody libraries, of sufficient portions of genes of interest. These primers may be forward or reverse primers, where the forward primer is that used for the first round in a PCR ampification. The primers, described below and depicted in the figure, are provided as sets. Also provided are combinations of one or more of each set. The primers are central to the methods provided herein.

2. Preparation of the Oligonucleotides/Primers

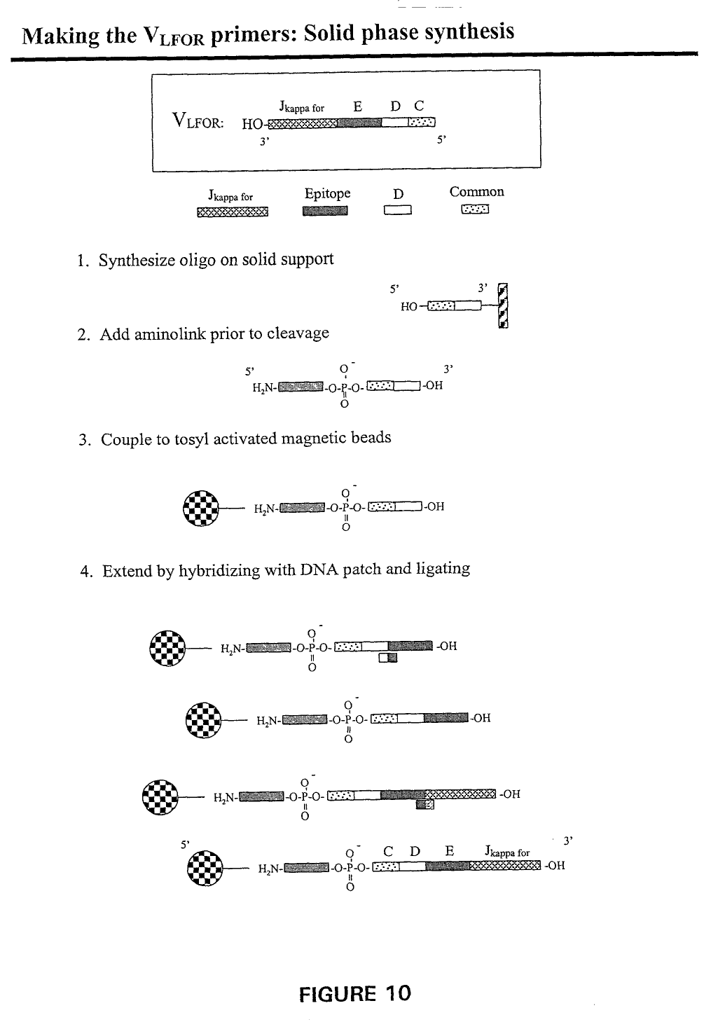

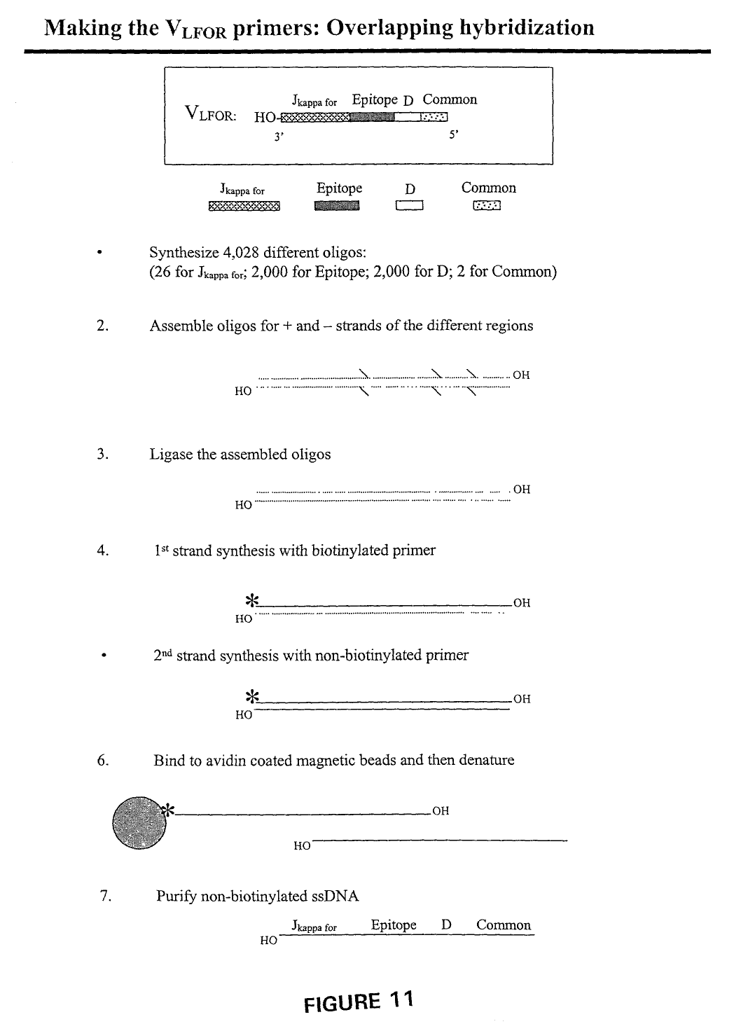

Any suitable method for constructing double-stranded or single-stranded oligonucleotides may be employed. Methods that can be adapted for preparing large numbers of such oligomers are particularly of interest. Two methods are depicted in FIGS. 10 and 11 and are discussed below.

FIG. 9 illustrates the physical elements for construction of a tagged library and use of the addressable anti-tag antibody collections for identification of genes (proteins) of interest. Four oligonucleotide/primer sets are provided in addition to the addressable collections, which for exemplification purposes are provided as arrays, an imaging system or reader to analyze the arrays and, optionally software to manage the information collected by the reader. In the embodiment depicted, the primer sets include EₘDₙC, where C is a portion in common amongst all of the oligonucleotides and can serve as a region for amplification of all tagged nucleic acids with differing E and/or D sequences (e.g., D₁ thru Dₙ; E₁ thru Eₘ); DC, with differing D sequences (D₁ thru Dₙ), and an opptional C, for common region, FAEC, with differing FA sequences (e.g., FA₁ thru FAₙ); and FBC, with differing FB sequences (e.g., FB₁ thru FBₙ). Each FA includes a portion of each epitope and can serve as a primer to amplify nucleic acids that encode a corresponding Eₘ, but the resulting amplified nucleic acids does not include the Eₘ epitope. FBₙ is similar to FAₙ, except that it can include Eₙ, if it is desired to retain the epitope.

FIG. 10 and FIG. 11 outline two different methods for constructing the ED, and EDC, FA and FB oligonucleotides/primers for antibody screening as an example. For example, synthesis of the V_{LFOR} primer, which combines n, such as a 1,000, different E sequences with m, such as 1,000 different D sequences and approximately 13 different Jₖₐₚₚₐ For sequences. This makes a total of (1,000)(1,000)(13) =13,000,000 different oligonucleotides. By randomly combining the different sequence regions in progressive synthesis steps, this large diverse collection of primers can be prepared.

The first method (FIG. 10) uses a solid-phase synthesis strategy. The second method (FIG. 11) uses the ability of DNA molecules to self-assemble based on overlapping complementary sequences. Solid-phase synthesis has the advantage that the immobilized product molecules can be easily purified from substrate molecules between reactions, allowing for greater control of the reaction conditions. The self assembly method has the advantage of requiring much less work.

FIG. 10 Oligonucleotides are chemically synthesized 3′ to 5′ from a solid support. In contrast, DNA is enzymatically synthesized 5′ to 3″. To create the V_{LFOR} primer, the C and D sequences are chemically synthesized using standard methods from a solid support. In order to couple the oligonucleotide to a solid-phase for further synthesis, a strong nucleophile is incorporated by addition of an aminolink prior to cleavage of the oligonucleotide from its substrate. The aminolink introduces a primary amine to the 5′ end of the oligonucleotide. The amine group on the aminolink can then be coupled to a solid support, such as paramagnetic beads, by reaction with amine reactive groups on the beads, such as tosyl, N-hydroxysuccinimide or hydrazine groups. The resulting oligonucleotides are covalently coupled to the beads with the C and D sequences in the proper 5′ to 3′ orientation.

A mixture of E sequences are added to the oligonucleotide by use of a DNA “patch” and the resulting nick is sealed with DNA ligase. Unincorporated substrate DNA is purified from the extended product and a mixture of Jₖₐₚₚₐ for sequences are added to the primer. Although the completed V_{LFOR} primer can be released from the bead, the beads do not interfere with the ability of oligonucleotides to prime cDNA synthesis.

The method illustrated in FIG. 11 relies on the oligonucleotides to self-assemble based on overlapping hybridization. A double stranded DNA molecule is first created from oligonucleotides encoding the +and − strands of the molecule. These oligonucleotides are combined and allowed to hybridize to produce a nicked double-stranded DNA molecule and the nicks on the molecule are sealed by the addition of DNA ligase. The sealed molecules are used as templates for enzymatic synthesis of a new DNA molecule. DNA synthesis is primed using an oligonucleotide with a group on its 5′ end to allow coupling to a solid support, such as biotin or the aminolink chemistry described above.

Incorporation of the reactive group during enzymatic synthesis enables purification of a single stranded molecule after the synthesis is complete. Although the completed VLFOR primer can be released from the bead, the beads do not interfere with the ability of oligonucleotides to prime cDNA synthesis.

C. Nested Sorting Using Addresable Anti-Tag Receptor Collections

Prior methods for identifying and selecting proteins of interest are hampered by selection biases that are created during successive rounds of enrichment. As provided herein, selection biases can be avoided with the use of identification methods based on sorting rather than selection. These method herein rely upon the use of collections of capture agents, such as a plurality of substantially identical, preferably replicate, collections of agents, such as antibodies, that specifically bind to preselected selected sequences of amino acids (generally at least about 5 to 10, typically at least 7 or 8 amino acids, such as epitopes), that are linked to proteins in a target library or encoded by a target nucleic acid library. Combinations of the capture agents and polypeptide tags that contain the sequence of amino acids to which the capture agent or a binding portion thereof specifically binds are provided. The tags may be linked to members of a nucleic acid library or other library of molecules to be sorted.

1. Overview

The addressable anti-tag capture agent collections, such as an positionally addressable array, contains a collection different capture agetns, such as antibodies that bind to pre-selected and/or pre-designed polypeptide tags, such as epitope tags, with high affinity and specificity. A typical collection contains at least about 30, more prefereably 100, more preferably 500, most preferably at least 1000 capture agents, such as antibodies, that are addressable, such as by occupying a unique locus on an array or by virtue of being bound to bar-coded support, color-coded, or RF-tag labeled support or other such addressable format. Each locus or address contains a single type of capture agent, such as antibody, that binds to a single specific tag. Tagged proteins are contacted with the collection of receptors, such as antibodies in an array, under conditions suitable for complexation with the receptor, such as an antibody, via the epitope tag. As a result, proteins are sorted according to the tag each possesses.

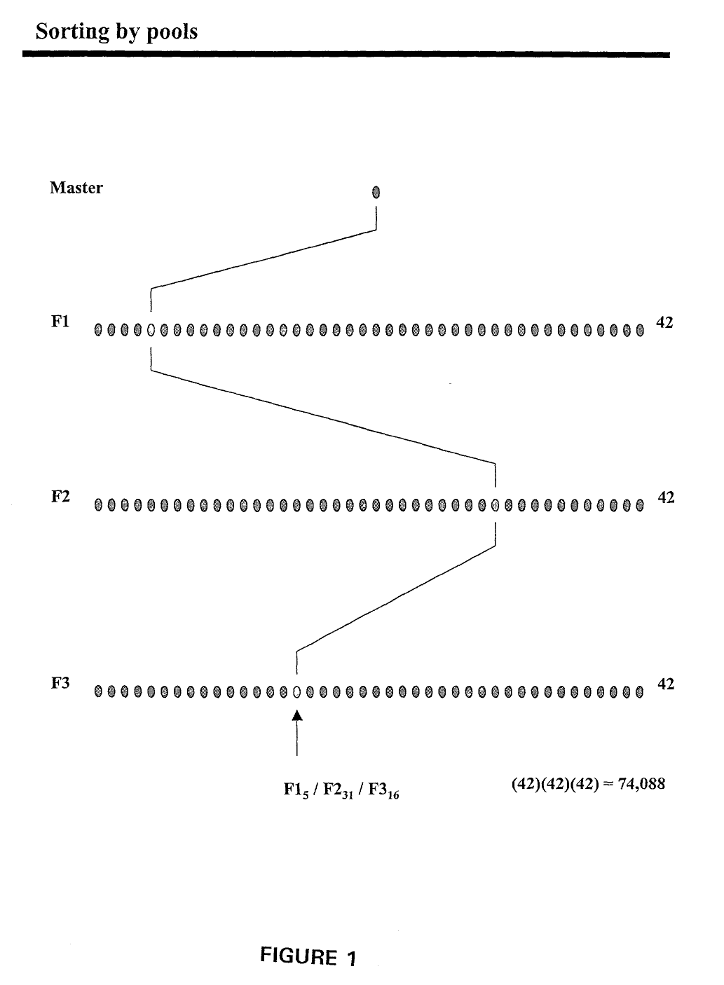

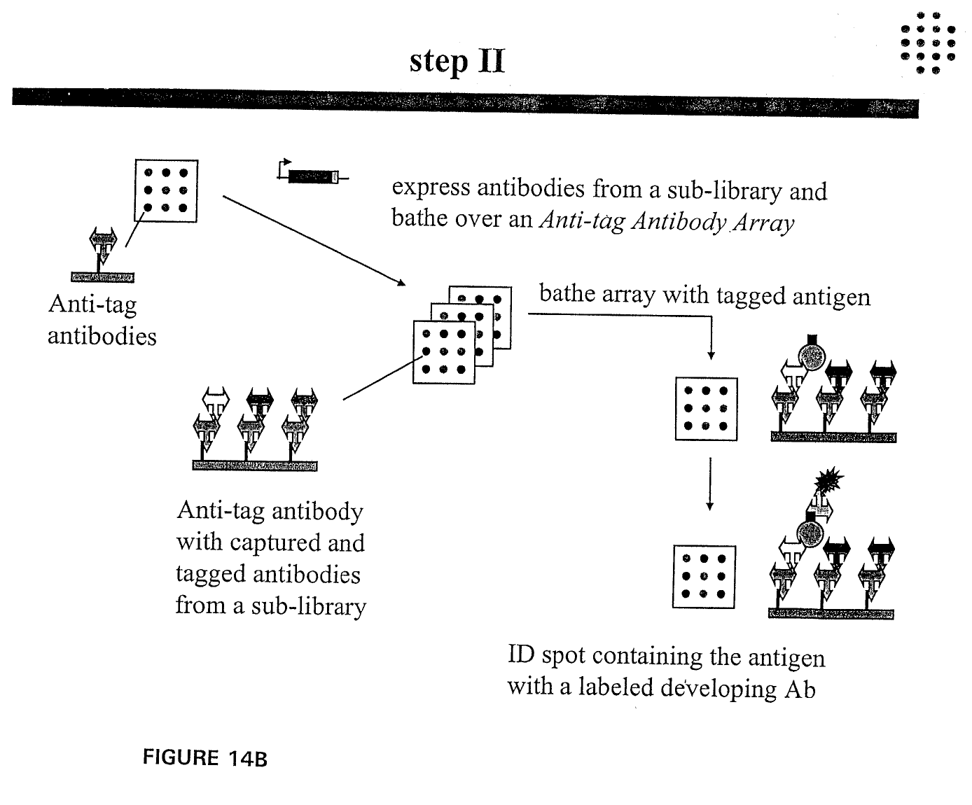

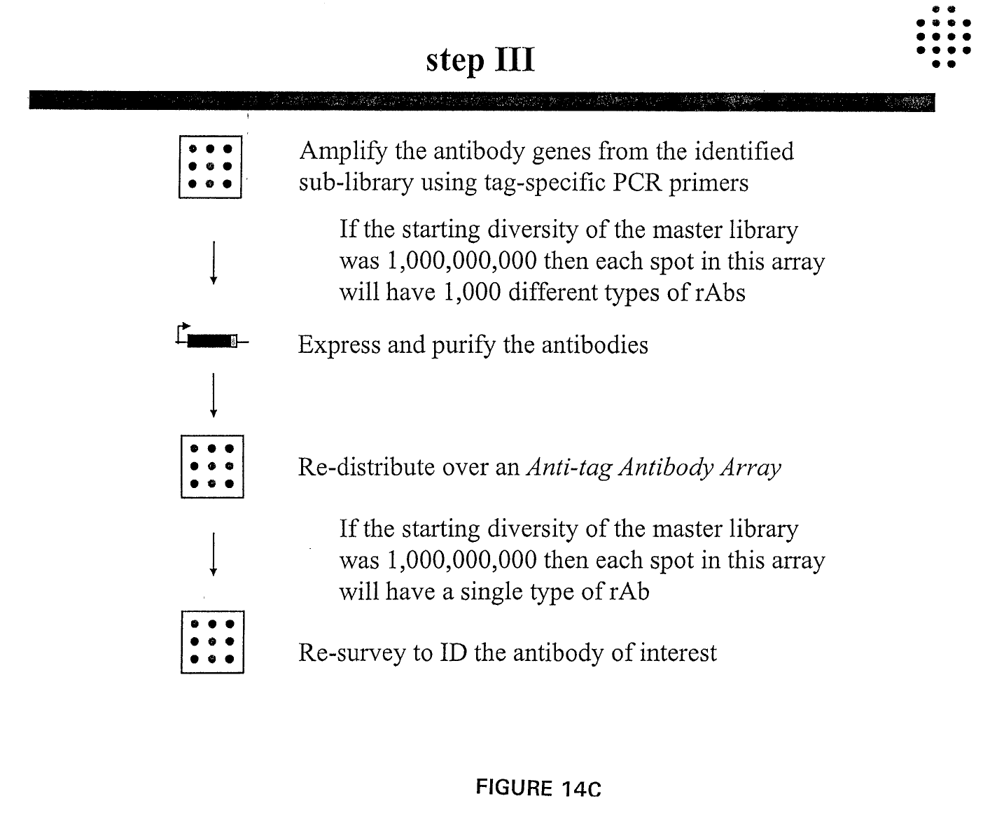

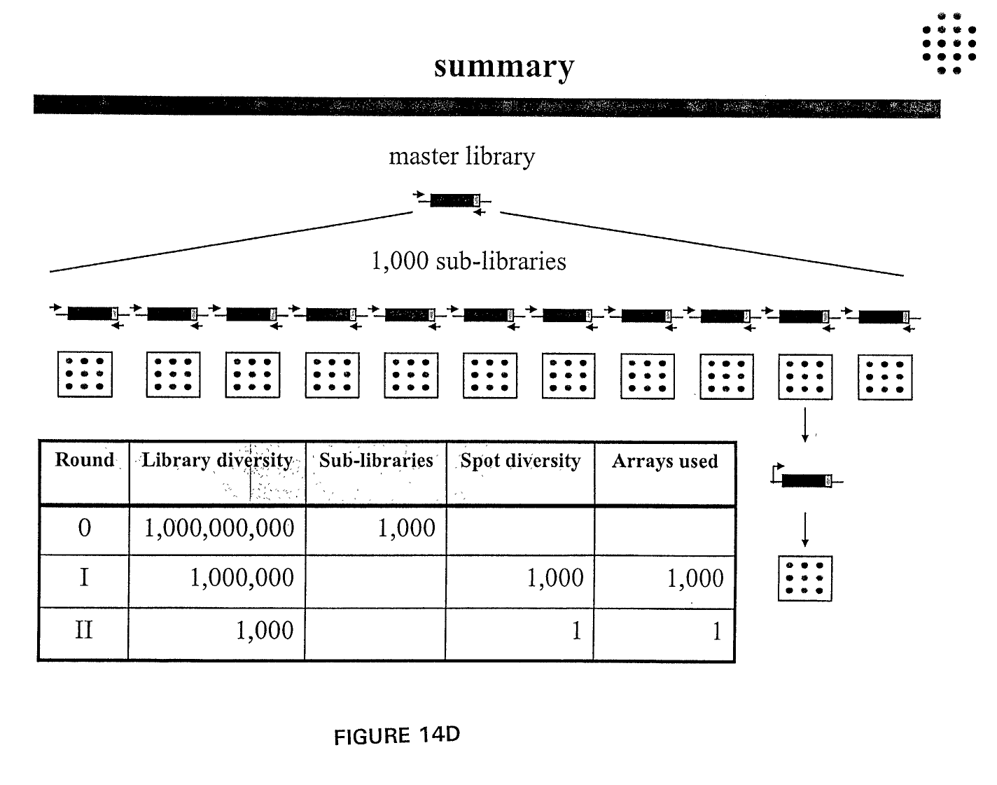

These addressable anti-tag antibody collections have a variety of applications including, but not limited to, rapid identification of antibodies; for therapeutics, diagnostics, reagents, and proteomics affinity matrices; in enzyme engineering applications such as, but not limited to, gene shuffling methodologies; for identification of improved catalysts, for antibody affinity maturation; for identification of small molecule capture proteins, sequence-specific DNA binding proteins, for single chain T-cell receptor binding proteins, and for high affinity molecules that recognize MHC; and for protein interaction mapping. Exemplary protocols are depicted in FIGS. 1-4, 12, 14A-D and 15-18.

2. Sorting Methods

Methods of using the receptor, such as antibody, collections for sorting molecules labeled with the epitope tags are provided. The methods include the steps of creating a master tagged library by adding nucleic acids encoding the tags; dividing a portion of the master library into N reactions; amplifying each reaction with the nucleic acid encoding the divider sequences and translating to produce N translated reactions mixtures; reacting each of the reactions mixtures with one collection of the capture agents, such as antibodies; identifying the proteins of interest by a suitable screen, thereby identifying the particular ED tag on the protein by virtue of the capture agent to which the tag on the protein of interest binds.

The first sorting step substantially reduces diversity. If desired further sorts are performed or the resulting library is sreened by any method known to those of skill in the art. The optional second sort, which is started from the nucleic acid reaction mixture that contains the nucleic acid from which the protein of interest was translated, is performed. In this step, a new set of the epitope tags is added to the nucleic acid by amplification or ligation followed by amplification. Prior to, or simulataneously with this, the nucleic acid encoding the prior epitope tag is removed either by cleavage, such as with a restriction enzyme or by amplification with a primer that destroys part or all of the epitope-encoding nucleic acid. The new tags are added, resulting nucleic acids are translated and are reacted with a single addressable collection of antibodies. The proteins sort according to their polypeptide tag, and a screen is run to identify the protein of interest At this point, the diversity of the molecules at the addressable locus of the antibody collection should be 1 (or on the order of 1 to 100, typically 1 to 10). The nucleic acids that contain the protein of interest are then amplified with a tag that amplifies nucleic acid molecules that contain nucleic acids encoding the identified epitope tag, to thereby produce nucleic acid encoding a protein of interest. The primer for amplificiation includes all or only a sufficient portion of the tag to serve as a primer to thereby removing the epitope from the encoded protein. Hence the methods, provided herein permit sorting (i.e., reduction of diversity) of diverse collections. A sort that involves one step will substantially reduce diversity. The use of an optional sorting steps generally reduces diversity of less than 10, generally one.

### Dividing the Master Library

As noted above, the first step in the sorting processes herein includes dividing the master library into N sublibraries. As described above, the “D” sequence and tags can be introduced into the master library, which is then subdivided using the different D's for amplification into “N” sublibraries.

As noted above, the inclusion of “D” is optional; division can be effected by physically dividing the master library into sublibraries, and then introducing the “E” tag-encoding or “EC” tag-encoding sequences into the sublibraries. This is generally done when the initial library is very large so that the resulting sublibraries are large to ensure a uniform distribution of tags.

3. Creating the Master Library for Sorting

In this step, tags that encode each of the epitopes linked to each of the divider sequences are incorporated into the master libray, which is typically a cDNA library. Any way known to those of skill in the art to add and incorporate a double stranded DNA fragment into nucleic acid may be used. In particular, at variety of ways are contemplated herein. These include (1) using PCR amplification to incorporate them (exemplified herein); (2) ligating them directly or via linkers (see below), the ligated product, if needed, can be amplified, and other methods described herein (see below) and that can be readily devised by those of skill in the art in light of the description herein.

In the initial tagging step, when adding the E, ED or EDC set of oligonucleotides on the constituent members of the nucleic acid library, the goal is to get an even distribution of all Eₘ and all Dₙ and to have them on only one of each type of molecule. The tags must be randomly distributed among the different molecules. As long as the number of molecules is large compared to the number of tags (so that on the average only about one of each type of molecule in the collection gets each tag), the tags are evenly distributed. Hence it is preferable to have the total number of molecules in the collection in substantial excess compared to the number of tags. Such excess is at least 100-fold, more preferably 1000-fold. The exact ratios, if necessary, can be determined empirically. In practice there should be no more molecules in the reaction than the diversity. On the average each different molecule should have a different tag and only one of each different molecule should be tagged.

To practice the methods, a library of epitope-labeled molecules is prepared by randomly introducing the tags into an unlabeled library so that each tag is randomly distributed amongst the molecules. Experiments have demonstrated that the tags can be introduced randomly and equally into a cDNA library.

The master library is divided into pools, identified as D₁-Dₙ, reacted with n number of addressable collections of antibodies, each collection containing antibodies with m different epitope specificities. Each collection, such as an array, is associated with one of the pools, such as by an optical code, ioncluding a bar code a notation or a symbol or a colored code, an electronic tag or other identifier, such as color or a identifiable chemical tag, on the collection or other such identifier. The reaction is performed under conditions whereby the epitopes bind to the antibodies specific therefor, and the resulting complexes of antibodies and eptiope-tag-labeled molecules are screened using an assay that specifically identifies molecules that have a desired property. The particular collection(s) of antibodies and antibodies with a particular tag that includes molecules with the desired property are identified, thereby also identifiying the particular Dn pool and epitope tag on the molecule, thereby reducing the diversity of the collection by n×m.

4. Methods for Epitope Tag Incorporation

Any method known to one of skill in the art to link a nucleic acid molecule encoding a polypeptide to another nucleic acid or to link polypeptide to another molecule is contemplated. For exemplification, a variety of such methods are described. As noted, they are described with particular reference to antibody capture agents, and polypeptide tags that include epitopes to which the antibodies bind, but is it to be understood that the methods herein can be practiced with any capture agent and polypeptide tag therefor.

a. Ligation to Create Circular Plasmid Vector for Introduction of Tags

As noted above, in addition to use of amplication protocols for introducing the primers into the library members, the primers may be introduced by direct ligation, such as by introduction into plasmid vectors that contain the nucleic acid that encode the tags and other desired sequences. Subcloning of a cDNA into double stranded plasmid vectors is well known to those skilled in the art. One method involves digesting purified double stranded plasmid with a site-specific restriction endonuclease to create 5′ or 3′ overhangs also known as sticky ends. The double stranded cDNA is digested with the same restriction endonuclease to generate complementary sticky ends. Alternately, blunt ends in both vector DNA and cDNA are created and used for ligation. The digested cDNA and plasmid DNA is mixed with a DNA ligase in an appropriate buffer (commonly, T4 DNA ligase and buffer obtained from New England Biolabs are used) and incubated at 16° C. to allow ligation to proceed. A portion of the ligation reaction is transformed into E. coli that has been rendered competent for uptake of DNA by a variety of methods (electroporation, or heat shock of chemically competent cells are two common methods). Aliquots of the transformation mix are plated onto semi-solid media containing the antibiotic appropriate for the plasmid used. Only those bacteria receiving a circular plasmid gives rise to a colony on this selective media. Creation of a library of unique members is performed in a similar manner, however the cDNA being inserted into the vector is a mixture of different cDNA clones. These different cDNA clones are created via a wide variety of methods known to those skilled in the art.

For directional cloning of cDNA clones, which is desirable for the creation of a library used for expression of proteins from the cDNA library, two different restriction endonucleases which generate different sticky ends are used for digestion of the plasmid. The cDNA library members are created such that they contain these two restriction endonuclease recognition sites at opposite ends of the cDNA. Alternately, different restriction endonucleases that generate complementary overhangs are used (for example digestion of the plasmid with NgoMIV and the cDNA with BspEI both leave a 5′CCGG overhang and are thus compatible for ligation). Furthermore, directional insertion of the cDNA into the plasmid vector brings the cDNA under the control of regulatory sequences contained in the vector. Regulatory sequences can include promoter, transcriptional initiation and termination sites, translational initiation and termination sequences, or RNA stabilization sequences. If desired, insertion of the cDNA also places the cDNA in the same translational reading frame with sequences coding for additional protein elements including those used for the purification of the expressed protein, those used for detection of the protein with affinity reagents, those used to direct the protein to subcellular compartments, those that signal the post-translational processing of the protein.

For example, the pBAD/gIII vector (Invitrogen, Carlsbad Calif.) contains an arabinose inducible promoter (araBAD), a ribosome binding sequence, an ATG initiation codon, the signal sequence from the M13 filamentous phage gene III protein, a myc epitope tag, a polyhistidine region, the rrnB transcriptional terminator, as well as the araC and beta-lactamase open reading frames, and the CoIE1 origin of replication. Cloning sites useful for insertion of cDNA clones are designed and/or chosen such that the inserted cDNA clones are not internally digested with the enzymes used and such that the cDNA is in the same reading frame as the desired coding regions contained in the vector. It is common to use SfiI and NotI sites for insertion of single chain antibodies (scFv) into expression vectors. Therefore, to modify the pBAD/gIII vector for expression of scFvs, oligonucleotides PDK-28 (SEQ ID No. 6) and PDK-29 (SEQ ID no. 7) are hybridized and inserted into NcoI and HindIII digested pBAD/gIII DNA. The resultant vector permits insertion of scFvs (created with standard methods such as the “Mouse scFv Module” from Amersham-Pharmacia) in the same reading frame as the gene III leader sequence and the epitope tag.

For use herein, a library of expressed proteins is subdivided using a plurality of epitope tags and the antibodies that recognize them. To create the library for expressing proteins with a plurality of epitope tags, slight modifications of the subcloning techniques described above are used. A plurality of cDNA clones are inserted into a mixture of different plasmid vectors (instead of a single type of plasmid vector) such that the resulting library contains cDNA clones tagged with the different epitope tags, and each epitope tag is represented equally. Multiple plasmid vectors are created such that they differ in the epitope tag that is translated in fusion with the inserted cDNA member. For example, if there are 1000 epitope tag sequences, 1000 different vectors are constructed; if there are 250 epitope tag sequences, 250 different vectors are constructed. Those skilled in the art understand that there are a variety of methods for construction of these vectors. For illustration the myc epitope encoding region of the pBAD/gIII plasmid is removed by digestion with XbaI and SalI restriction enzymes, and the large 4.1 kb fragment is isolated. The hybridization of oligonucleotides PDK-32 (SEQ ID No. 8) and PDK-33 (SEQ ID No. 9) creates overhangs compatible with XbaI and SalI, such that the product is inserted directionally, and encodes the epitope for the HA11 antibody (see table below). Insertion of the hybridization product of PDK-34 (SEQ ID No. 10) and PDK-35 (SEQ ID No. 11) results in a vector with the FLAG M2 epitope (see table below) in frame with the inserted cDNA.

| oligo number | oligo name | Sequence 5′ to 3′ | SEQ ID |
|---|---|---|---|
| PDK-028 | SfilNotlFor | catggcggcccagccggcctaatgagcggccgca | 6 |
| PDK-029 | SfilNotlRev | agcttgcggccgctcattaggccggctgggccgc | 7 |
| PDK-032 | HAFor | ctagaatatccgtatgatgtgccggattatgcgaatagcgccg | 8 |
| PDK-033 | HARev | tcgacggcgctattcgcataatccggcacatcatacggataaa | 9 |
| PDK-034 | M2For | ctagaagattataaagatgacgacgataaaaatagcgccg | 10 |
| PDK-035 | M2Rev | tcgacggcgctatttttatcgtcgtcatctttataatcaa | 11 |

| Antibody | Epitope name | Sequence |
|---|---|---|
| 9E10 | myc | EQKLISEEDL |
| HA.11, HA.7, or 12CA5 | HA | YPYDVPDYA |
| M1, M2, M5 | FLAG | DYKDDDDK |

Each of these vectors still shares the SfiI and NotI restriction endonuclease sites to allow subcloning of cDNA clones into the vectors. Similarly, additional oligonucleotides can be designed to encode a wide variety of epitope tags that can be inserted in the same position to create a collection of different vectors.

Plasmid DNA corresponding to the vectors containing different epitope tags is prepared using methods known to those in the art (Qiagen columns, CsCI density gradient purification, etc). Purified double stranded DNA from each of the plasmids is quantified by OD260 or other methods and then is combined in equivalent amounts prior to digestion with the two restriction enzymes, and treatment with calf intestinal phosphatase (CIP, New England Biolabs). The cDNA clones of interest are also digested with the same restriction enzymes. Digested plasmid DNA and cDNA clones are separated on agarose gels to remove unwanted sticky ends and purified from agarose slices using standard methods (Qiagen gel purification kit, GeneClean kit, etc). The cDNA clones and the mixture of plasmids are reacted in 1× ligase buffer at a 3:1 molar ratio (insert to vector) with T4 DNA ligase (New England Biolabs). Typically, a ligation reaction contains about 10 ng/μl plasmid DNA and 0.5 units/μl of T4 DNA ligase in a suitable buffer, and is incubated at 16° C. for 12 to 16 hours. The reaction is diluted 8-10 fold with sterile water, and aliquots are transformed by electroporation into TOP10F′ (electrocompetant E. coli cells from Invitrogen). Liquid medium such as SOC (see, Sambrook et al. (1989) Molecular Cloning: A Laboratory Manual, 2nd Edition, Cold Spring Harbor Laboratory Press; SOC is 2% (w/v) tryptone, 0.5% (w/v) yeast extract, 8.5 mM NaCl, 2.5 mM KCl, 10 mM MgCl₂ and 20 mM glucose at pH 7) is added, and cells are allowed to recover for 1 hour at 37° C. An aliquot of the transformation mixture is plated on LB-agar plates containing 100 μg/ml ampicillin. Plates are incubated at 37° C. for 12 to 16 hours, and then individual clones are analyzed. This analysis indicates that each of the epitope tags present in the initial mixture is represented equally in the final library.

For example, a series of plasmid vectors containing the EDC sequences is created such that each vector in the series contains a single combination of EDC sequences. For example, if there are 1000 E sequences in combination with 1000 D sequences and a single C sequence, there are 10⁶ (1000×1000×1) possible combinations and therefore 10⁶ vectors are created. Each of these vectors shares restriction endonuclease sites to allow subcloning (preferably directional) of cDNA clones into the vectors. Purified plasmid DNA from all 10⁶ vectors is mixed and then digested with the restriction endonucleases. Alternatively, DNA representing each vector is digested and then mixed to create the pool of recipient vectors. Double stranded cDNA representing the library of interest is also digested with restriction endonucleases to create ends that are compatible for ligation to the ends created by vector digestion. This is accomplished by using the same enzymes for vector and cDNA digestion or by using those that generate complementary overhangs (for example NgoMIV and BspEI both leave a 5′CCGG overhang and are thus compatible for ligation). Alternately, blunt ends in both vector DNA and cDNA are created and used for ligation. Digested cDNA clones and digested vector DNAs are ligated using a DNA ligase such as T4 DNA ligase, E. coli DNA ligase, Taq DNA ligase or other comparable enzyme in an appropriate reaction buffer. The resultant DNA is transformed into bacteria, yeast, or used directly as template for in vitro transcription of RNA. The design of the vectors is such that insertion of the cDNA at the restriction endonuclease sites places the cDNA under control of promoter sequences to allow expression of the cDNA. Additionally the cDNA are in the same reading frame as the E sequence such that upon protein expression from this vector, a fusion protein containing the cDNA-encoded polypeptide fused to the epitope tag is produced. The E sequence is positioned in the vector such that the encoded epitope tag is fused to either the N or the C terminus of the resultant protein. (for restriction enzyme digestion, DNA ligation, and transformation, see, e.g., see, Sambrook et al. (1989) Molecular Cloning: A Laboratory Manual, 2nd Edition, Cold Spring Harbor Laboratory Press, Chapter 1).

b. Ligation of Sequences Resulting in Linear Tagged cDNA

Following creation of the cDNA library, sequences are appended to cDNA clones via ligation. Linear, double stranded DNA containing each of the EDC sequence combinations is created via various methods (synthesis, digestion out of plasmid containing the sequences, assembly of shorter oligonucleotides, etc.). These linear dsDNAs containing the different EDC sequences, are mixed such that each individual is equally represented in the mixture. This mixture is combined with the double stranded cDNA library and ligated using a nucleic acid ligase in an appropriate buffer. This is preferably a DNA ligase, but an RNA ligase is used if the EDC tags are composed of RNA or are RNA/DNA hybrid molecules and the library is also in the form of an RNA or RNA/DNA hybrid. In one embodiment, the EDC sequence is blunt-ended on both ends yet only one end is phosphorylated such that ligation occurs in a directional manner (with respect to the EDC sequence) and the E sequence are brought into the same reading frame as the cDNA (at either the N or C terminus of the resulting protein). In another embodiment, the EDC sequence is blunt-ended at one end and has an overhang on the other end such that ligation occurs in a directional manner (see, Sambrook et al. (1989) Molecular Cloning: A Laboratory Manual, 2nd Edition, Cold Spring Harbor Laboratory Press Chapter 8). The EDC sequences can be continuously double stranded, or partially double stranded with a single stranded central portion.

In another embodiment, the cDNA library is created to contain a restriction endonuclease site and the same restriction site is included in the EDC sequences such that upon digestion of each with the appropriate enzyme, compatible ends are created. The digested library is ligated to a mixture of digested EDC sequences using a DNA ligase in an appropriate buffer. In another embodiment, the cDNA library is created to contain a restriction endonuclease site and the EDC sequences are designed to contain a restriction site that leaves an overhang compatible to the overhang generated on the cDNA. Upon ligation of these two compatible sites, a sequence is generated that is not susceptible to cleavage with either of the enzymes used to generate the overhangs. In this case, the products of the ligation reaction are digested with the enzymes used to generate the overhangs. Alternately, the ligation reaction occurs in the presence of the enzymes used to generate the overhangs (Biotechniques August 1999; 27(2):328-30, 332-4, Biotechniques January 1992; 12(1):28, 30).

This method reduces and/or eliminates the ligation of cDNA to cDNA or EDC sequence to EDC sequence, and thus enrich for the cDNA-EDC product. Pairs of enzymes capable of generating such compatible overhangs include AgeI/XmaI, AscI/MluI, BspEI/NgoMIV, NcoI/PciI and others (New England Biolabs 2000-2001 catalog p184 and 218 for partial list). The EDC sequences and the cDNA are designed such that they are in the same reading frame following ligation. Therefore, upon protein expression from this construct, a fusion protein containing the cDNA-encoded polypeptide fused to the epitope tag is produced. The E sequence is positioned in the final construct such that the encoded epitope tag is fused to either the N or the C terminus of the resultant protein.

In another embodiment, the cDNA, the EDC sequence or both are created such that they contain a region with RNA hybridized to DNA. The RNA can be removed by digestion with the appropriate RNAse (including type 2 RNAse H) such that a single stranded DNA overhang results. This overhang can be ligated to compatible overhangs generated either by the above method or by restriction endonuclease digestion. Additionally, overhangs and flanking sequence are designed in such a way that if an EDC sequence is ligated to another EDC sequence, the resulting sequence is susceptible to digestion with a particular restriction enzyme. Likewise, if a cDNA is ligated to another cDNA, the resulting sequence is susceptible to cleavage by another restriction enzyme. Ligation reactions occur in the presence of those restriction enzymes, or are subsequently treated with those enzymes to reduce the incidence of cDNA-cDNA or EDC-EDC ligation events (see enzymes pairs and references above). The EDC sequences and the cDNA are designed such that they are in the same reading frame following ligation. Therefore, upon protein expression from this construct, a fusion protein containing the cDNA-encoded polypeptide fused to the epitope tag is produced. The E sequence is positioned in the final construct such that the encoded epitope tag is fused to either the N or the C terminus of the resultant protein. In another embodiment, PCR is used to generate the cDNA and the various EDC sequences using PCR primers that contain regions of RNA sequence that cannot be copied by certain thermostable DNA polymerases. Therefore RNA overhangs remain that can be ligated to complementary overhangs generated by the same method or by restriction enzyme digestion. RNA or DNA overhang cloning is described by Coljee et al (Nat Biotechnol July 2000; 18(7):789-91).

In another embodiment, an EDC sequence is brought into close apposition to a cDNA sequence by hybridization to a splint oligonucleotide that is complementary to the 3′ region of the cDNA and also the 5′ region of the EDC sequence (Landegen et al., Science 241:487, 1988). Joining of the cDNA and EDC is accomplished by a nucleic acid ligase under appropriate reaction conditions. In another embodiment, the splint oligonucleotide is complementary to the 5′ region of the cDNA and the 3′ region of the EDC sequence. In both cases, the different members of the CDNA library share a common sequence (at the 3′ or 5′ end), and the different EDC sequences also share a common sequence (at the 5′ or 3′ end), such that a single splint oligonucleotide sequence can hybridize to any member of the cDNA library and also to any individual of the series of EDC sequences. In each of these embodiments, the splint oligonucleotide, the cDNA and the EDC sequences can be single or double stranded DNA, or combinations of DNA and RNA. Mixtures of cDNA, EDC sequences and splint oligonucleotides are denatured at elevated temperatures to eliminate secondary structure and existing hybridization. The reaction is then cooled to allow hybridization to occur. In cases where the splint oligonucleotide is present in molar excess, a hybridization product containing the three desired components (cDNA, EDC and splint oligonucleotide) is obtained. A nucleic acid ligase is added and the reaction is incubated under appropriate conditions.

In another embodiment, the splint oligonucleotide, cDNA library and EDC sequences are designed as in the above example. The ligase chain reaction (see, e.g., LCR, F. Barany (1991) The Ligase Chain Reaction in a PCR World, PCR Methods and Applications, vol. 1 pp. 5-16; see, also, U.S. Pat. No. 5,494,810) is then performed using multiple cycles of denaturation, hybridization, and ligation with a thermostable ligase. For geometric amplification of cDNA-EDC product, double stranded cDNA and double stranded EDC sequences are needed.

C. Primer Extension and PCR for Tag Incorporation

In another embodiment, the EDC sequences are appended to the cDNA clones during the creation of the cDNA library. In this case, the EDC sequence is designed such that it can hybridize to a desired population of mRNA. This EDC serves as a primer and the RNA serves as a template for synthesis of DNA using reverse transcriptase (AMV-RT, M-MuLV-RT or other enzyme that synthesizes DNA complementary to RNA as template). The newly synthesized cDNA is complementary to the RNA and has an EDC sequence at the 5′ end. Second strand synthesis using a DNA polymerase results in double stranded DNA with the EDC at the end corresponding to the 3′ end of the RNA. In this embodiment, all members in the series of EDC sequences share a common 3′ end for hybridization to the RNA (e.g., in the case of a library of similar members of a gene family). Alternately, EDC sequences have a sequence of random nucleotides at the 3′ end for random priming of RNA (Molecular cloning: a laboratory manual 2^{nd} edition, Sambrook et al, Chapter 8).

In another embodiment, the polymerase chain reaction (PCR) is used to append EDC sequences to cDNA clones. A cDNA library is created in such a way that all members share a common sequence at the 3′ end (e.g. prime first strand cDNA synthesis with an oligonucleotide containing this common sequence, or ligation of linker sequences to double stranded cDNA clones). Additionally, each member of the cDNA library share a different common sequence (“C”) at the 5′ end. Each unique member in the series of EDC sequences have a common 3′ end that is complementary to one of the common regions in the cDNA. This mixture of EDC sequences serve as one of the amplification primers in a polymerase chain reaction. An oligonucleotide complementary to the common region at the opposite end of the cDNA serve as the second amplification primer. The cDNA library is mixed with the series of EDC amplification primers, the second primer and a thermostable polymerase (Taq, Vent, Pfu, etc) in the appropriate buffer conditions and multiple cycles of denaturation, hybridization, and DNA polymerization are executed. Alternatively, the cDNA library is subdivided after the addition of the common sequences, and aliquots are combined with individual EDC sequences, the second primer and a thermostable polymerase (Taq, Vent, Pfu, etc) in the appropriate buffer conditions and multiple cycles of denaturation, hybridization, and DNA polymerization are executed.

d. Insertion by Gene Shuffling

In another embodiment, EDC sequences are appended to cDNA clones via “DNA shuffling” or molecular breeding (see, e.g., Gene Oct. 16, 1995;164(1):49-53; Proc Natl Acad Sci U S A. Oct. 25, 1994;91(22):10747-51; U.S. Pat. No. 6,117,679). Each member in the series of EDC sequences have a common 3′ end that is complementary to one of the common regions in the cDNA library members. During creation, or mutagenesis of the cDNA library, EDC sequences are included in the PCR reaction to allow the EDC sequences to be assembled along with the fragments of the cDNA clones.

e. Recombination Strategies

Recombination strategies can also be used for introduction of tags into cDNA clones. For example, triple-helix induced recombination is used to append EDC sequences to cDNA clones. A cDNA library is created in such a way that all members share a common sequence at one end. The series of EDC sequences is designed to include a region with considerable homology to the common sequence in the cDNA library. The EDC sequences and the cDNA library are combined in a cell free recombination system (J Biol Chem May 25, 2001;276(21):18018-23) with a third homologous oligonucleotide and recombination is allowed to occur.

In another embodiment, site-specific recombination is used to append EDC sequences to cDNA clones. Site specific recombination systems include loxP/cre (U.S. Pat. No. 6,171,861; U.S. Pat. No. 6,143,557;), FLP/FRT (Broach et al. Cell 29:227-234 (1982)), the Lambda integrase with attB and attP sites (U.S. Pat. No. 5,888,732), and a multitude of others. The series of EDC sequences as well as the members of the cDNA library are designed to include a common sequence recognized by the recombinase protein (e.g. IoxP sites). The EDC sequences and the cDNA library are combined in a cell free recombination system (Protein Expr Purif June 2001;22(1):135-40) including the site specific recombinase (e.g. cre recombinase) under appropriate conditions to allow recombination to take place. Alternately, the recombination events take place inside cells such as bacteria, fungus, or higher eukaryotic cells expressing the desired recombinase (see U.S. Pat. Nos. 5,916,804, 6,174,708 and 6,140,129 as example).

In another embodiment, homologous recombination in cells is used to append EDC sequences to cDNA clones. E. coli (Nat Genet October 1998;20(2):123-8), yeast (Biotechniques March 2001;30(3):520-3), and mammalian cells (Cold Spring Harb Symp Quant Biol. 1984;49:191-7) are used for recombination of DNA segments. The EDC sequences are designed to contain both 5′ and 3′ regions with homology to two separate regions in a plasmid vector containing the cDNA. The lengths of homologous regions are dependent on the cell type being used. The cDNA and the EDC sequences are co-transformed into the cells and homologous recombination is carried out by recombination/repair enzymes expressed in the cell (see, e.g., U.S. Pat. No. 6,238,923).

f. Incorporation by Transposases

In another embodiment, transposases are used to transfer EDC sequences to cDNA clones. Integration of transposons can be random or highly specific. Transposons such as Tn7 is highly site-specific and is used to move segments of DNA (Lucklow et al., J. Virol. 67:4566-4579 (1993). The EDC sequences are contained between inverted repeat sequences (specific to the transposase used). The members of the cDNA library (or the plasmid vectors they are in) contain the target sequence recognized by the transposase (e.g attTn7). In vitro or in vivo transposition reactions insert the EDC sequences into this site.

g. Incorporation by Splicing

In another embodiment, EDC sequences flanked by RNA splice acceptor and donor sequences are inserted into the genome of various cell lines in such a way as to incorporate them into the mRNA being transcribed and translated (See U.S. Pat. No. 6,096,717 and U.S. Pat. No. 5,948,677). Proteins isolated from these organisms, or cell lines therefore contain the epitope tags and are amenable to separation by our collection of antibodies.

In another embodiment, EDC sequences are appended to library members via trans-splicing of RNA. The RNA form of EDC sequences, and preceded by RNA splice acceptor sequences, or followed by splice donor sequences are expressed in cells that then receive the library of cDNA clones. Trans-splicing of RNA (Nat Biotechnol March 1999;17(3):246-52, and U.S. Pat. No. 6,013,487) append the EDC sequence to the library member.

4. First Sorting Step

For sorting in embodiments in which the proteins are encoded by a nucleic acid library, the proteins are produced from the nucleic acids that contain the pre-selected tags. At least one up to a series of sorting steps are performed. In the first step, a first tag is introduced into the nucleic acid by direct linkage or by primer incorporation of oligonucleotides that encode the epitope Eₘ and divider regions Dₙ to create a master library. Each nucleic acid molecule includes a region at one end that encodes one of the m epitopes and one of the n dividers.

In the next step, each of n samples is amplified with a primer that comprises Dₙ to produce n sets of amplified nucleic acid samples, where each sample contains amplified sequences that contain primarily a single Dₙ and all of the E's (E₁-Eₘ). An aliquot or portion of all of each of the n samples is translated to produce n translated samples. Proteins from each of the “n” translated reactions are contacted with one of the capture agent, such as antibody, collections, where each of the capture agents in the collection specifically reacts with an Eₘ; and each of the capture agents, such as antibodies, can be identified and produces capture-agent-protein complexes via specific binding of the capture agents to the polypeptide tags.

The resulting complexes are screened, preferably using a chromogenic, luminescent or fluorgenic reporter to identify those that have bound to a protein of interest, thereby identifying the Eₘ and Dₙ that is linked to a protein of interest.

5. The Second Sorting Step

If the diversity of the proteins to be sorted is such that multiple possible proteins are identified after the initial sort, additional sorting steps may be employed. Alternatively, routine or other screening methods may be used to identify proteins of interest from the identified proteins. If the diversity at this stage is relatively low (1 to about 5000 or so, for example), the sample that contains the identified Dₙ can be screened using routine or standard screening procedures, or subjected to a second sorting step to further reduce the diversity.

Thus, if the diversity after the first sort is fairly high (such as about 100 more, or 500 or more or 10³ or more, or, depending upon the application and desired result, whatever the skilled artisan deems too high to screen by other methods), additional sorting steps are performed.

For these additional steps, the nucleic acid in the sample that contains the identified Dₙ is ampified with a set of primers that each contains a portion (designated FAₚ) of each epitope-encoding tag (each designated Eₚ) sufficient to amplify the linked nucleic acid, but insuffient to reintroduce Eₚ, where each primer includes or is of a sequence of nucleotides of formula HO-FA-Eₚ, where p is an integer of 1 to m. This amplification introduces a different one of the epitope-encoding sequences into the nucleic acid to produce a collection of cDNA clones (a sublibrary of the original) that again contains all of the epitopes distributed among the sublibrary members.

In this second sorting step, if amplification is used to introduce the new set of tags, concatamer formation can be miminized by using a low concentration of the FA primers followed by an excess of primers encoding the common region, which region is introduced by the FA primer. After the FA primer is used, the common primers out compete the FA primers for incorporation, since the C region will then be incorporated into the template nucleic acid molecule.

Alternatively, as noted above, the new set of epitope-encoding sequences can be ligated via linkers to to the template. To do this the template can be cut with a unique restriction enzyme and the linkers ligated. This can get rid of the existing epitope encoding nucleic acid and replace it with a new set of epitopes. Ligation can be followed by amplification with the common region. Other methods may also be used.

In creating the sublibrary for the second sorting step, as with the master library, it is necessary to use conditions that ensure that on the average each different molecule has a different tag and one of each kind is tagged. In this round, one tag, on the average, should attach to each of the different molecules. In this round, however, the diversity is much lower, since the first sorting step achieves an m×n reduction in diversity. Anyu of the methods described above to attach and distribute polypeptide tag-encoding sequences among the sublibrary members can be used.

Selecting the appropriate stoichiometry assures that a different tag gets on each different member in the library. The number of epitope-encoding molecules should be small relative the number of molecules in the sublibrary, thereby ensuring an even distribution thereof among the population of different molecules, such that the probability that any particular tag ends up on any particular library member is small. As with the first sorting step and preparation of the master library, preferable ratios and concentrations can be empirically determined by varying them and testing.

The nucleic acids in the resulting sublibrary are translated and the translated proteins contacted, such as under western blotting conditions, with one collection of capture agents (or a plurality of replicas thereof), such as antibodies, to form capture agent-protein complexes. The proteins in the complexes are screened to identify the capture agent, such as antibody or receptor, locus (or loci) that binds to the epitope linked to the protein of interest, thereby identifying the “E”, the eptiope sequence associated with the protein of interst. Nucleic acid molecules in the sublibrary that contain the identified “E”, epitope sequence, designated E_{q}, are specifically amplifed, with primers that include the formula 5′FBₛ 3′ (or 5′CFBₛ3′), where each FB is sufficient to amplify the linked nucleic acid using an Eₘ portion of the epitope sequence and includes all or a portion of the Eₘ. This specifically amplifies the nucleic acid molecule of interest.

In summary, the diversity (Div) equals the total number of different molecules in a library (i.e., 10⁸), N=number of divisions D₁-Dₙ, which is the number of different collections of capture agents, such as 10²; M=number of different epitope tags (and capture agents) E₁-Eₘ, such as 10³. To start the method, a master tagged library is prepared, and divided N times. Portions of the N samples are translated and spotted onto N arrays each containing M capture agents (sort 1). At this stage M×N=10⁵. For the second sort, “M” new epitopes, such as 10³ are used, the nucleic acid is translated and sorted onto one array of 10³ capture agents, sucha as antibodies, thereby achieving a 10⁸ reduction in diversity. As a result, each locus (or member of a collection if provided linked to particulate identifiable supports) in the array has a single type of protein as well as a single capturea agents. The number of sorting steps can be any desired number, but is typically one or two. If a higher number of sorts are performed, then the sensitivity of the detection assay at the first sort should be very high, since, as a result of the diversity, the concentration of the protein of interest will be low. As noted above, M and N may be different each sorting step.

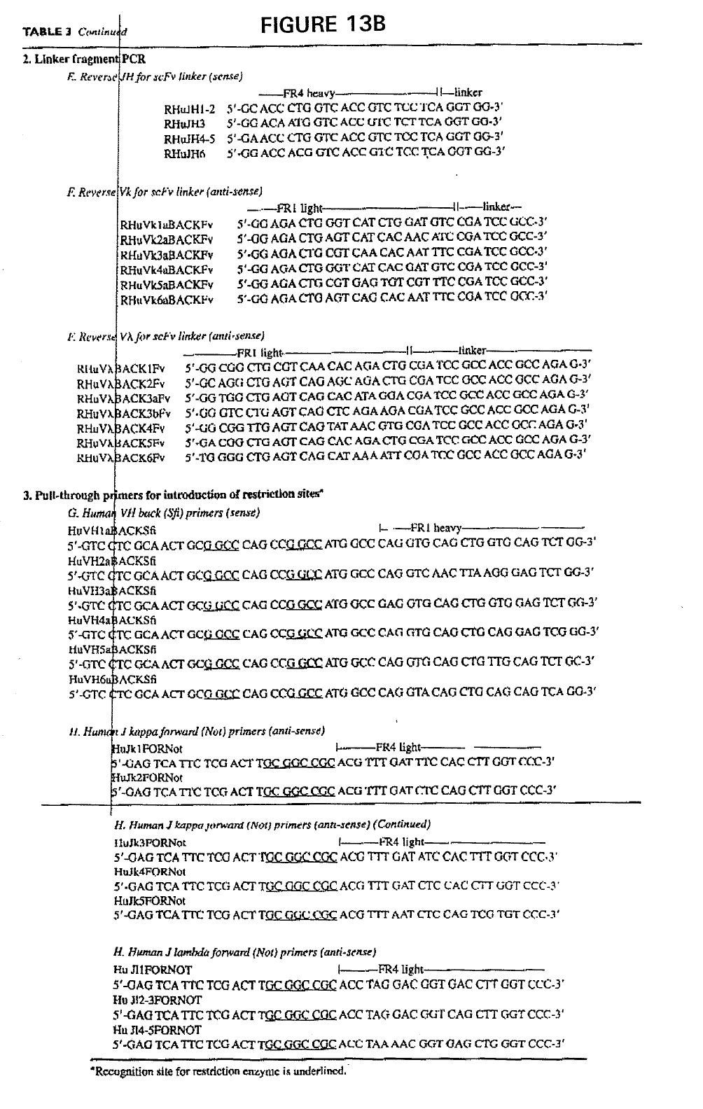

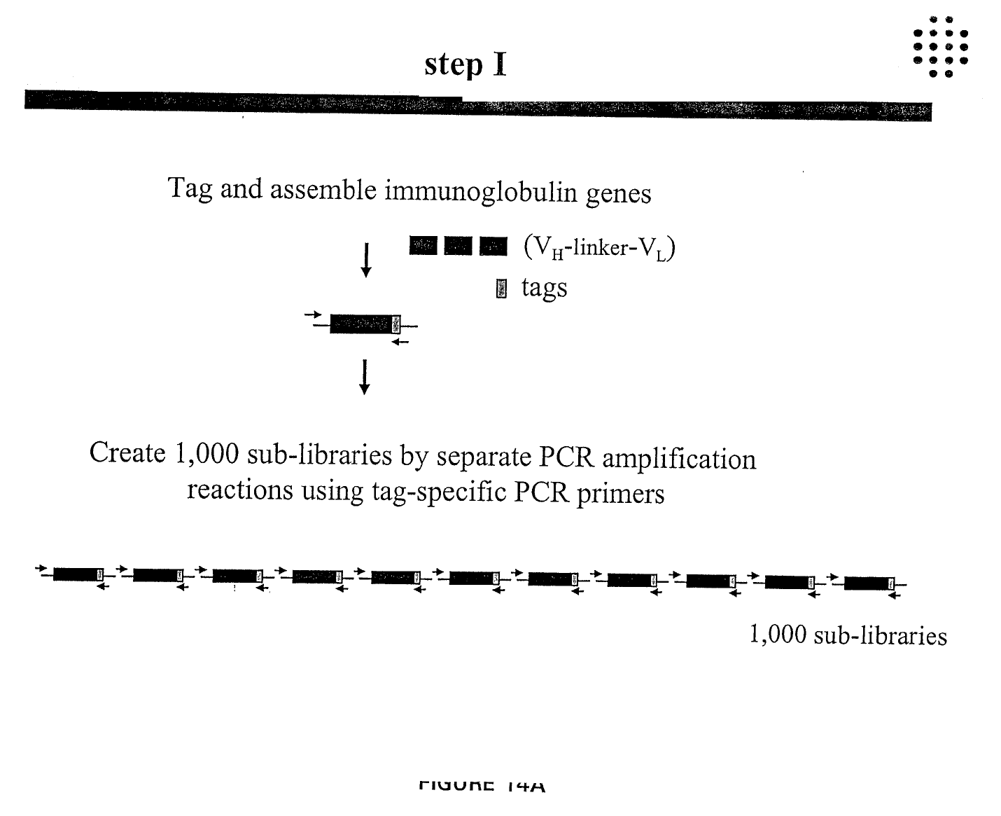

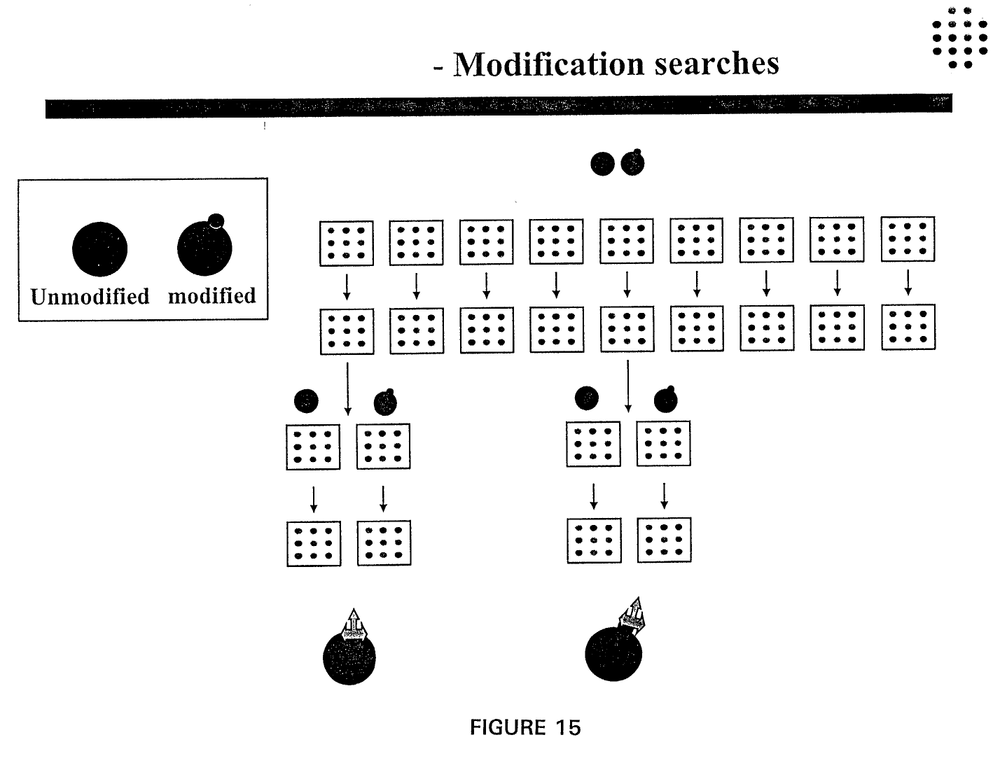

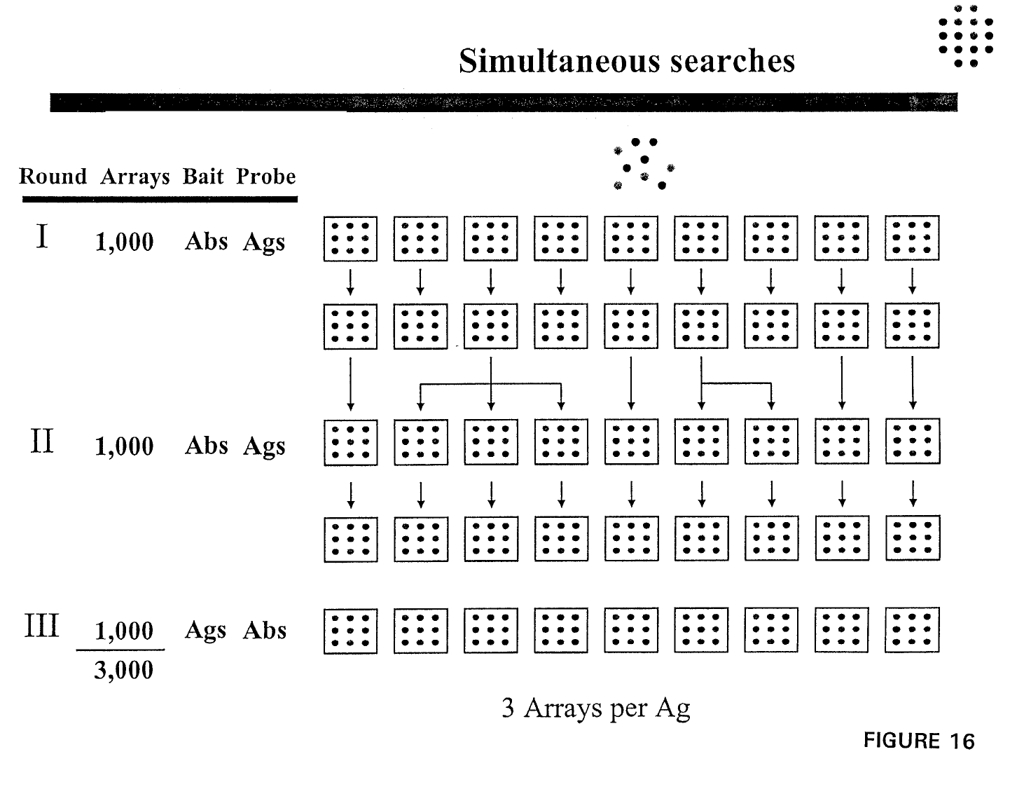

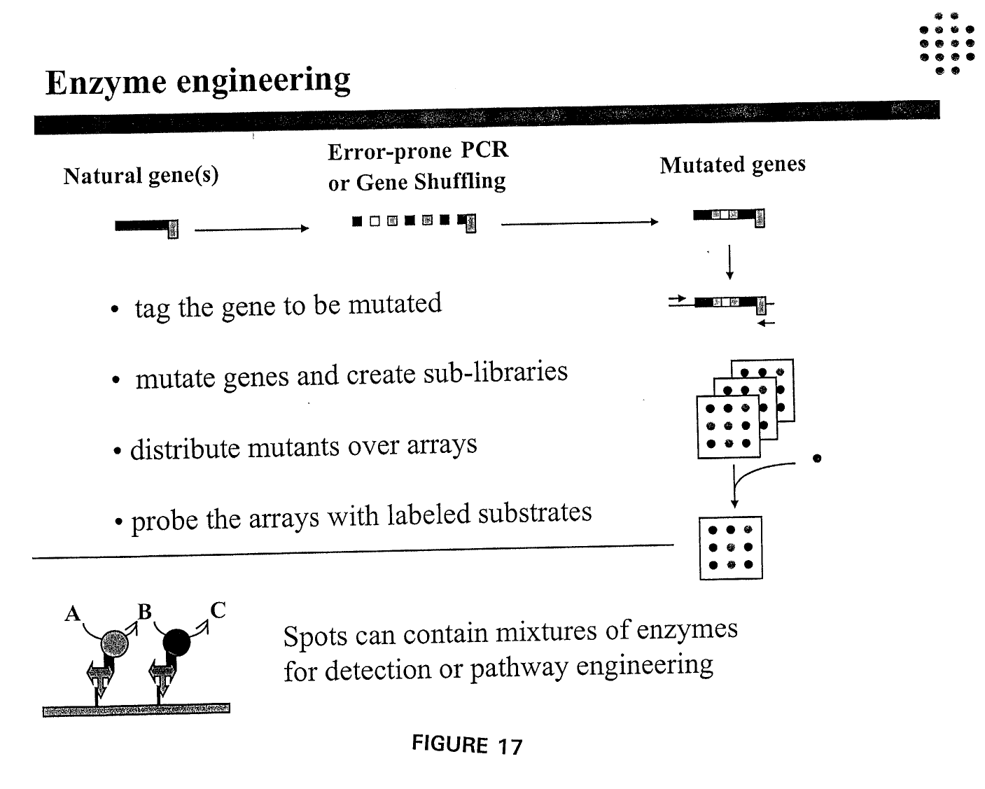

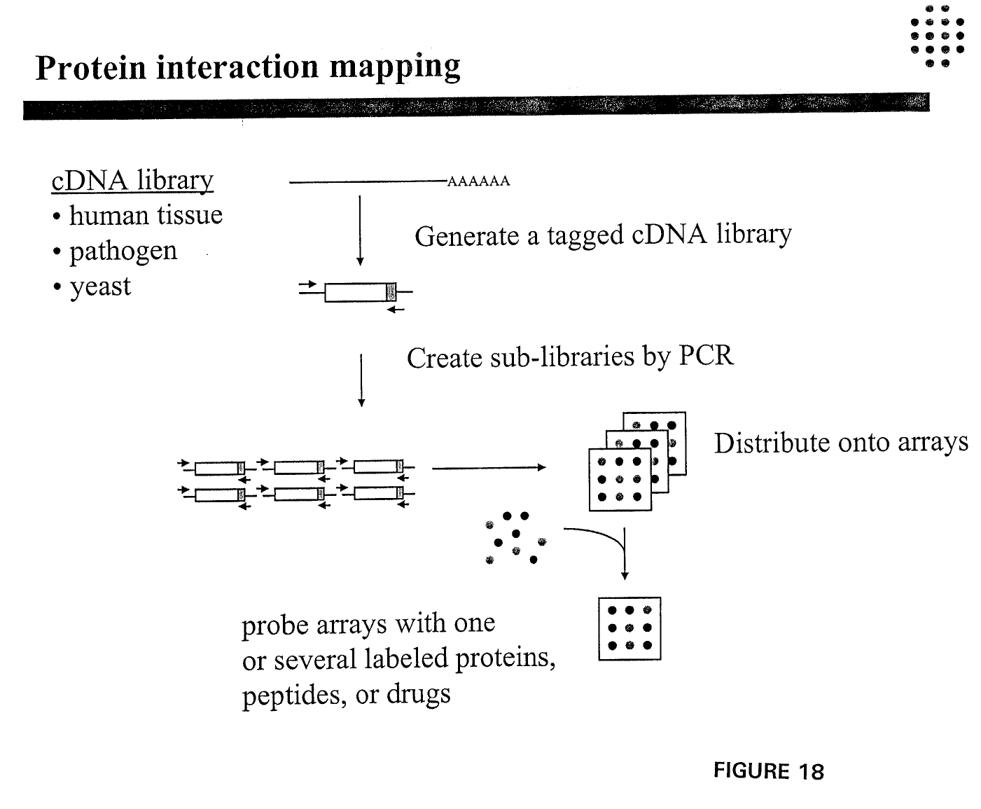

The process of nested sorting, which is applicable to sorting a variety of collections of molecules, particularly collections of proteins, DNA, small molecules and other collections is exemplified in FIGS. 1-18. The concept of nested sorting is illustrated in FIG. 1. In this example, a master collection containing 74,088 different items, such as cDNA, is searched by randomly dividing the collection into 42 sublibrarys (F1 sublibrarys). After identifying which of the 42 F1 sublibrarys contains the item of interest, such as by binding or reaction with a probe or by a protein-protein specific interaction, that group is further divided randomly into 42 new sublibrarys (F2 sublibrarys) and again the sublibrary containing the item of interest is identified. A final division of the F2 sublibrary containing the item of interest produces 42 new groups, each containing only one item. The item of interest can be uniquely identified based on its sorting lineage.

In the example shown, the item of interest was identified in the fifth F1 sublibrary, the thirty first F2 sublibrary, and the sixteenth F3 sublibrary. Of the 74,088 items in the master collection, only one has the sort lineage F1₅/F2₃₁/F3₁₆.

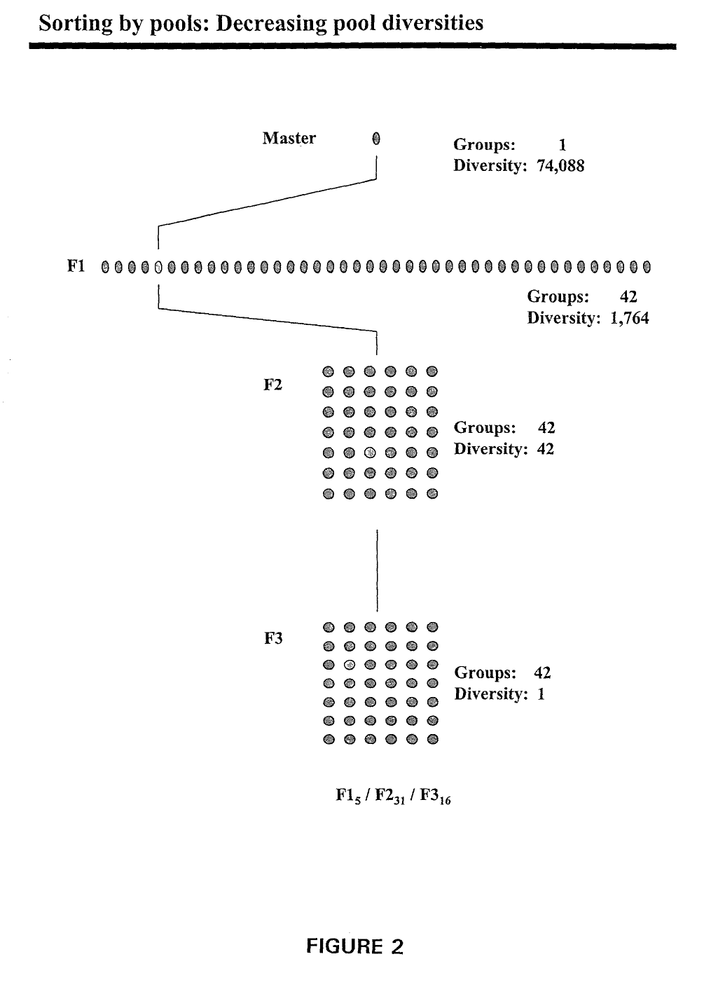

The sort illustrated in FIG. 2 is identical to the sort illustrated in FIG. 1 except that the F2 and F3 sublibraries have been arranged into arrays. This figure also illustrates that as the sort proceeds, the diversity of items within each sublibrary decreases; the exemplified master collection contains 74,088 items, the 42 F1 sublibraries contain 1,764 items each, the 42 F2 sublibraries contain 42 items, and the 42 F3 sublibraries contain only a single item. The first two figures illustrate a theoretical search based on nested sorting.

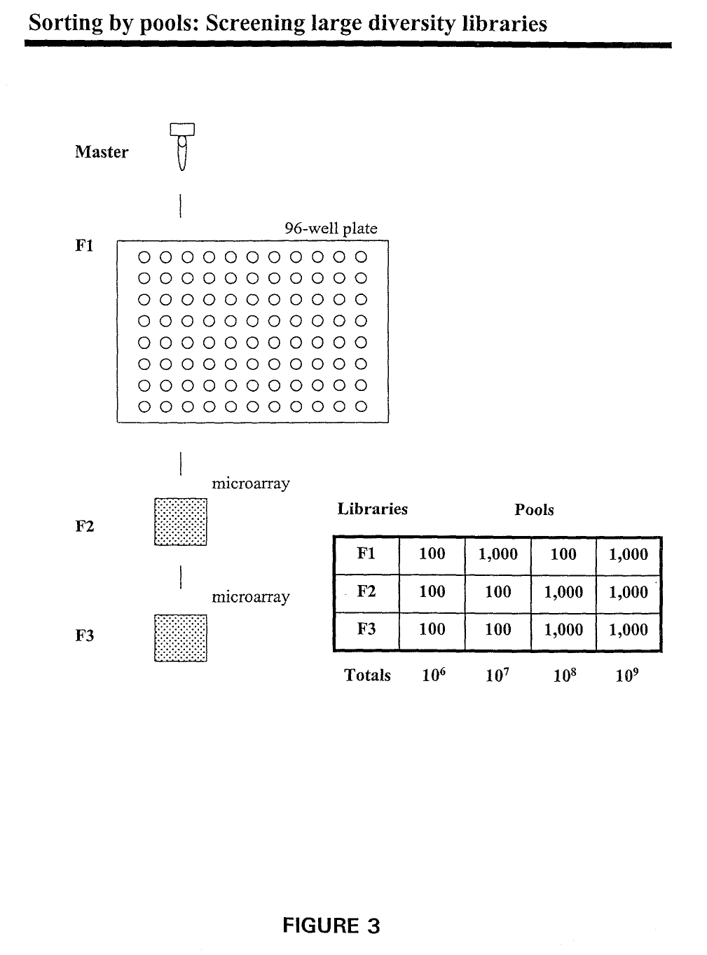

FIG. 3 illustrates the use of capture agent arrays, such as antibody arrays, as a tool for nested sorts of high diversity gene libraries. A master gene library is first randomly divided into a number of sublibrarys by separate amplification, such as PCR, reactions. The amplification reactions use sets of unique sequences of nucleotides that encode preselected epitopes and incorporate these sequences into the genes by appropriate design of primers to specifically amplify different sublibrarys of genes from the master template pool (F1 sublibrarys). These amplification reactions are performed, for example, in 96-well (or 384-well or higher density) PCR plates with a compatible thermocycler.

The amplified genes in each well are translated into their protein products and samples from each are then applied to separate capture agent collections, such as arrays (i.e., proteins from each well in the 96-well plate are applied to one of 96 capture agent arrays). The proteins by binding to capture agents, such as antibodies, in the array, sort into defined locations on the array that recognize the known unique amino acid sequences (the epitopes) that have been added to the proteins using the primers. After sorting, addresses on the array that contain the protein of interest are identified and nucleic acids from the sublibrary from which those proteins with the epitope encoding sequences that bind to the spot in the array are amplified, such as by PCR.

During this second amplification step, new sets of known epitopes are incorporated into the nucleic acid, so that they may be further sorted using additional capture agent arrays (F3).

The table in FIG. 3 illustrates how the number of initial divisions by PCR and the number of capture agents the array can be combined to search gene libraries containing, for example, from a million (10⁶) to over a billion (10⁹) different genes. For example, an initial gene library can be divided into 100 F1 sublibraries by amplification and then further divided using two arrays with capture agents recognizing 100 different epitopes. If the initial gene library contained 10⁶ different genes, the F3 addresses in the sublibraries contain a single type of gene (10⁶/100/100/100=1). An initial gene library divided into 1,000 F1 sublibraries by PCR amplification and then further divided using two arrays with capture agents recognizing 1,000 different epitopes to create the F2 and F3 sublibrarys can be used to search 10⁹ different genes (10⁹/1,000/1,000/1,000=1).

Dividing the gene libraries into sublibrarys is based on the ability of a PCR amplification reaction to specifically amplify DNA sequences using pairs of primers. Although both primers need to hybridize to sequences on either end of the template DNA, a subset of template sequences can be amplified using a primer pair in which one of the primers is common to all of the template sequences and the other primer is specific for the gene sequence of interest. For example, specific genes are often amplified from cDNA libraries using one primer that is specific for the gene of interest and another that hybridizes to the oligo(dA) tail common to all of the cDNA molecules.

6. Use of Multiple Tags in a Single Fusion Protein

The system provided herein uses epitope tags to subdivide protein libraries, such as libraries of scFvs. For example, with 1000 tags and a library of 10⁹ scFvs, there is 10⁶ scFvs for each tag. To identify a single library member, such as an scFv of interest, either a large number of individual scFvs (10⁶), are screened or more than one subdivision is employed. Using a larger number of tags a library can be reduced to small number of proteins in fewer steps.

Using a combinatorial approach, a small set of capture agent-tag pairs can be used effectively as a much larger set. By incorporating multiple tags into a protein, such as a single scFv fusion protein, better use of fewer tags can be made. For comparison, if there are 300 capture-agent tag pairs, and a library of 10⁹ members, with a single tag appended to each member, the 300 tags divide the 10⁹ members such that each type of tag is attached to 3.3×10⁶ members. With three tags incorporated into each member in a combinatorial fashion such that ⅓ of the tags are used at each of three sites, there is a total of 100×100×100 (or 10⁶) combinations. Using these 10⁶ tag combinations the 10⁹ members are divided into 1000 members per tag. Therefore in a single step with a limited number of tags, the library is effectively subdivided.

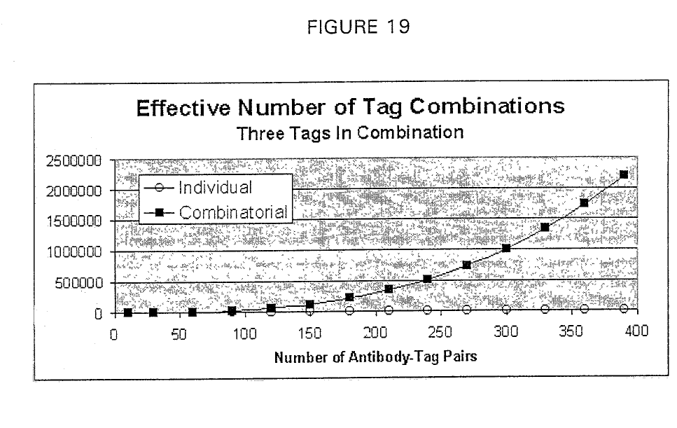

In its simplest embodiment, consider an example of x tags at site X, y tags at site Y, and z tags at site Z. If these tags are used individually, then there are x+y+z combinations. If these tags are used in combination then there are (x)(y)(z) combinations. Assuminh that the number of tags at each site (x, y and z) is one third the total (n), then for the case of individual use, C=(n/3)×3=n or there are as many total combinations (C) as there are tags; whereas for combinatorial use, there are C=(n/3)³. As the number of individual tags at each site increases, the number of combinatorial tags increases at a much higher rate (See FIG. 19). With a greater number of effective tags, the number of members of the library per tag decreases. Fewer members per tag in the initial library results in either fewer sequential rounds of screening or lower numbers of clones that to be assessed with high throughput screening.

Whether using a single tag or multiple tags in combination, the procedure is substantially the same. The protein from the expressed library is subdivided by virtue of the epitope tag binding to a capture agent, such as an antibody, against that tag. In the example presented above (using three tags in combination), each library member binds to three different anti-tag capture agents. Each combinatorial tag has its own set of addresses on an array instead of a single address. For example, if there are a total of 300 tags with 1-100 in site X, 101-200 in site Y and 201-300 in site Z, a exemplary combinatorial tag has the address X27-Y132-Z289. Other combinatorial tags also use the X27 anti-tag capture agents, such as capture agents, or the Y132 or Z289 capture agents, but no other combination uses all three. If an antigen binds to a library member tethered to the three capture agents to which each tag binds, the combinatorial tag is now known and the library member can be recovered from the original library.

Recovery of a specific library pool with a combinatorial tag is done in substantially the way a library pool with a single tag is recovered. As described herein, one way to recover subpopulations from in the library is to use the polymerase chain reaction. For exemplification, assuming that all three tags are at the C-terminus of an expressed protein such that the X tag is the most proximal to the library member, suchas an scFv, followed by the Y tag and then the Z tag. The order of DNA segments on the coding strand of cDNA is: 5′ Common>scFv>X>Y>Z 3′

A particular sub-population can be recovered by sequential rounds of PCR amplification starting with a common primer and a primer corresponding to the Z289 tag. The product from this reaction is used in the next reaction using the common primer and the Y132 tag primer. The product from this reaction is used in a subsequent reaction with the common primer and the X27 primer. After three sequential rounds of amplification, the products all correspond to libary members, such as scFvs, that were originally tagged with the X27-Y132-Z289 combination.

Those skilled in the art understand that, as long as the library has multiple nested common sequences, multiple different common primers are used in the different rounds. Those skilled in the art also understand that the multiple tags can be at opposite ends of the encoding DNA and therefore the expressed protein. It is also understood that the expressed epitope tags can be linear, constrained by disulfide bonds, constrained by a scaffold structure, expressed in loops of a fusion protein, contiguous or separated by flexible or inflexible linker sequences.

One embodiment uses, for example, a single scaffold fusion protein containing multiple sites with inserted epitope tags. This spatially separates the epitopes and allows them all to be recognized without interference with one another. The following following criteria are considered in selecting a protein scaffold: 1) known crystal structure to more easily identify surface exposed amino acids with high propensity for antigenicity, 2) free N and C-termini for fusion to the cDNA library of interest, 3) high levels of production and solubility in various protein expression systems (especially the E.coli periplasm), 4) capacity for in vitro transcription/translation, 5) absence of disulfide bonds, 6) wild-type protein is monomeric, 7) has capacity to increase solubility or function of scFvs. Using the crystal structure, positions are chosen for insertion of epitope tag libraries. These sites should be spatially separated epitopes that are relatively linear in nature (e.g. one side of an alpha helix, a turn between beta strands or a loop between helices).

D. Preparation of Antibodies

1. Antibodies and Collections of Addressable Anti-Tag Antibodies

The methods herein, rely upon the ability of the capture agents, such as antibodies, to specifically bind to the polypeptide tags, which are linked to libraries (or collections) of molecules, particularly proteins. The specificity of each antibody (or other receptor in the collection) for a particular tag is known or can be readily ascertained, such as by arraying the antibodies so that all of the antibodies at a locus in the array are specific for a particular epitope tag.

Alternatively, each antibody can be identified, such as by linkage to optically encoded tags, including colored beads or bar coded beads or supports, or linked to electronic tags, such as by providing microreactors with electronic tags or bar coded supports (see, e.g., U.S. Pat. No. 6,025,129; No. 6,017,496; No. 5,972,639; No. 5,961,923; No. 5,925,562; No. 5,874,214; No. 5,751,629; No. 5,741,462), or chemical tags (see, U.S. Pat. Nos. 5,432,018; 5,547,839) or colored tags or other such addressing methods that can be used in place of physically addressable arrays. For example, each antibody type can be bound to a support matrix associated with a color-coded tag (i.e. a colored sortable bead) or with an electronic tag, such as an radio-frequency tag (RF), such as IRORI MICROKANS® and MICROTUBES® microreactors (see, U.S. Pat. No. 6,025,129; No. 6,017,496; No. 5,972,639; No. 5,961,923; No. 5,925,562; No. 5,874,214; No. 5,751,629; No. 5,741,462; International PCT application No. WO98/31732; International PCT application No. WO98/15825; and, see, also U.S. Pat. No. 6,087,186). For the methods and collections provided herein, the antibodies of each type can be bound to the MICROKAN or MICROTUBE microreactor support matrix and the associate RF tag, bar code, color, colored bead or other identifier to serves to identify the receptors, such as antibodies, and hence the epitope tag to which the receptor, such as an antibody, binds.

For exemplary purposes herein, reference is made to antibodies and tags that encode epitopes to which the antibody specifically binds. It is understood that any pair of molecules that specifically bind are contemplated; for purposes herein the molecules, such as antibodies, are designated receptors, and the molecules, such as ligands, that bind thereto are epitopes. The epitopes are typically short sequences of amino acids that specifically bind to the receptor, such as an antibody or specific binding fragment thereof.

Also, for exemplary purposes herein, reference is made to positional arrays. It is understood, however, that such other identifying methods can be readily adapted for use with the methods herein. It is only necessary that the identity (i.e., epitope-tag specificity) of the receptor, such as an antibody, is known. The resulting collections of addressable receptors (i.e., antibodies), whether in a two-dimensional or three-dimensional array, or linked to opticially encoded beads or colored supports or RF tags or other format, can be employed in the methods herein.

By reacting a collection of antibodies with libraries of polypeptide tag-labeled molecules, and then performing screening assays to identify the members of the collection of the antibodies to which epitope-labeled molecules of a desired property have bound, a reduction in the diversity of the library of molecules is achieved. Each collection of antibodies serves as a sorting device for effecting this reduction in diversity. Repeating the process a plurality of times can effect a rapid and substantial reduction in diversity.

2. Preparation of the Capture Agents

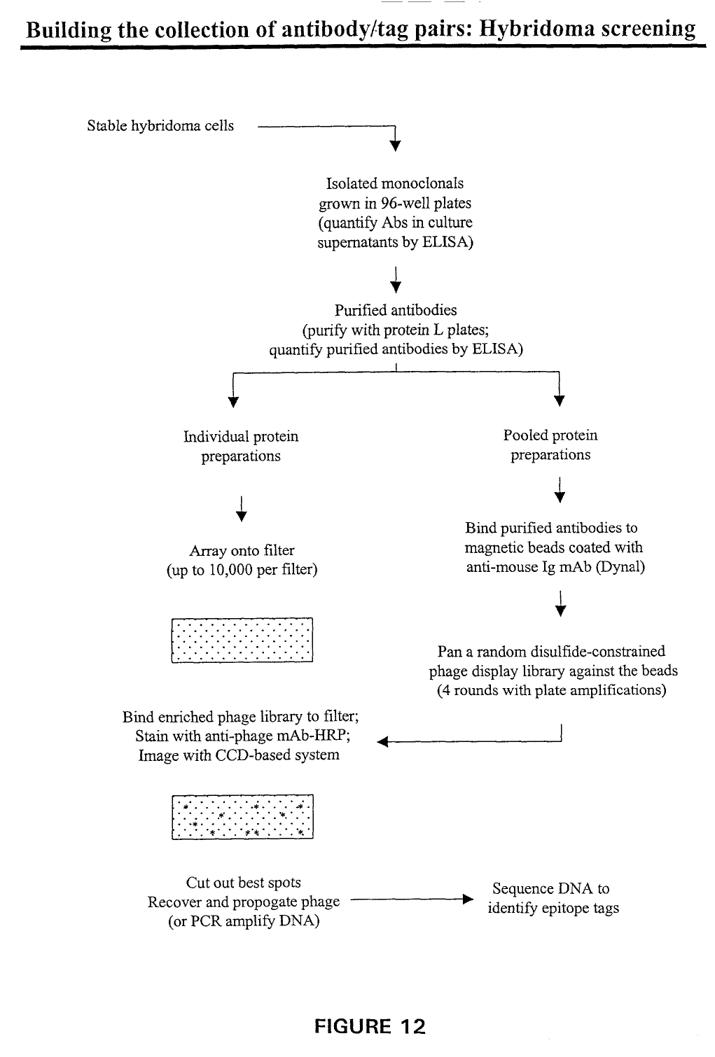

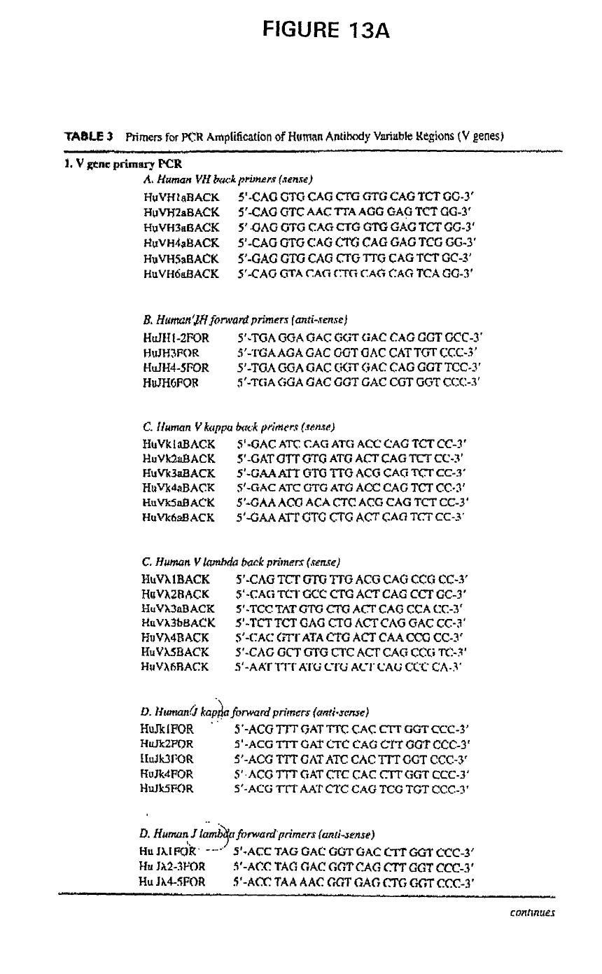

The quality of the sorts is dependent on the quality of the collection of capture agents, such as antibodies, that make up the sorting array. In addition to requirements on binding affinity and specificity, the epitopes bound by the capture agents (antibodies) in the array determine the E, FA and FB sequences used as priming sites for the the amplification reactions (PCRs). FIG. 12 outlines a high throughput screen for discovering immunoglobulin (Ig) produced from hybridoma cells for use in generating antibodies for use in the collections.

Hybridoma cells are created either from non-immunized mice or mice immunized with a protein expressing a library of random disulfide-constrained heptmeric epitopes or other random peptide libraries. Stable hybridoma cells are initially screened for high Ig production and epitope binding. Ig production is measured in culture supernatants by ELISA assay using a goat anti-mouse IgG antibody. Epitope binding is also measured by ELISA assay in which the mixture of haptens (epitope tagged proteins) used for immunization are immobilized to the ELISA plate and bound IgG from the culture supernatants is measured using a goat anti-mouse IgG antibody. Both assays are done in 96-well formats or other suitable formats. For example, approximately 10,000 hybridomas are selected from these screens.

Next, the Ig are separately purified using 96-well or higher density purification plates containing filters with immobilized Ig-binding proteins (proteins A, G or L). The quantity of purified Ig is measured using a standard protein assay formatted for 96-well or higher density plates. Low microgram quantities of Ig from each culture are expected using this purification method.

The purified Ig are spotted separately onto a nitrocellulose filter using a standard pin-style arraying system. The purified Ig are also combined to produce a mixture with equal quantities of each Ig. The mixed Ig are bound to paramagnetic beads which are used as a solid-phase support to pan a library of bacteriophage expressing the raindom disulfide-constrained heptmeric epitopes. The batch panning enriches the phage display library for phage expressing epitopes to the purified Ig. This enrichment dramatically reduces the diversity in the phage library.

The enriched phage display library is then bound to the array of purified Ig and stringently washed. Ig-binding phage are detected by staining with an anti-phage antibody-HRP conjugate to produce a chemilumminescent signal detectable with a charge coupled device (CCD)-based imaging system. Spots in the array producing the strongest signals are cut out and the phage eluted and propagated. Epitopes expressed by the recovered phage are identified by DNA sequencing and further evaluated for affinity and specificity. This method generates a collection of high-affinity, high-specificity antibodies that recognize the cognate epitopes. Continued screening produces larger collections of antibodies of improved quality.

3. Preparation of Anti-Tag Capture Agent Arrays

Each spot contains a multiplicity of capture agents, such as antibodies with a single specificity. Each spot is of a size suitable for detection. Spots on the order of 1 to 300 microns, typically 1 to 100, 1 to 50, and 1 to 10 microns, depending upon the size of the array, target molecules and otherr parameters. Generally the spots are 50 to 300 microns. In preparing the arrays, a sufficient amount is delivered to the surface to functionally cover it for dectection of proteins having the desired properties. Generally the volume of antibody-containing mixture delivered for preparation of the arrays is a nanoliter volume (1 up to about 99 nanoliters) and is generally about a nanoliter or less, typically between about 50 and about 200 picoliters. This is very roughly about 10 million to 100,000 molecules per spot, where each spot has capture agents, such as antibodies, that recognize a single epitope. For example, if there are 10 million molecules and 1000 different ones in the protein mixture reacting with the locus, there are 10⁴ of each type of molecule per spot. The size of the array and each spot should be such that positive reactions in the screening step can be imaged, preferably by imaging the entire array or a pluraity therof, such as 24, 96, or more arrays, at the same time.

A support (see below for exemplary supports), such as KODAK paper plus gelatin or other suitable matrix can be used, and then ink jet and stamping technology or other suitable dispensing methods and appartus, are used to reproducibly print the arrays. The arrays are printed with, for example, a piezo or inkjet printer or other such nanoliter or smaller volume dispensing device. For example, arrays with 1000 spots can be printed. A plurality of replicate arrays, such as 24 or 48, 96 or more can be placed on a sheet the size of a conventional 96 well plate.

Among the embodiments contemplated herein, are sheets of arrays each with replicates of the antibody array. These are prepared using, for example, a piezo or inkjet dispensing system. A large number, for example, 1000 can be printed at a time using, for example a print head with 1000 different holes (like a stamp with 500 μM holes). It can be fabricated from, for example, molded plastic with many holes, such as 1000 holes each filled with 1000 different capture agents, such as antibodies. Each hole can be linked to reservoirs that are linked to conduits of decreasing size, which ultimately dispense the capture agents, such as antibodies into the print head. Each array on the sheet can be spacially separated, and/or separated by a physical barrier, such as a plastic ridge, or a chemical barrier, such a hydrophobic barrier (i.e., hydrogels separated by hydrophobic barriers). The sheets with the arrays can be conveniently the size of a 96 well plate or higher density. Each array contains a pluralty of addressable anti-tag antibodies specific for the pre-selected set of epitope tags. For example, 33×33 arrays contain roughly 1000 antibodies, each spot on each array containing antbodies that specifically bind to a single pre-selected epitope. A plurality of arrays separated by barriers can be employed.

For dispensing the antibodies onto the surface, the goal is functional surface coverage, such that a screened desired protein is detectable. To achieve this, for example, about 1 to 2 mgs/ml from the starting collection are used and about 500 picoliters per antibody are deposited per spot on the array. The exact amount(s) can be empirically determined and depend upon several variables, such as the surface and the senstivity of the detection methods. The antibodies are preferably covalently linked, such as by sulfhydryl linkages to amides on the surface.

Other exemplary dispensing and immobilizing systems include, but are not limited to, for example, systems available from Genometrix, which has a system for printing on glass; from Illumina, which employs the tips of fiber optic cables as supports; from Texas Instruments, which has chip surface plasmon resonance (i.e., protein derivatized gold); injet systems, such as those from Microfab Technologies, Piano Tex.; Incyte, Palo Alto, Calif., Protogene, Mountain View, Calif., Packard BioSciences, Meriden Conn., and other such systems for dispensing and immobilizing proteins to suitable support surfaces. Other systems such as blunt and quill pins, solenoid and piezo nanoliter dispensers and others are also contemplated.

4. Preparation of Other Collections

The capture agents are linked to beads or other particulate supports that are identifiable. For example, the capture agents are linked to optically encoded microspheres, such as those available from Luminex, Austin Tex., the contain fluorescent dyes encapsulated therein. The microsphere, which encapsulate dyes, are prepared from any suitable material (see, e.g., International PCT application Nos. WO 01/13119 and WO 99/19515; see description below), including stryene-ethylene-butylene-styrene block copolymers, homopolymers, gelatin, polystyrene, polycarbonate, polyethylene, polypopylene, resins, glass, and any other suitable support (matrix material), and are of a size of a about a nanometer to about 10 millimeters in diameter. By virtue of the combination of, for example two different dyes at ten different concentrations, a plurality microspheres (100 in this instance), each identifiable by a unique fluoresence, are produced.

Alternatively, combinations of chromophores or colored dyes or other colored substatnces are encapsulated to produce a variety of different colors encapsulated in microspheres or other particles, which are then used as supports for the capture agents, such as antibodies. Each capture agent, such as an antibody, is linked to a particular colored bead, and, is thereby identifiable. After producing the beads with linked capture agents, such as antibodies, reaction with the epitope-tagged molecules can be performed in liquid phase. The beads that react with the epitopes are identified, and as a result of the color of the bead the particular epitope and is then known. The sublibrary from which the linked molecule is derived is then identified.

E. Supports for Immobilizing Antibodies

Supports for immobilizing the antibodies are any of the insoluble materials known for immobilization of ligands and other molecules, used in many chemical syntheses and separations, such as in affinity chromatography, in the immobilization of biologically active materials, and during chemical syntheses of biomolecules, including proteins, amino acids and other organic molecules and polymers. Suitable supports include any material, including biocompatible polymers, that can act as a support matrix for attachment of the antibody material. The support material is selected so that it does not interfere with the chemistry or biological screening reaction.

Supports that are also contemplated for use herein include fluophore-containing or -impregnated supports, such as microplates and beads (commercially available, for example, from Amersham, Arlington Heights, Ill.; plastic scintillation beads from Nuclear Technology, Inc., San Carlos, Calif. and Packard, Meriden, Conn., and colored bead-based supports (fluorescent particles encapsulated in microspheres) from Luminex Corporation, Austin, Tex. (see, International PCT application No. WO/0114589, which is based on U.S. application Ser. No. 09/147,710; see International PCT application No. WO/Ol 13119, which is U.S. application Ser. No. 09/022,537). The microspheres from Luminex, for example, are internally color-coded by virtue of the encapsulation of fluorescent particles and can be provided as a liquid array. The capture agents, such as antibodies (epitopes) are linked directly or indirectly by any suitable method and linkage or interaction to the surface of the bead and bound proteins can be identified by virtue of the color of the bead to which they are linked. Detection can be effected by any means, and can be combined with chromogenic or fluorescent detectors or reporters that result in a detectable change in the color of the microsphere (bead) by virtue of the colored reaction and color of the bead. For the bead-based arrays, the anti-tag capture agents are attached to the color-coded beads in separate reactions. The code of the bead identifies the capture agent, such as antibody, attached to it. The beads can then be mixed and subseuequent binding steps performed in solution. They can then be arrayed, for example, by packing them into a microfabricated flow chamber, with a transparent lid, that permits only a single layer of beads to form resulting in a two-dimensional array. The beads on which a protein is bound identified, thereby identifying the capture agent and the tag. The beads are imaged, for example, with a CCD camera to identify beads that have reacted. The codes of the such beads are identified, thereby identifying the captuer agent, which in turn identifies the polypeptide tag and, ultimately, the protein of interest.

The support may also be a relatively inert polymer, which can be grafted by ionizing radiation to permit attachment of a coating of polystyrene or other such polymer that can be derivatized and used as a support. Radiation grafting of monomers allows a diversity of surface characteristics to be generated on supports (see, e.g., Maeji et al. (1994) Reactive Polymers 22:203-212; and Berg et al. (1989) J. Am. Chem. Soc. 111:8024-8026). For example, radiolytic grafting of monomers, such as vinyl momomers, or mixtures of monomers, to polymers, such as polyethylene and polypropylene, produce composites that have a wide variety of surface characteristics. These methods have been used to graft polymers to insoluble supports for synthesis of peptides and other molecules

The supports are typically insoluble substrates that are solid, porous, deformable, or hard, and have any required structure and geometry, including, but not limited to: beads, pellets, disks, capillaries, hollow fibers, needles, solid fibers, random shapes, thin films and membranes, and most preferably, form solid surfaces with addressable loci. The supports may also include an inert strip, such as a teflon strip or other material to which the capture agents antibodies and other molecules do not adhere, to aid in handling the supports, and may include an identifying symbology.

The preparation of and use of such supports are well known to those of skill in this art; there are many such materials and preparations thereof known. For example, naturally-occurring materials, such as agarose and cellulose, may be isolated from their respective sources, and processed according to known protocols, and synthetic materials may be prepared in accord with known protocols. These materials include, but are not limited to, inorganics, natural polymers, and synthetic polymers, including, but are not limited to: cellulose, cellulose derivatives, acrylic resins, glass, silica gels, polystyrene, gelatin, polyvinyl pyrroliclone, co-polymers of vinyl and acrylamide, polystyrene cross-linked with divinylbenzene or the like (see, Merrifield (1964) Biochemistry 3:1385-1390), polyacrylamides, latex gels, polystyrene, dextran, polyacrylamides, rubber, silicon, plastics, nitrocellulose, celluloses, natural sponges, and many others. Selection of the supports is governed, at least in part, by their physical and chemical properties, such as solubility, functional groups, mechanical stability, surface area swelling propensity, hydrophobic or hydrophilic properties and intended use.

1. Natural Support Materials

Naturally-occurring supports include, but are not limited to agarose, other polysaccharides, collagen, celluloses and derivatives thereof, glass, silica, and alumina. Methods for isolation, modification and treatment to render them suitable for use as supports is well known to those of skill in this art (see, e.g., Hermanson et al. (1992) Immobilized Affinity Ligand Techniques, Academic Press, Inc., San Diego). Gels, such as agarose, can be readily adapted for use herein. Natural polymers such as polypeptides, proteins and carbohydrates; metalloids, such as silicon and germanium, that have semiconductive properties, may also be adapted for use herein. Also, metals such as platinum, gold, nickel, copper, zinc, tin, palladium, silver may be adapted for use herein. Other supports of interest include oxides of the metal and metalloids such as Pt—PtO, Si—SiO, Au—AuO, TiO2, Cu—CuO, and the like. Also compound semiconductors, such as lithium niobate, gallium arsenide and indium-phosphide, and nickel-coated mica surfaces, as used in preparation of molecules for observation in an atomic force microscope (see, e.g., III et al. (1993) Biophys J. 64:919) may be used as supports. Methods for preparation of such matrix materials are well known.

For example, U.S. Pat. No. 4,175,183 describes a water insoluble hydroxyalkylated cross-linked regenerated cellulose and a method for its preparation. A method of preparing the product using near stoichiometric proportions of reagents is described. Use of the product directly in gel chromatography and as an intermediate in the preparation of ion exchangers is also described.

2. Synthetic Supports

There are innumerable synthetic supports and methods for their preparation known to those of skill in this art. Synthetic supports typically produced by polymerization of functional matrices, or copolymerization from two or more monomers from a synthetic monomer and naturally occurring matrix monomer or polymer, such as agarose.

Synthetic matrices include, but are not limited to: acrylamides, dextran-derivatives and dextran co-polymers, agarose-polyacrylamide blends, other polymers and co-polymers with various functional groups, methacrylate derivatives and co-polymers, polystyrene and polystyrene copolymers (see, e.g., Merrifield (1964) Biochemistry 3:1385-1390; Berg et al. (1990) in Innovation Perspect. Solid Phase Synth. Collect. Pap., Int. Symp., 1st, Epton, Roger (Ed), pp. 453-459; Berg et al. (1989) in Pept., Proc. Eur. Pept. Symp., 20th, Jung, G. et al. (Eds), pp. 196-198; Berg et al. (1989) J. Am. Chem. Soc. 111:8024-8026; Kent et al. (1979) Isr. J. Chem. 17:243-247; Kent et al. (1978) J. Org. Chem. 43:2845-2852; Mitchell et al. (1976) Tetrahedron Lett. 42:3795-3798; U.S. Pat. Nos. 4,507,230; 4,006,117; and 5,389,449). Methods for preparation of such support matrices are well-known to those of skill in this art.

Synthetic support matrices include those made from polymers and co-polymers such as polyvinylalcohols, acrylates and acrylic acids such as polyethylene-co-acrylic acid, polyethylene-co-methacrylic acid, polyethy-lene-co-ethylacrylate, polyethylene-co-methyl acrylate, polypropylene-co-acrylic acid, polypropylene-co-methyl-acrylic acid, polypropylene-co-ethyl-acrylate, polypropylene-co-methyl acrylate, polyethylene-co-vinyl acetate, polypropylene-co-vinyl acetate, and those containing acid anhydride groups such as polyethylene-co-maleic anhydride, polypropylene-co-maleic anhydride and the like. Liposomes have also been used as solid supports for affinity purifications (Powell et al. (1989) Biotechnol. Bioeng. 33:173).

For example, U.S. Pat. No. 5,403,750, describes the preparation of polyurethane-based polymers. U.S. Pat. No. 4,241,537 describes a plant growth medium containing a hydrophilic polyurethane gel composition prepared from chain-extended polyols; random copolymerization can be peformed with up to 50% propylene oxide units so that the prepolymer is a liquid at room temperature. U.S. Pat. No. 3,939,123 describes lightly crosslinked polyurethane polymers of isocyanate terminated prepolymers containing poly(ethyleneoxy) glycols with up to 35% of a poly(propyleneoxy) glycol or a poly(butyleneoxy) glycol. In producing these polymers, an organic polyamine is used as a crosslinking agent. Other supports and preparation thereof are described in U.S. Pat. Nos. 4,177,038, 4,175,183, 4,439,585, 4,485,227, 4,569,981, 5,092,992, 5,334,640, 5,328,603.

U.S. Pat. No. 4,162,355 describes a polymer suitable for use in affinity chromatography, which is a polymer of an aminimide and a vinyl compound having at least one pendant halo-methyl group. An amine ligand, which affords sites for binding in affinity chromatography is coupled to the polymer by reaction with a portion of the pendant halo-methyl groups and the remainder of the pendant halo-methyl groups are reacted with an amine containing a pendant hydrophilic group. A method of coating a substrate with this polymer is also described. An exemplary aminimide is 1,1-dimethyl-1-(2-hydroxyoctyl)amine niethacrylimide and vinyl compound is a chloromethyl styrene.

U.S. Pat. No. 4,171,412 describes specific supoports based on hydrophilic polymeric gels, preferably of a macroporous character, which carry covalently bonded D-amino acids or peptides that contain D-amino acid units. The basic support is prepared by copolymerization of hydroxyalkyl esters or hydroxyalkylamides of acrylic and methacrylic acid with crosslinking acrylate or methacrylate comonomers are modified by the reaction with diamines, aminoacids or dicarboxylic acids and the resulting carboxyterminal or aminoterminal groups are condensed with D-analogs of aminoacids or peptides. The peptide containing D-amino-acids also can be synthesized stepwise on the surface of the carrier.

U.S. Pat. No. 4,178,439 describes a cationic ion exchanger and a method for preparation thereof. U.S. Pat. No. 4,180,524 describes chemical syntheses on a silica support.

Immobilized Artificial Membranes (IAMs; see, e.g., U.S. Pat. Nos. 4,931,498 and 4,927,879) may also be used. IAMs mimic cell membrane environments and may be used to bind molecules that preferentially associate with cell membranes (see, e.g., Pidgeon et al. (1990) Enzyme Microb. Technol. 12:149).

Among the supports contemplated herein are those described in International PCT application Nos WO 00/04389, WO 00/04382 and WO 00/04390; KODAK film supports coated with a matrix material; see also, U.S. Pat. Nos., 5,744,305 and 5,556,752 for other supports of interest. Also of interest are colored “beads”, such as those from Luminex (Austin, Tex.).

3. Immobilization and Activation

Numerous methods have been developed for the immobilization of proteins and other biomolecules onto solid or liquid supports (see, e.g., Mosbach (1976) Methods in Enzymology 44; Weetall (1975) Immobilized Enzymes, Antigens, Antibodies, and Peptides; and Kennedy et al. (1983) Solid Phase Biochemistry, Analytical and Synthetic Aspects, Scouten, ed., pp. 253-391; see, generally, Affinity Techniques. Enzyme Purification: Part B. Methods in Enzymology, Vol. 34, ed. W. B. Jakoby, M Wilchek, Acad. Press, N.Y. (1974); Immobilized Biochemicals and Affinity Chromatography, Advances in Experimental Medicine and Biology, vol. 42, ed. R. Dunlap, Plenum Press, N.Y. (1974)).

Among the most commonly used methods are absorption and adsorption or covalent binding to the support, either directly or via a linker, such as the numerous disulfide linkages, thioether bonds, hindered disulfide bonds, and covalent bonds between free reactive groups, such as amine and thiol groups, known to those of skill in art (see, e.g., the PIERCE CATALOG, ImmunoTechnology Catalog & Handbook, 1992-1993, which describes the preparation of and use of such reagents and provides a commercial source for such reagents; and Wong (1993) Chemistry of Protein Conjugation and Cross Linking, CRC Press; see, also DeWitt et al. (1993) Proc. Natl. Acad. Sci. U.S.A. 90:6909; Zuckermann etal. (1992) J. Am. Chem. Soc. 114:10646; Kurth et a. (1994) J. Am. Chem. Soc. 116:2661; Ellman et al. (1994) Proc. Natl. Acad. Sci. U.S.A. 91:4708; Sucholeiki (1994) Tetrahedron Lttrs. 35:7307; and Su-Sun Wang (1976) J. Org. Chem. 41:3258; Padwa et al. (1971) J. Org. Chem. 41:3550 and Vedejs et al. (1984) J. Org. Chem. 49:575, which describe photo-sensitive linkers).

To effect immobilization, a solution of the protein or other biomolecule is contacted with a support material such as alumina, carbon, an ion-exchange resin, cellulose, glass or a ceramic. Fluorocarbon polymers have been used as supports to which biomolecules have been attached by adsorption (see, U.S. Pat. No. 3,843,443; Published International PCT Application WO/86 03840) A large variety of methods are known for attaching biological molecules, including proteins and nucleic acids, molecules to solid supports (see. e.g., U.S. Pat. No. 5451683). For example, U.S. Pat. No. 4,681,870 describes a method for introducing free amino or carboxyl groups onto a silica support. These groups may subsequently be covalently linked to other groups, such as a protein or other anti-ligand, in the presence of a carbodiimide. Alternatively, a silica matrix may be activated by treatment with a cyanogen halide under alkaline conditions. The anti-ligand is covalently attached to the surface upon addition to the activated surface. Another method involves modification of a polymer surface through the successive application of multiple layers of biotin, avidin and extenders (see, e.g., U.S. Pat. No. 4,282,287); other methods involve photoactivation in which a polypeptide chain is attached to a solid substrate by incorporating a light-sensitive unnatural amino acid group into the polypeptide chain and exposing the product to low-energy ultraviolet light (see, e.g., U.S. Pat. No. 4,762,881). Oligonucleotides have also been attached using photochemically active reagents, such as a psoralen compound, and a coupling agent, which attaches the photoreagent to the substrate (see, e.g., U.S. Pat. No. 4,542,1C2 and U.S. Pat. No. 4,562,157). Photoactivation of the photoreagent binds a nucleic acid molecule to the substrate to give a surface-bound probe.

Covalent binding of the protein or other biomolecule or organic molecule or biological particle to chemically activated solid matrix supports such as glass, synthetic polymers, and cross-linked polysaccharides is a more frequently used immobilization technique. The molecule or biological particle may be directly linked to the matrix support or linked via a linker, such as a metal (see, e.g., U.S. Pat. No. 4,179,402; and Smith et al. (1992) Methods: A Companion to Methods in Enz. 4:73-78). An example of this method is the cyanogen bromide activation of polysaccharide supports, such as agarose. The use of perfluorocarbon polymer-based supports for enzyme immobilization and affinity chromatography is described in U.S. Pat. No. 4,885,250). In this method the biomolecule is first modified by reaction with a perfluoroalkylating agent such as perfluorooctylpropylisocyanate described in U.S. Pat. No. 4,954,444. Then, the modified protein is adsorbed onto the fluorocarbon support to effect immobilization.

The activation and use of supports are well known and may be effected by any such known methods (see, e.g., Hermanson et al. (1992) Immobilized Affinity Ligand Techniques, Academic Press, Inc., San Diego). For example, the coupling of the amino acids may be accomplished by techniques familiar to those in the art and provided, for example, in Stewart and Young, 1984, Solid Phase Synthesis, Second Edition, Pierce Chemical Co., Rockford.

Molecules may also be attached to supports through kinetically inert metal ion linkages, such as Co(III), using, for example, native metal binding sites on the molecules, such as IgG binding sequences, or genetically modified proteins that bind metal ions (see, e.g., Smith et al. (1992) Methods: A Companion to Methods in Enzymology 4, 73 11992); III et al. (1993) Biophys J. 64:919; Loetscher et al. (1992) J. Chromatography 595:113-199; U.S. Pat. No. 5,443,816; Hale (995) Analytical Biochem. 231:46-49).

Other suitable methods for linking molecules and biological particles to solid supports are well known to those of skill in this art (see, e.g., U.S. Pat. No. 5,416,193). These linkers include linkers that are suitable for chemically linking molecules, such as proteins and nucleic acid, to supports include, but are not limited to, disulfide bonds, thioether bonds, hindered disulfide bonds, and covalent bonds between free reactive groups, such as amine and thiol groups. These bonds can be produced using heterobifunctional reagents to produce reactive thiol groups on one or both of the moieties and then reacting the thiol groups on one moiety with reactive thiol groups or amine groups to which reactive maleimido groups or thiol groups can be attached on the other. Other linkers include, acid cleavable linkers, such as bismaleimideothoxy propane, acid labile-transferrin conjugates and adipic acid diihydrazide, that would be cleaved in more acidic intracellular compartments; cross linkers that are cleaved upon exposure to UV or visible light and linkers, such as the various domains, such as C_{H}1, C_{H}2, and C_{H}3, from the constant region of human IgG₁ (see, Batra et al. (1993) Molecular Immunol. 30:379-386).

Presently preferred linkages are direct linkages effected by adsorbing the molecule or biological particle to the surface of the support. Other preferred linkages are photocleavable linkages that can be activated by exposure to light (see, e.g., Baldwin et al. (1995) J. Am. Chem. Soc. 117:5588; Goldmacher et al. (1992) Bioconj. Chem. 3:104-107, which linkers are herein incorporated by reference). The photocleavable linker is selected such that the cleaving wavelength that does not damage linked moieties. Photocleavable linkers are linkers that are cleaved upon exposure to light (see, e.g., Hazum et al (1981) in Pept., Proc. Eur. Pept. Symp., 16th, Brunfeldt, K (Ed), pp. 105-110, which describes the use of a nitrobenzyl group as a photocleavable protective group for cysteine; Yen et al. (1989) Makromol. Chem 190:69-82, which describes water soluble photocleavable copolymers, including hydroxypropylmethacrylamide copolymer, glycine copolymer, fluorescein copolymer and methylrhodamine copolymer; Goldmacher et al. (1992) Bioconj. Chem. 3:104-107, which describes a cross-linker and reagent that undergoes photolytic degradation upon exposure to near UV light (350 nm); and Senter et al. (1985) Photochem. Photobiol 42:231-237, which describes nitrobenzyloxycarbonyl chloride cross linking reagents that produce photocleavable linkages). Other linkers include fluoride labile linkers (see, e.g., Rodolph et al. (1995) J. Am. Chem. Soc. 117:5712), and acid labile linkers (see, e.g., Kick et al. (1995) J. Med. Chem. 38:1427)). The selected linker depends upon the particular application and, if needed, may be empirically selected.

F. Use of the Methods for Identification of Proteins of Desired Properties from a Library

1. Arraying Capture Agents

The capture agent molecules to which the epitope tags specifically bind are linked to supports, such as identifiable beads, such as microsheres, or solid surfaces. Linkage can be effected through any suitable bond, such as ionic, covalent, physical, van de waals bonds. It can be effected directly or via a suitable linker. For exemplary purposes arraying on surfaces is described.

Purified antibodies (1 μl at a concentration of 1-2 mg/ml in a buffer of 0.1 M PBS (phospahte buffered saline, pH 7.4) on glycerol (1-20% vol/vol), are spotted onto a membranes (such as; UltraBind membrane, Pall Gelman; FAST nitrocellulose coated slides, Schleicher & Schuell), chemically deactivated glass slides, superaldehyde slides (Telechem), polylysine coated glass, activated glass, or specific thin films and self-assembled monolayers International PCT application Nos WO 00/04389, WO 00/04382 and WO 00/04390). using an automated arraying tool (such as systems available from, for example, Microsys; PixSys NQ; Cartesian Technologies; BioChip Arrayer; Packard Instrument Company; Total Array System; BioRobotics; Affymetrix 417 Arrayer; Affymetrix, and others). The spots are allowed to air dry for a suitable period of time, 1-2 minutes or more, typically 30 min to 1 hr. Two membrane attachments are described. The UltraBind membrane (Pall Gelman) contains active aldehyde groups that react with primary amines to form a covalent linkage between the membrane and the capture agent, such as an antibody. Unreacted aldehydes are blocked by incubation with suitable blocking solution, such as a solution of 50 mM PBS, pH 7.4, 2% bovine serum albumin (BSA) or with BBSA-T (a protein-containing solution such as Blocker BSA “(Pierce) diluted to 1× in phosphate-buffered saline (PBS) with Tween-20 (polyoxyethylenesorbitan monolaurate; Sigma) added to a final concentration of 0.05% (vol:vol)) for a suitable time, such as about 30 minutes. The filter can be rinsed with PBS.

Capture agents, such as antibodies, also can be deposited onto membranes, such as, for example, nitrocellulose paper (Schliecher& Schuell) with, for example, an inject printer (i.e., Canon model BJC 8200, color inject printer), modified for this use and connected to a computer, such as a personal computer (PC). Such modifications, include, removal of the color ink cartridges from the print head and replacement with, for example, 1 milliliter pipette tips, which are hand-cut to fit in a sealed manner over the the inkpad reservoir wells in the print head. Antibody solutions are pipetted into the pipette tips reservoirs that are seated on the inkpaad reservoirs.

Printed images, using the modified printer, are generated, with, for example, Microsoft PowerPoint. The images are then printed onto nitrocellulose paper, which is cut to fit and then taped over the center of a sheet of printing paper. The set of papers is then fed into the printer immediately prior to printer.

Purified capture agents, such as antibodies can also be spotted onto FAST nitrocellulose coated slides, (Schleicher & Schuell). Nitrocellulose binds proteins by noncovalent adsorbtion. Nitrocellulose binds approximately 100 μg per cm². After binding of the capture agents, such as antibodies, remaining binding sites are blocked by incubation with a solution of 50 mM PBS, pH 7.4, 2% bovine serum albumin (BSA) or BBSA-T for a suitable time, such as for 30 minutes.

Direct binding of antibodies to the nitrocellulose results in non-oriented binding. The percentage of active immobilized antibody molecules can be increased by binding to nitrocellulose that has been coated with an antibody capture protein (such as protein A, protein G or anti-IgG monoclonal antibody). The antibody capture proteins arebound to the nitrocellulose before application of the library proteins, such as tagged antibodies, with an arrayer. Biotinylated antibodies can also be printed onto surfaces coated with avidin or strepavidin. The size and spacing of the spots can be adjusted depending on the filter used and the sensitivity of the assay. Typical spots are about 300-500 μm in diameter with 500-800 μm pitch.

Antibodies can also be printed onto activated glass substrates. Prior to printing the glass is cleaned ultrasonically in succession with a 1:10 dilution of detergent in warm tap water for 5 minutes in Aquasonic Cleaning Solution (VWR), multiple rinses in distilled water and 100% methanol (HPLC grade) followed by drying in a class 100 oven at 45° C. Clean glass is chemically functionalized by immersion in a solution of 3-aminopropyltriethoxysilane (APTS) (5% vol/vol in absolute ethanol) for 10 minutes. The glass is then rinsed in 95% ethanol, allowed to air dry, and then heated to 80° C. in a vacuum oven for 2 hours to cure. The surface can then be further modified to bind primary amines or free sulfhydryl groups in the antibody or avidin or strepavidin linked to the antibody with biotin. To create an amine-reactive surface, the functionalized glass is treated with a solution of Bis[sulfosuccinimidyl]suberate (BS³)(5 mg/ml in PBS, pH 7.4) for 20 minutes at room temperature. The N-hydroxysuccinimide (NHS)-activated glass surface is rinsed with distilled water and placed in a 37° C. dust-free class 100 oven for 15 minutes to dry. Antibodies can be directly attached to this surface or the surface can be coated with a protein such as protein A that binds the antibodies, protein G or anti-lgG monoclonal antibody or avidin/strepavidin, to bind biotinylated proteins. To create a sulfhydryl-reactive surface, the functionalized glass is treated with a solution of sulfosuccinimidyl 4-[N-maleimidomethyl]-cyclohexane-1-carboxylate (Sulfo-SMCC) for 20 minutes at room temperature. The maleimide-activated glass surface is rinsed with distilled water and placed in a 37° CC dust-free class 100 oven for 15 minutes to dry. To create a biotinylated surface, the functionalized glass is treated with a solution of EZ-link Sulfo-NHS-LC-Biotin (Pierce) for 20 minutes at room temperature. The biotinylated glass surface is rinsed with distilled water and placed in a 37° C. dust-free class 100 oven for 15 minutes to dry. The same immobilization strategies described above also can be used in self-assembled monolayers formed on top of inorganic thin films.

2. Exemplary use for Identification of a Genes from a Library of Mutated Genes

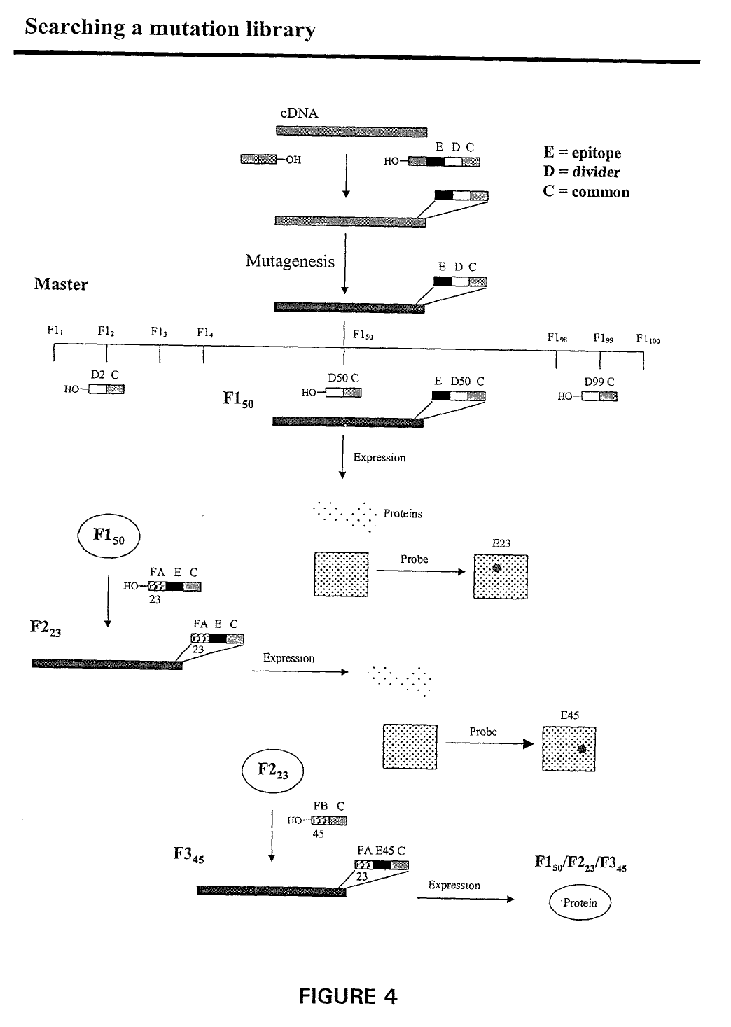

FIG. 4 illustrates the use of the methods herein to search a library of mutated genes. Mutation of specific gene regions by a variety of methods is often used to improve the properties of proteins encoded by the mutated genes, such as mutated genes produces by error-prore PCR or gene shuffling mutagenesis techniques to improve the binding affinity of a recombinant antibody. This technique coupled with selection by surface display has been used to improve the binding affinities of antibodies by several orders of magnitude. Mutation has also been used to improve the catalytic properties of enzymes. The methods herein provide means to screen and identify mutated genes encoding proteins having desired properties.

Initially a set of oligonucleotides containing various functional domains are added to the 3′ ends of a gene to be mutated by incorporation of a primer that contains sequences of nucleoties that hybridize to the gene and also additional sets of sequences, designated E for “Epitopes” D for “Divider”, and C for “Common”). The E D C sequences constitute sets of sequences, each defined by the functions in the nucleic acid. As noted, the E sequences encode the epitopes specifically recognized by antibodies in the collection. They are incorporated in-frame with the coding sequences of the gene to be! mutated and are expressed as a fusion with the parent protein. The D sequences are unique sequence sets downstream from the epitopes. They serve as specific priming sites to “Divide” the master group. They can be non-coding sequences and do not necessarily end up being part of the expressed mutated proteins. The C sequence is a sequence “Common” to all of the genes and provides a means for simultaneous PCR amplification of all the gene templates. As noted previously, in certain embodiments the D and/or C sequences are optional. Importantly, the E and D sequences are randomly distributed among the resulting DNA molecules. For example, 100 E sequences and 100 D sequences combine to create 10,000 (100×100=10,000) uniquely tagged cDNA molecules. Likewise, 1,000 E sequences and 1,000 D sequences combine to create 1,000,000 (1,000×1,000=1,000,000) uniquely tagged cDNA molecules.

Before, or after the E C and D sequences have been added to the ends of the molecule to be mutated, defined regions within the gene are mutated by a variety of standard methods. The mutation procedure should not produce mutations in the E D C sequences. After the mutagenesis has been completed, the mutated DNA is added as template to a first set of PCR reactions to create the F1 sublibrary. In addition to the template DNA, D C primer sets are separately added such that each PCR contains a primer complementary to a different D sequence. For example, in FIG. 4 the second PCR tube is identical to the rest of the tubes except it contains a D C primer containing only one of the 100 D sequences (D₂). In this illustration, tube 50 is identical to the rest of the Fl reaction tubes except it contains a different one of the 100 D sequences (D₅₀). The resulting PCR amplification products contain all of the 100 different E sequences randomly distributed among the genes but only containing one of the 100 D sequences. In the illustration, PCR tube 50 produces a sublibrary DNA molecules (F1₅₀) that all have the same D₅₀ sequences, the same C sequence but different E sequences randomly distributed among the molecules (ED₅₀ C).

The generated Fl DNA molecules are expressed in vitro using a transcription-translation extract. Appropriate regulatory DNA sequences, including promoters, ribosome binding sites and other such regulatory sequences known to those of skill in the art, for efficient in vitro transcription and translation are incorporated into the DNA fragments during the tagging process. As illustrated in FIG. 4, expression of the F1₅₀ DNA molecules produces a collection of proteins containing the various epitope tags. Proteins produced in bacteria or in other in vivo systems also can be used.

The resulting expressed proteins are incubated with the antibody collection, such as in an array format under conditions that permit binding between the epitopes and the antibody(ies) specifically selected to bind to each of the epitopes. This results in specific binding of proteins to antibodies. If the antibodies are arranged in an array, this results in the distribution of the tagged proteins to locations on the array containing immobilized antibodies that bind the proteins cognate epitopes.

After binding, the array is washed, probed, and analyzed by any method known to those of skill in the art, such as by enzymatic labeling, such as with luciferase. For example, analysis can be effected by photon collection using detectors, such as a photomultiplier tube, a photodiode array or preferably charge coupled device (CCD)-based imaging detector to detect emitted light. Photons can be produced by local enzymatic chemiluminescent, particularly bioluminescent reactions. Photon collection is preferred, since it advantageously is relatively inexpensive, very sensitive and the sensitivity can be amplified by increased collection times.

As an example, if the search is used to identify mutations to the luciferase enzyme that confer increased activity, the array is washed, bathed in substrate and then analyzed for increased luciferase activity as measured by increased photon output. The “brightest spot” in the array has bound the enzyme with the most favorable mutations.

As another example, if the search is used to identify increased affinity of an antibody for its antigen, the array is washed then incubated with tagged antigen. The tag on the antigen is used to bind to a secondary detection reagent such as strepavidin conjugated HRP if the antigen is tagged with biotin, or an antibody-HRP complex, if the tag is a defined epitope. Again, the “brightest spot” contains the mutant antibody with the greatest affinity, having bound the greatest amount of antigen. Knowing the location of the “brightest spot” and epitope binding specificity of the antibodies in that spot, identifies the E sequence associated with the mutant gene of interest. At this point in the sort, the template for the gene of interest (as illustrated in FIG. 4) is known to be in the F1₅₀ sublibrary and contain the E23 sequence (F1₅₀/F2₂₃).

Genes containing the E23 sequence can be amplified using template DNA from the F1₅₀ sublibrary and PCR primers with sequences corresponding to the E23 sequence (FA₂₃ E C). Like the D C set of primers used to initially divide the master library, the FA E C set of primers are used to amplify templates containing specific E sequences and at the same time re-distribute E sequences among the amplified genes. The FA E C primer is composed of 3 functional regions. The FA region contains sequences corresponding to an upstream fragment (Fragment A) of the E sequence present in the template. The FA region contains any amount of the E sequence that confers hybridization specificity, but that, upon translation, does not confer the epitope binding specificity. As before, the E region encodes epitope sequences and the C region encodes a common sequence for amplification. The FA and E sequences are in-frame with the coding region of the gene. The resulting amplified genes represent an F2 sublibrary (F2₂₃).

The amplified genes from the F2 sublibrary are expressed in vitro, incubated with the antibody array, re-probed and analyzed. As before, “bright spots” in this array identifies the E sequence associated with the mutant gene of interest. At this point in the sort, the gene of interest (as illustrated in FIG. 4) is known to be in the F1₅₀ and F2₂₃ sublibrarys and contains the E45 sequence (F1₅₀/F2₂₃/F3₄₅). This information identifies a specific gene that can be amplified using a primer specific for the E45 sequence (FB₄₅C). The FB C primer is composed of two functional regions. The FB region contains sequences corresponding to a downstream fragment (Fragment B) of the E sequence present in the template. FB can contain all or part of E; C is optional. FB contains any part, up to and including all of the E encoding sequence, to confer hybridization specificity. As before, the C region encodes a common sequence for amplification. The resulting amplified genes represent an F3 sublibrary (F3₄₅).

G. Identification of Recombinant Antibodies

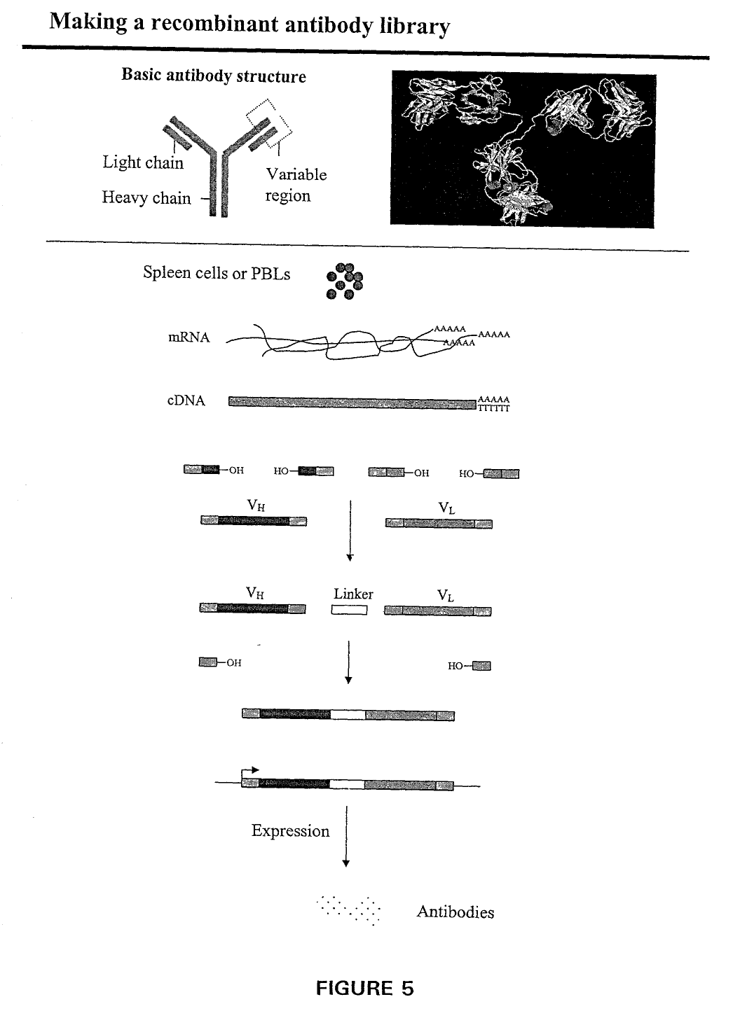

Another application of the technology is its use for the identification of recombinant antibodies. Antibodies with desired properties are sorted out of large pools of recombinant antibody genes. An overview of a standard method for constructing recombinant antibody libraries is illustrated in FIG. 5. The initial steps involve cloning recombinant antibody genes from mRNA isolated from spleenocytes or peripheral blood lymphocytes (PBLs). Functional antibody fragments can be created by genetic cloning and recombination of the variable heavy (V_{H}) chain and variable light (V_{L}) chain genes. The V_{H} and V_{L} chain genes are cloned by first reverse transcribing mRNA isolated from spleen cells or PBLs into cDNA. Specific amplification of the V_{H} and V_{L} chain genes is accomplished with sets of PCR primers that correspond to consensus sequences flanking these genes. The V_{H} and V_{L} chain genes are joined with a linker DNA sequence. A typical linker sequence for a single-chain antibody fragment (scFv) encodes the amino acid sequence (Gly₄Ser)₃. After the V_{H}-linker-V_{L} genes have been assembled and amplified by PCR, the products can be transcribed and translated directly or cloned into an expression plasmid and then expressed either in vivo or in vitro to produce functional recombinant antibody fragments.

The method of recombinant antibody library construction can be adapted for use with the sorting methods herein. This is accomplished by incorporating the E D C sequences into the V_{L} chain genes before assembly with the V_{H} chain and linker sequences. After the recombinant antibody library has been tagged with the E D C sequences, it is sorted by division into the F1 sublibrarys followed by screening with the arrays as described above.

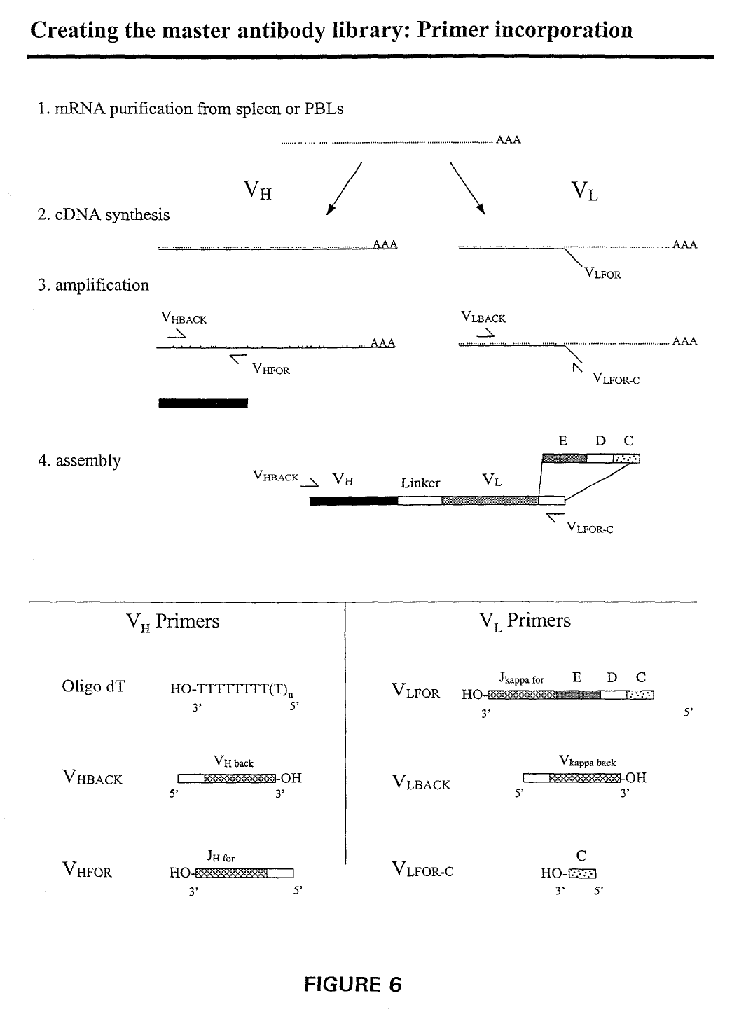

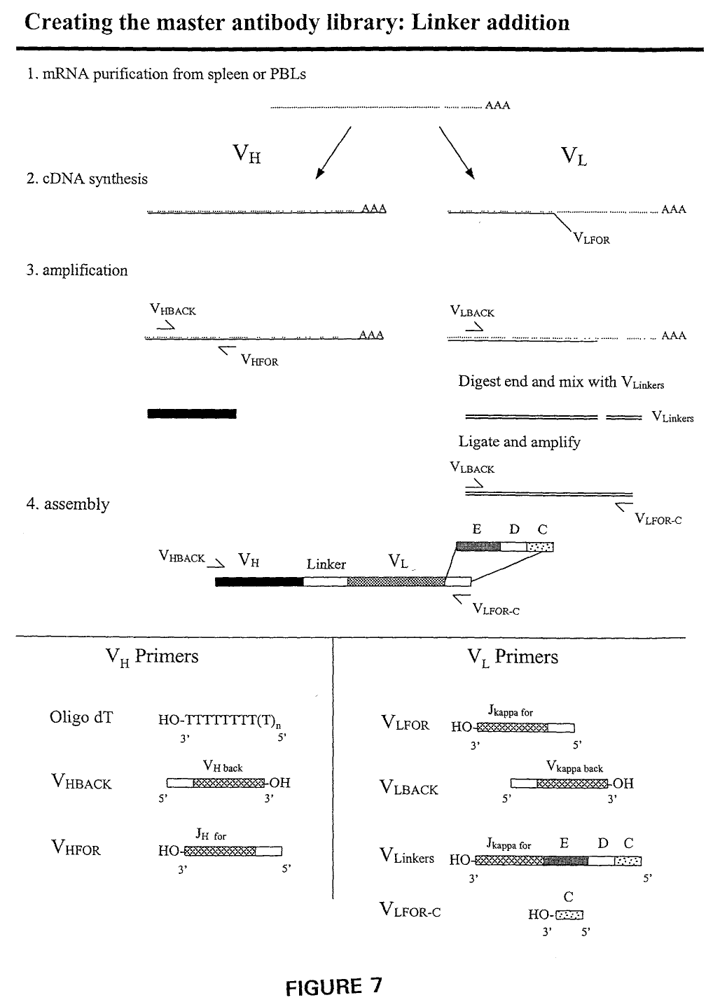

Two different methods are illustrated for incorporating the E D C sequences into the amplified V_{L} chain genes. In the first method, the E D C sequences are part of the first-strand cDNA synthesis primer and get incorporated during cDNA synthesis (FIG. 6) in the second method the E D C sequences are incorporated after cDNA synthesis (FIG. 7) by the addition of double-stranded DNA linker molecules.

FIG. 6 illustrates how E D C sequences are put onto the V_{L} chain genes by primer incorporation. The V_{H} chain genes are cloned using standard methods. The mRNA isolated from spleen cells or PBLs is converted to cDNA using a universal oligo dT primer or IG gene-specific primers. The V_{H} genes are then specifically amplified using a set of primers that are complementary to consensus sequences that flank these genes. The V_{HBACK} primer also contains promoter sequences that are required for in vitro transcription and translation of the assembled gene. and/or allows subcloning into plasmid vectors for in vivo expression in cells, such as, but are not limited to, bacterial, yeast, insect and mammalian cells.

The V_{L} gene is cloned using a set of reverse transcription primers (V_{L}FOR) that contain sets of sequences that are complementary to downstream consensus sequences flanking the V_{L} genes (J_{kappafor}) and the E D C sequences. The E D C sequences are located 5′ to the Jₖₐₚₚₐ for sequences in the V_{LFOR} primer. The second strand of the cDNA is primed using an oligonucleotide (V_{LBACK}) containing complementary sequences to the upstream consensus region of the V_{L} gene (V_{kappa back}) After the second strand cDNA synthesis the V_{L} genes are amplified with a combination of the V_{LBACK} and V_{LFOR-C} primers. The V_{LFOR-C} primer consists of sequences complementary to the C region of the E D C sequence.

After amplification of the V_{H} and V_{L} genes the fragments are digested with a restriction enzyme to produce overlapping ends with the linker. The V_{H}-linker-V_{L} fragments are sealed with DNA ligase and then amplified using the V_{HBACK} and V_{LFOR-C} primers.

In the second method, illustrated in FIG. 7, the V_{H} genes are amplified as described above. This method differs from the first in that the V_{L} gene first-strand synthesis is primed with an oligonucleotide containing a unique restriction site 5′ to the J_{kappa for} sequences. This restriction site is incorporated into the 3′-end of the resulting cDNA such that a unique cohesive end can be produced by restriction enzyme digestion. The linkers are mixed with the cut cDNA, sealed with ligase and then amplified with a combination of the V_{HBACK} and V_{LFOR-C} primers.

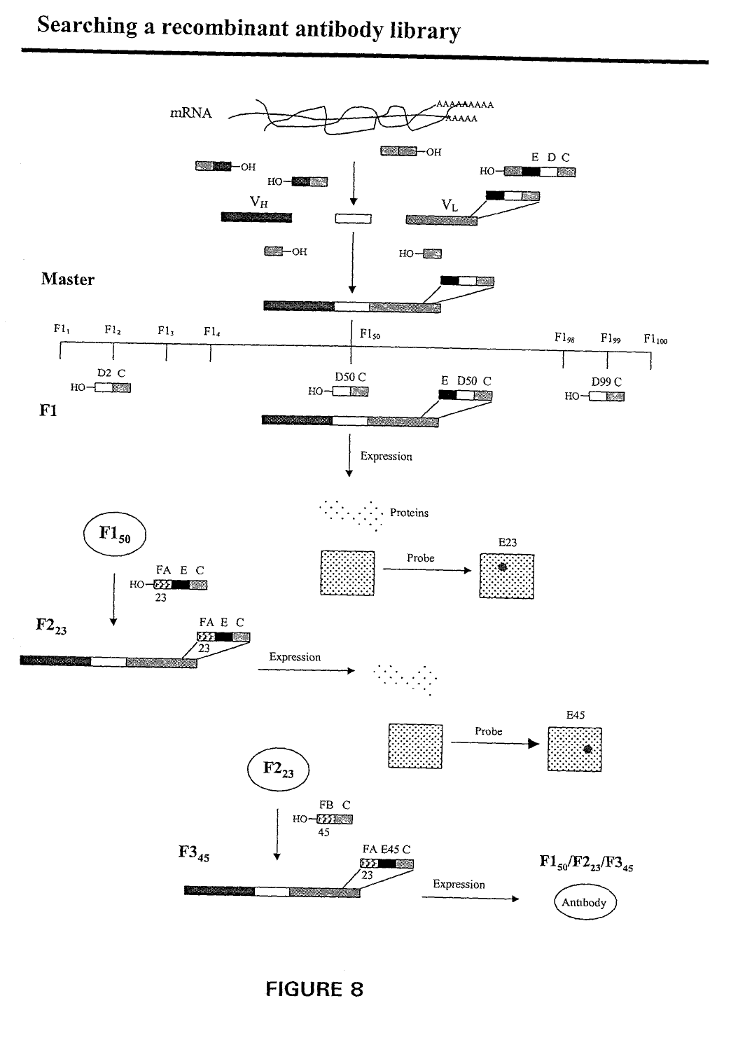

FIG. 8 outlines a method for searching a recombinant antibody library. The V_{H} and V_{L} genes are cloned as described above and the E D C sequences are added to the 3′-end of the antibody genes to create the master library. The F1 sublibrarys are created using the D C set of PCR primers. The illustration depicts 100 F1 sublibrarys, shows D C primers for F1₂, F1₅₀ and F1₉₉, and shows the amplified product from the F1₅₀ reaction.

Transcription and translation of the F1₅₀ sublibrary genes produces a variety of recombinant capture agents, such as antibodies, that can be randomly grouped according to the epitopes (E sequences) they contain. The expressed proteins are bathed over the array and allowed to sort onto spots in the array that contain antibodies that bind their specific epitope tags. After the scFvss from sublibrary F1₅₀ are bound to the array, labeled antigen is bathed over the array. The label on the antigen can be a chemical tag, such as biotin, used to bind a secondary detection reagent such as strepavidin conjugated HRP, or the antigen can be epitope tagged and detection achieved with an anti-epitope antibody-HRP complex. After binding, the array is washed, probed, and analyzed. Analysis is typically by photon collection using a CCD-based imaging detector and photons are typically produced by local enzymatic chemiluminescent reactions. Again, the “brightest spot” contains the recombinant antibody with the greatest affinity having bound the greatest amount of antigen.

Knowing the location of the “brightest spot” and epitope binding specificity of the antibodies in that spot, identifies the E sequence associated with the recombinant antibody gene of interest. At this point in the sort, the template for the gene of interest (as illustrated in FIG. 8) is known to be in the F1₅₀ sublibrary and contain the E23 sequence.

Genes containing the E23 sequence can be amplified using template DNA from the F1₅₀ sublibrary and PCR primers with sequences corresponding to the E23 sequence (FA₂₃ E C). Like the D C set of primers used to initially divide the master library, the FA E C set of primers are used to amplify templates containing specific E sequences and at the same time re-distribute E sequences among the amplified genes. The FA₂₃ E C primer is used to amplify template DNA from the F1₅₀ sublibrary. The resulting amplified genes represent an F2 sublibrary, F2₂₃. The initial lineage for the antibody of interest is F1 ₅₀/F2₂₃.

The amplified genes from the F2 sublibrary are expressed in vitro or in in vivo systems, incubated with the antibody array, re-probed and analyzed. As previously, “bright spots” in this array identifies the E sequence associated with the recombinant antibody gene of interest. At this point in the sort, the gene of interest (as illustrated in FIG. 8) is known to be in the F1₅₀ and F2₂₃ sublibrarys and contains the E45 sequence (Fl ₅₀/F2₂₃/F3₄₅). This information identifies a specific gene that can be amplified using a primer specific for the E45 sequence (FB₄₅ C). The resulting amplified genes represent an F3 sublibrary (F3₄₅77) that contains a single type of recombinant antibody.

H. Detection of Bound Antigen(s)

Bound polyeptide-tagged molecules can be detected by any suitable method known to those of skill in the art and is a function of the target molecules. Exemplary detection methods include the use of chemiluminescence and bioluminescence generating reagents, such as horse radish peroxidase (HRP) systems and luciferin/luciferase systems, alkaline phosphaase (AP), labeled antibodies, fluorophores and isotopes. These can be detected using film, photon collection, scanning lasers, waveguides, ellipsometry, CCDs and other imaging means.

As noted, uses of the addressable anti-tag capture agent collections include, but are not limited to: searching a recombinant antibody scFv library to identify scFV includes, but is not limited to, finding single antigen or multiple antigens; searching mutation libraries, including tagging mutant libraries; mutation by error prone PCR; mutation by gene shuffling for searching for small molecule binders, searching for increased antibody affinity, searching for enhanced enzymatic properties (AP, HRP, Luciferase, GFP); searching for sequence-specific DNA binding proteins; searching a cDNA library for protein-protein interactions; and any other such application.

### I. EXAMPLES

The following examples are included for illustrative purposes only and are not intended to limit the scope of the invention.

### Example 1

Preparation of Anti-tag Antibody Collections

A. Generating a Collection of Antibody—Tag Pairs

A collection of antibodies that bind peptide tags is used to sort molecules linked to the tags. The collection of antibodies that specifically bind to the polypeptide tags can be generated by a variety of methods. Two examples are described below.

1. Hybridoma Screening

In the first example, high affinity and high specificity antibodies for the array are identified by screening a randomly selected collection of individual hybridoma cells against a phage display library expressing a random collection of peptide epitopes. The hybridoma cells are created by fusion of spleenocytes isolated from a naive (non-immunized) mouse with myeloma cells. After a stable culture is generated, approximately 10-30,000 individual cell clones (monoclonals) are isolated and grown separately in 96-well plates. The culture supernatants from this collection are screened by ELISA with an anti-IgG antibody to identify cultures secreting significant amounts of antibody. Cultures with low antibody production are discontinued. Antibodies from this monoclonal collection are separately affinity purified from culture supernatants using high throughput 96-well purification methods and the amounts purified and quantified.

The purified antibodies are arrayed by robitic spotting onto a filter and are also separately mixed then bound to paramagnetic beads to create a substrate for panning high affinity epitopes from a filamentous M13 bacteriophage library displaying random cysteine-constrained heptameric amino acid sequences. The phage library is enriched for phage displaying high affinity epitopes by mixing the phage library with the antibody-coated beads and washing away loosely-bound phage from the beads (“panning”). Several rounds of panning leads to a highly enriched library containing phage that tightly bind to the monoclonal antibodies present in the collection. To separate and identify high affinity phage-antibody pairs, the enriched phage library is incubated with the filter containing the arrayed antibodies under high stringency binding conditions. Phage bound to antibodies on the filter are identified by staining with HRP-conjugated anti-phage antibodies and a chemiluminescent substrate to produce a luminescent signal. The signal is quantified using a high resolution CCD camera imaging device. High affinity binding phage are recovered from the filter and propagated. Several independent phage clones recovered from each spot are sequenced to identify consensus high-affinity epitopes for the corresponding antibodies.

a. Making Hybridomas

Hybridoma cells are prepared by well known methods known to those of skill in the art (see, e.g., Harlow et al. (1988) Antibodies: A Laboratory Manual, Cold Spring Harbor Laboratory, Cold Spring Harbor). Hybridoma cells are created by the fusion of mouse spleenocytes and mouse myeloma cells. For the fusion, antibody-producing cells isolated from the spleen of a non-immunized mouse are mixed with the myeloma cells and fused. Alternatively, the hybridoma cells are created from spleenocytes isolated from a mouse previously immunized with a recombinant protein (e.g. dihydrofolate reductase, DHFR) containing a mixture of different epitope tags and conjugated to a carrier (i.e. Keyhole limpet hemocyanin, KLH). The epitope tags are random cysteine-constrained peptides expressed as part of a genetic fusion to the DHFR gene. The random peptides are encoded by a DNA insert assembled from synthetic degenerate oligonucleotides and cloned into the gene III protein (gIII) of the filamentous bacteriophage M13. DNA encoding the peptide library is available commercially (Ph.D.-C7C™ Disulfide Constrained Peptide Library Kit, New England Biolabs). The Ph.D.-C7C™ library contains approximately 3.7×10⁹ different peptides After fusion, cells are diluted into selective media and plated into multiwell tissue culture dishes. A healthy, rapidly dividing culture of mouse myeloma cells are diluted into 20 ml of medium containing 20% fetal bovine serum (FBS) and 2× OPI. Medium is typically Dulbecco's modified Eagle's (DME) or RPMI 1640 medium. Ingredients of mediums are well known (see, e.g., Harlow et al. (1988) Antibodies: A Laboratory Manual, Cold Spring Harbor Laboratory, Cold Spring Harbor). Antibody producing cells are prepared by aseptic removal of a spleen from a mouse and disruption of the spleen into cells and removal of the larger tissue by washing with 2× OPI medium. A typical mouse spleen contains approximately 5×10⁷ to 2×10⁸ lymphocytes. As the hybridomas being prepared are not enriched by immunization to any antigen, spleens from more than one mouse can be used and the cells mixed. Equal numbers of spleen cells and myeloma cells are pelleted by centrifugation (400× g for 5 min) and the pellets separately resuspended 5 ml of medium without serum and then combined. Polyethylene glycol (PEG) is added to 0.84% from a 43% solution. The cells are gently resuspended in the PEG-containing medium and then repelleted by centrifugation at 400× g for 5 minutes, washed by resuspension in 5 ml of medium containing 20% FBS, repelleted and washed a second time in medium supplemented with 20% FBS, 1× OPI, and 1× AH (AH is a selection medium; 1× AH contains 5.8 μM azaserine and 0.1 mM hypoxanthine). Cells are incubated at 37° C. in a CO₂ incubator. Clones should be visible by microscopy after 4 days.

b. Isolating Hybridoma Cells

Stable hybridomas are selected by growth for several days in poor medium. The medium is then replaced with fresh medium and single hybridomas are isolated by limited dilution cloning. Because hybridoma cells have a very low plating efficiency, single cell cloning is done in the presence of feeder cells or conditioned medium. Freshly isolated spleen cells can be used as feeder cells as they do not grow in normal tissue culture conditions and are lost during expansion of the hybridoma cells. In this procedure a spleen is aspectically removed from a mouse and disrupted. Released cells are washed repeatedly in medium containing 10% FBS. A spleen typically produces 100 ml of 106 cells per ml. The feeder cells are plated in 96-well plates, 50 μl per well, and grown for 24 hrs. Healthy hybridoma cells are diluted in medium containing 20% FBS, 2× OPI to a concentration of 20 cells per ml. Cells should be as free of clumps as possible. Add 50 μl of the diluted hybridoma cells to the feeder cells, final volume is 100 μl. Clones begin to appear in 4 days. Alternatively single cells can be isolated by single-cell picking by individually pipetting single cells and then depositing in wells containing feeder cells. Single cells can also be obtained by growth in soft agar. Once healthy, stable cultures are achieved the cells are maintained by growth in DME (or RPMI 1640) medium supplemented with 10% FBS. Stable cells can be stored in liquid nitrogen by slow freezing in medium containing a cryoprotectant such as dimethylsulfoxide (DMSO). The amount of antibody being produced by the cells is determined by measuring the amount of antibody in the culture supernatants by the ELISA method.

2. Purification of Antibodies from Hybridoma Culture Supernatants

Purification of antibodies from the individual culture supernatants is achieved by affinity binding. A number of affinity binding substrates are available. The procedure described below is based on commercially available substrates containing immobilized protein L (Pierce) and follows the manufacturers suggested procedure. Briefly, dilute the culture supernatant 1:1 with Binding buffer (0.1 M phosphate, 0.15 M sodium chloride (NaCl), pH 7.2) and apply up to 0.2 ml of the diluted sample to a Reacti-Bind™ Protein L Coated plate (Pierce) pre-equilibrated with Binding buffer. Wash the wells with 3×0.2 ml of binding buffer. Elute the bound antibodies with 2×0.1 ml of Elution buffer (0.1 M glycine, pH 2.8) and combine with 20 μl of 1 M Tris, pH 7.5. Desalt the purified antibodies using Sephadex G-25 gel filtration in combination with 96-well filter plates (NaIge Nunc).

To create the phage panning substrates, antibodies separately purified as described above can be combined. Alternatively, purified antibody mixtures can be obtained by batch purification from pooled culture supernatants. Purification of antibodies from the pooled culture supernatants is also achieved by affinity binding. A number of affinity binding substrates are available. The procedure described below is based on commercially available substrates containing immobilized protein L (Pierce) and follows the manufacturers suggested procedure. Briefly, dilute the culture supernatant 1:1 with Binding buffer and apply up to 4 ml of the diluted sample to an Affinity Pack™ Immobilized Protein L Column (Pierce) pre-equilibrated with Binding buffer. Wash the column with 20 ml of Binding buffer, or until the absorbance at 250 nm has returned to background. Elute the bound antibodies with 6-10 ml of Elution buffer and collect into 1 ml fractions containing 100 μl of 1 M Tris, pH 7.5. Monitor release of bound proteins by absorbance at 280 nm and pool appropriate fractions. Desalt the purified antibodies using an Excellulose” Desalting Column (Pierce).

3. Arraying Antibodies onto Filters

The antibodies purified from individual hybridoma cultures are spotted onto a membrane (such as; UltraBind membrane, Pall Gelman; FAST nitrocellulose coated slides, Schleicher & Schuell) 1 μl at a concentration of 1 μg-1 mg/ml in a buffer of 0.1 M PBS (phospahte buffered saline), pH 7.4, using an automated arraying tool (such as; PixSys NQ nanoliter dispensing workstation, Cartesian Technologies; BioChip Arrayer; Packard Instrument Company; Total Array System; BioRobotics; Affymetrix 417 Arrayer; Affymetrix). The spots are allowed to air dry 1-2 minutes. The UltraBind membrane contains active aldehyde groups that react with primary amines to form a covalent linkage between the membrane and the antibody. Unreacted aldehydes are blocked by incubation with a solution of 50 mM PBS, pH 7.4, 2% bovine serum albumin (BSA) for 30 minutes. The filter can be rinsed with 50 mM PBS and then air dried completely.

4. Panning a Phage Display Library on Paramagnetic Beads

A phage library containing random cysteine-constrained peptides expressed as part of an N-terminal genetic fusion to the gene III protein (gIII) of the filamentous bacteriophage M13 is constructed essentially as decribed (Kay et al. (1996) Phage Display of Peptides and Proteins: A Laboratory Manual, Academic Press, San Diego). The random peptides are encoded by a DNA insert assembled from synthetic degenerate oligonucleotides and cloned into gill. These libraries are available commercially (Ph.D.-C7C™ Disulfide Constrained Peptide Library Kit, New England Biolabs). The Ph.D.-C7C™ library contains approximately 3.7×10⁹ independent clones.

Combine 2×10¹¹ phage virions from the Ph.D.-C7C™ library with 300 μg of the purified antibodies and 300 ng of the human IgG4 monoclonal antibody specific for the Fc domain of mouse IgG (Dynal; this monoclonal does not bind to human antibodies) to a final volume of 0.2 ml with TBST (50 mM Tris-HCl (pH 7.4), 150 mM NaCl, 0.1% Tween-20). The final concentration of antibody is approximately 10 nM. Incubate at room temperature for 20 minutes.

Combine the phage-antibody solution with Dynabeads Pan Mouse IgG (Dynal). The beads are supplied as a suspension in PBS, pH 7.4, 0.1% BSA, 0.02% sodium azide. The beads are washed with TBS (50 mM Tris-HCl (pH 7.4), 150 mM NaCl) several times prior to mixing with phage. The beads are separated from the solution by application of a magnet (Magnetic Particle Concentrator, Dynal). Add the phage-antibody solution to a concentration of 0.1 μg/10⁷ beads and incubate at 4° C. for 30 minutes with gentle tilting and rotation. Inclusion of the human antibody prevents selection of phage that bind to the human antibody immobilized on the Dynabeads. Additionally, inclusion of human proteins from a lysed human cell as a blocker will prevent the selection of phage epitopes also present in human cells. The selected antibody-phage pairs should not be competed with proteins naturally pesent in the samples to be tested.

In the next step of the method, remove the fluid using the magnet and resuspend the beads in a Wash buffer of 1 ml of TBST. Repeat wash step 10 times. After the last wash step, elute the captured phage by suspending the beads in 1 ml of 0.2 M glycine-HCl, pH 2.2, 1 mg/ml BSA and incubating for 10 minutes at room temperature before recovering the fluid. The pH of the recovered fluid is immediately neutralized with the addition of 0.15 ml of 1 M Tris, pH 9.1. A small aliquat of the eluate is titered by infecting ER2738 Escherichia coli (E. coli) cells on LB-Tet plates.

Amplify the eluate by the addition of 20 ml of a mid-log culture of ER2738 E. coli and continue to grow in LB-Tet for 4.5 hours. Separate phage virions from E. coli cells by centrifugation at 10,000 rpm, 10 minutes, and transfer to fresh tube. Repeat, transfering the upper 80% of the supernatant to a fresh tube. Concentrate the phage by the addition of ⅙ volume of PEG/NaCl (20% w/v polyethylene glycol-8000, 2.5 M NaCI) followed by precipitation overnight at 4° C. The phage are recovered by centrifugation at 10,000 rpm for 15 minutes and the pellet is resuspended in 1 ml of TBS. Re-precipitate the phage in a microcentrifuge tube with PEG/NaCl and resuspend the pellet in 0.2 ml TBS, 0.02% sodium azide. Microcentrifuge for 1 minute to remove any residual material. The supernatant is the amplified eluate. Titer the amplified eluate and repeat the panning as described above 3 times. With each round of panning and amplification, the pool of phage becomes enriched for phage that bind the antibodies. If the concentration of phage used as input is kept constant, an increase in the number of phage recovered should occur. Phage can be stored at 4° C. or diluted 1:1 with sterile glycerol and stored at −20° C.

5. Staining the Antibody Array with Phage

The filter containing arrayed antibodies prepared from individual culture supernatants is probed with the enriched phage library. This method is similar to standard Western blotting or Dot blotting procedures. Briefly, the blocked filter is re-hydrated in TBST, pH 7.4, 0.1% v/v Tween-20, 1 mg/ml BSA, and incubated for 1 hour at 4° C. Phage are added to a concentration of 2×10¹¹ phage/ml and incubated with the filter for 30 minutes at room temperature. The hybridization solution is recovered and the filter is washed extensively with Blocking solution (TBST, pH 7.4, 0.1% v/v Tween-20, 1 mg/ml BSA and soluble proteins from human cells). To the Blocking solution add HRP-conjugated anti-M13 antibody (available commercially from, for, example, Amersham) diluted 1:100,000 to 1:500,000 in blocking buffer from a 1 mg/ml stock concentration and incubate for 1 hour with gentle shaking. Wash the membrane at least 4 to 6 times with TBST. Completely wet the blot in SuperSignal West Femto Substrate Working Solution (Pierce) for 5 minutes. The filter can be imaged by exposure to autoradiographic film (Kodak) or imaged using an imaging device such as a phosphoimager (BioRad) or charged coupled device (CCD) camera (Alphalnnotech; Kodak).

6. Recovery of Phage from Filter and Sequencing the Epitopes

Phage can be recovered from the filter by cutting out the spots containing phage identified from the imaging. Phage are eluted from the filter by suspending the filter piece in 0.5 ml of 0.2 M glycine-HCl, pH 2.2, 1 mg/ml BSA and incubating for 10 minutes at room temperature before recovering the fluid. The pH of the recovered fluid is immediately neutralized with the addition of 0.075 ml of 1 M Tris, pH 9.1. A small aliquat of the eluate is titered by infecting ER2738 E coli cells on LB-Tet plates. Isolated plaques (typically 10 plaques) are picked for DNA isolation and sequenced to define a consensus epitope. Plaques are amplified by inoculating 1 ml cultures of ER2738 E. coli cells freshly diluted 1:100 from a healthy mid-log culture, using a sterile pipet tip or toothpick and incubated at 37° C. for 4 to 5 hours with shaking. Phage are recovered by microcentrifugation for 30 seconds, and 0.5 ml of the supernatant transferred to a fresh tube and 0.2 ml of PEG/NaCl is added and allowed to stand at room temperature after gentle mixing for 10 minutes. Pellet the phage by centrifugation for 10 minutes at top speed in a microcentrifuge. Discard any remaining supernatant and thoroughly suspend the pellet in 0.1 ml iodine buffer and 0.25 ml ethanol to precipitate single-stranded DNA. The DNA pellets are washed in 70% ethanol and air-dried. DNA is sequenced by standard methods.

B. Selective Infection

Selective infection technologies, such as phage display, are used to identify interacting protein-peptide pairs. These systems take advantage of the requirement for protein-protein interactions to mediate the infection process between a bacteria and an infecting virus (phage). The filamentous M13 phage normally infects E.coli by first binding to the F pilus of the bacteria. The virus binds to the pilus at a distinct region of the F pilin protein encoded by the traA gene. This binding is mediated by the minor coat protein (protein 3) on the tip of the phage. The phage binding site on the F pilin protein (a 13 amino acid sequence on the traA gene) can be engineered to create a large population of bacteria expressing a random mixture of phage binding sites.

The phage coat protein (protein 3) can also be engineered to display a library of diverse single chain antibody structures. Infection of the bacteria and internalization of the virus is therefore mediated by an appropriate antibody-peptide epitope interaction. By placing appropriate antibiotic resistance markers on the bacteria and virus DNA, individual colonies can be selected that contain both genes for the antibody and its corresponding peptide epitope. The recombinant antibody phage display library prepared from non-immunized mice and the bacterial strains containing a random peptide sequence in the phage binding site in the traA gene are commercially available (Biolnvent, Lund, Sweden). Creation of a recombinant antibody library is described below.

C. Expression and Purification of Antibodies

Purification of antibodies from hybridoma supernatants is achieved by affinity binding. A number of affinity binding substrates are available. The procedure described below is based on commercially available substrates containing immobilized protein L (Pierce) and follows the manufacturers suggested procedure. Briefly, dilute the culture supernatant 1:1 with Binding buffer (0.1 M phosphate, 0.15 M sodium chloride (NaCI), pH 7.2) and apply up to 4 ml of the diluted sample to an Affinity Pack™ Immobilized Protein L Column (Pierce) pre-equilibrated with Binding buffer. Wash the column with 20 ml of Binding buffer, or until the absorbance at 250 nm has returned to background. Elute the bound antibodies with 6-10 ml of Elution buffer (0.1 M glycine, pH 2.8) and collect into 1 ml fractions containing 100,μl of 1 M Tris, pH 7.5. Monitor release of bound proteins by absorbance at 280 nm and pool appropriate fractions. Desalt the purified antibodies using an Excellulose™ Desalting Column (Pierce). The purification can be scaled as appropriate. Alternatively, antibodies can be purified by affinity chromatography using protein A (or protein G) HiTrap columns (Amersham Pharmacia) and an FPLC chromatographic system (Amersham Pharmacia). Following the manufacturers suggested protocols.

Recombinant antibodies are expressed and purified as described (McCafferty et al. (1996) Antibody engineering: A practical Approach, Oxford University Press, Oxford). Briefly, the gene encoding the recombinant antibody is cloned into an expression plasmid containing an inducible promoter. The production of an active recombinant antibody is dependent on the formation of a number of intramolecular disulfide bonds. The environment of the bacterial cytoplasm is reducing, thus preventing disulfide bond formation. One solution to this problem is to genetically fuse a secretion signal peptide onto the antibody which directs its transport to the non-reducing environment of the periplasm (Hanes et al. (1997) Proc. Natl. Acad. Sci. U.S.A. 94:4937-4942).

Alternatively, the antibodies can be expressed as insoluble inclusion bodies and then refolded in vitro under conditions that promote the formation of the disulfide bonds. Inoculate 0.5 liters of LB medium containing an appropriate antibiotic and shake for 10 hours at 32° C. Use the starter culture to inoculate 9.5 liters of production medium (3 g ammonium sulfate, 2.5 g potassium phosphate, 30 g casein, 0.25 g magnesium sulfate, 0.1 mg calcium chloride, 10 ml M-63 salts concentrate, 0.2 ml MAZU 204 Antifoam (Mazer Chemicals), 30 g glucose, 0.1 mg biotin, 1 mg nicotinamide, appropriate antibiotic, per liter, pH 7.4). Ferment using a Chemap (or like) fermenter at pH 7.2, aeration at 1:1 v/v Air to medium per minute, 800 rpm agitation, 32° C. When the absorbance at 600 nm reaches 18-20, raise temperature to 42° C. for 1 hour then cool to 10° C. for 10 minutes before harvesting cell paste by centrifugation at 7,000× g for 10 minutes. Recovery is typically 200-300 g wet cell paste from a 10 liter fermentation and should be kept frozen.

The recombinant antibody is solubilized from the thawed cell paste by resuspension in 2.5 liters cell lysis buffer (50 mM Tris-HCl, pH 8.0, 1.0 mM EDTA, 100 mM KCl, 0.1 mM phenylmethylsulfonyl fluoride; PMSF) and kept at 4° C. The resuspended cells are passed through a Manton-Gaulin cell homogenizer 3 times and the insoluble antibodies recovered by centrifugation at 24,300× g for 30 minutes at 6° C. The pellet is resuspended in 1.2 liters of cell lysis buffer and the homogenization and recovery is repeated as described above 5 times. The washed pellet can be stored frozen. The recombinant antibody is renatured by resolubilization in 6 ml denaturing buffer (6 M guanidine hydrochloride, 50 mM Tris-HCl, pH 8.0, 10 mM calcium chloride, 50 mM potasium chloride) per gram of cell pellet. The supernatant from a centrifugation at 24,300× g for 45 minutes at 6° C. is diluted to optical density of 25 at 280 nm with denturing buffer and slowly diluted into cold (4-10° C.) refolding buffer (50 mM Tris-HCl, pH 8.0, 10 mM calcium chloride, 50 mM potassium chloride, 0.1 mM PMSF) until a 1:10 dilution is achieved over a 2 hour period. The solution is left to stand for at least 20 hours at 4° C. before filtering through a 0.45 um microporous membrane. The filtrate is then concentrated to about 500 ml before final purification using an HPLC.

The filtrate is dialyzed against HPLC buffer A (60 mM MOPS, 0.5 mM calcium acetate, pH 6.5) until the conductivity matches that of HPLC buffer A. The dialyzed sample (up to 60 mg) is loaded onto a 21.5 mm×150 mm polyaspartic acid PolyCAT column, equilibrated with HPLC buffer A and eluted from the column with a 50 minute linear gradient between HPLC buffers A and B (HPLC buffer B is 60 mM MOPS, 0.5 mM calcium acetate, pH 7.5). Remaining protein is eluted with HPLC buffer C (60 mM MOPS, 100 mM calcium acetate, pH 7.5). The collected fractions are analyzed by SDS-PAGE.

D. Exemplary Array and use Thereof for Capture of Proteins with Epitope Tags and Detection Thereof

As also described in EXAMPLE 6, to demonstrate the functioning of the methods herein, capture antibodies, specific, for example, for various peptide epitopes, such as human influenza virus hemagglutinin (HA) protein epitope, which has the amino acid sequence YPYDVPDYA, are used to tag, for example, scFvs. For example, an scFv with antigen specificity for human fibronectin (HFN) is tagged with an HA epitope, thus generating a molecule (HA-HFN), which is recognized by an antibody specific for the HA peptide and which has antigen specificity of HFN.

After depositing the capture antibodies, including anti-HA tag capture antibodies onto a membrane, such as a nitrocellulose membrane, they are dried at ambient temperature and relative humidity for a suitable time period (e.g., 10 minutes to 3 hr, which can be determined empirically). After drying, membranes with deposited and dried anti-HA capture antibodies are blocked, if necessary, with a protein-containing solution such as Blocker BSA “(Pierce) diluted to 1× in phosphate-buffered saline (PBS) with Tween-20 (polyoxyethylenesorbitan monolaurate; Sigma) added to a final concentration of 0.05% (vol:vol) to eliminate background signal generated by non-specific protein binding to the membrane. For subsequent description contained herein, blocking agent is referred to as BBSA-T, and PBS with 0.05% (vol:vol) Tween-20 is referred to as PBS-T. Blocking times can be varied from 30 mm to 3 hr, for example. For all subsequent incubations (except for washes) described below for this procedure, incubation times are varied from about 20 min to 2 hr. Likewise, incubation temperatures can be varied from ambient temperature to about 37° C. In all instances, the precise conditions can be determined empirically.

After blocking the membranes containing the deposited anti-HA capture antibodies, an incubation with peptide epitope-tagged scFvs can be performed. Purified scFvs (or bacterial culture supernatants, or various crude subcellular fractions obtained during purification of such scFvs from E. coli cultures harboring plasmid constructs that direct the expression of such scFvs upon induction, for example HA-HFN scFv, containing the HA peptide tag, can be diluted to various concentrations (for example, between 0.1 and 100 μg/ml) in BBSA-T. Membranes with deposited anti-peptide tag capture antibodies are then incubated with this HA-HFN scFv antigen solution. Membranes with deposited anti-HA capture antibodies and bound HA-HFN scFv antigen are then washed one or more times (e.g., 3 times) with PBST, for suitable periods of time (e.g., 3-5 min per wash), at various temperatures.

Membranes with deposited anti-HA capture antibodies and bound HA-HFN scFcv antigen is then washed a plurality (typically 3 times) with PBS-T, for suitable times (typically 3 to 5 min per wash, for example), at various temperature. Membranes with deposited anti-HA capture antibodies and bound HA-HFN scFv are then inubated with, for purposes of demonstration, biotyinylated human fibronectin (Bio-HFN), which is an antigen that will be recognized by the capture HA-HFN scFv. Bio-HFN is serially diluted (e.g., from 1 to 10 μg/ml) in BBSA-T. The resulting membranes are washed a suitable number of time (typically 3) with PBS-T for a suitable period of time (typically 3 to 5 min per wash) at various temperatures, and are then incubated with Neutravidin•HRPO (Pierce) serially diluted (e.g., 1:1000 to 1:100,000 in BBSA-T). The resulting membranes are washed as before, rinsed with PBS and developed with Supersignal™ ELISA Femto Stable Peroxide Solution and Supersignal™ ELISA Femto Lumino Enhancer Solution (Pierce), and then imaged using an imaging system, such as, for example, a Kodak Image Station 440CF or other such imaging system. A 1:1 mixture of peroxide solution:luminol is prepared and a small volume is plated on the platen of the image station.

Membranes are then placed array-side down into the center of the platen, thus placing the surface area of the antibody-containing portion of the membrane into the center of the imaging field of the camera lens. In this way the small volume of developer, present on the platen, can then contact the entire surface area of the antibody-containing portion of the slide. The Image Station cover is then closed for antibody array image capture. Camera focus (zoom) varies depending on the size of the membrane being imaged. Exposure times can vary depending on the signal strength (brightness) emanating from the developed membrane. Camera f-stop settings are infinitely adjustable between 1.2 and 16.

Archiving and analysis of array images can be performed, for example, using the Kodak ID 3.5.2 software package. Regions of interest (ROls) are drawn using the software to frame groups of capture antibodies (printed at known locations on the arrays). Numerical ROI values, representing net, sum, minimum, maximum, and mean intensities, as well standard deviations and ROI pixel areas, for example, are automatically calculated by the software. These data then are transformed, for example into Microsoft Excel, for statistical analyses.

### Example 2

Preparation of a Tagged cDNA Library and Preparation of Primers

The array of antibodies to tags is used as a sorting device. Proteins from a cDNA library are bathed over the surface of the array and bind to spots containing antibodies that specifically recognize and bind peptide epitopes that have been genetically fused to the library proteins. Key to this system is the ability to randomly attach and evenly distribute a relatively small number of tags (approximately 1,000) onto a relatively large number of genes (approximately 10⁶ to 10⁹). To ensure that the tags are evenly distributed among the genes in the library, the tags should be incorporated into the genes before amplification by PCR. A variety of methods are described herein to accomplish this task.

To create a cDNA library, message RNA (mRNA) is first isolated from cells and then converted into DNA in two steps. In the first step, the enzyme RNA-dependant DNA polymerase (reverse transcriptase; RTase) is used to produce a RNA:DNA duplex molecule. The RNA strand is then replaced by a newly synthesized DNA strand using DNA-dependant DNA polymerase (DNA polymerase or a fragment of the polymerase such as the Klenow fragment). The DNA:DNA duplex molecule is then be amplified by PCR.

One method relies on the use of a collection of primers for the first strand cDNA synthesis that contain DNA sequences for the tags. In this case, the primers are single stranded oligonucleotides and the tags are incorporated before the second strand cDNA synthesis. After the second strand cDNA synthesis the resulting molecules are amplified by PCR. In another method, the DNA:DNA duplex molecule is created using primers that incorporate a unique restriction enzyme cut site at the 3′-end of the new molecule which is cut to leave a defined nucleotide overhang. A collection of linker DNA molecules containing a complementary overhang and DNA sequences for the tags is ligated onto the DNA molecules of the cDNA library and then amplified by PCR. In the second method, the linkers are double stranded molecules and the tags are incorporated after the second strand cDNA synthesis. Both methods depend on the generation of a large diverse collection of molecules as either primers or linkers. The preparation of these molecules is described below.

A. Method I: Primer extension Library construction starts with the isolation of mRNA. Direct isolation of mRNA is done by affinity purification using oligo dT cellulose. Kits containing the reagents for this method are commercially available from a number of suppliers (Invitrogen, Stratagene, Clonetech, Ambion, Promega, Pharmacia) and is isolated according to manufacturers suggested methods. Additionally, mRNA purified from a number of tissues can also be obtained directly from these suppliers.

The cDNA library construction is done essentially as described (Sambrook et al. (1989) Molecular Cloning: A Laboratory Manual, 2nd Edition, Cold Spring Harbor Laboratory Press). First strand synthesis is done by mixing the following at 4° C. to 50 μl final volume; 10 μg mRNA (poly(A)⁺RNA), 10 μg of V_{LFOR}-common primer mix (V_{LFOR}-common is described below), 50 mM Tris-HCl, pH 7.6, 70 mM potassium chloride, 10 mM magnesium chloride, dNTP mix (1 mM each), 4 mM dithiothreitol, 25 units RNase inhibitor, 60 units murine reverse transcriptase (Pharmacia). Incubate for 1 hour at 37° C. For the second strand synthesis a mixture of the following is directly added to the first strand synthesis solution to a final volume of 142 μl; 5 mM magnesium chloride, 70 mM Tris-HCl, pH 7.4, 10 mM ammonium sulfate, 1 unit RNAse H, 45 units E. coli DNA polymerase 1, and allowed to incubate at room temperature for 15 minutes. To this mix is added 5 μl of 0.5 M EDTA, pH 8.0, to stop the reaction. The final volume should be 150 μl. The newly synthesized cDNA is purified by extraction with an equal volume of phenol:chloroform and the unincorporated dNTPs are separated by chromatography through Sephadex G-50 equilibrated in TE buffer (10 mM Tris-HCl, 1 mM EDTA), pH 7.6, containing 10 mM sodium chloride. The eluted DNA is precipitated by the addition of 0.1× volume 3 M sodium acetate (pH 5.2) and 2 volumes of ethanol incubated at 25 C for at least 15 minutes and recovered by centrifugation at 12,000 g for 15 minutes at 4C, washed with 70% ethanol, air dried, then redissolved in 80 μl of TE (pH 7.6).

An alternative method involves the generation of a cDNA library using solid-phase synthesis (McPherson et al. (1995) PCR 2: A Practical Approach. Oxford University Press, Oxford). In this method the primer used for first strand cDNA synthesis is coupled to a solid support (such as paramagnetic beads, agarose, or polyacrylamide). The mRNA is captured by hybridization to the immobilized oligonucleotide primer and reverse transcribed. Immobilization of the cDNA has the advantage of facilitating buffer and primer changes. Further, cDNA immobilized to a solid phase increases the stability of the cDNA enabling the same library to be amplified multiple times using different sets of primers. Generation of primers using solid-phase PCR is described herein; any method for generating such primers is contemplated.

B. Method II: Linker Fusion

As with Method I, library construction starts with the isolation of mRNA. Direct isolation of mRNA is done by affinity purification using oligo dT cellulose. Kits containing the reagents for this method are commercially available from a number of suppliers (Invitrogen, Stratagene, Clonetech, Ambion, Promega, Pharmacia) and is isolated according to manufacturers suggested methods. Additionally, mRNA purified from a number of tissues can also be obtained directly from these suppliers.

The cDNA library construction is done essentially as described (Sambrook et al. (1989) Molecular Cloning: A Laboratory Manual, 2nd Edition, Cold Spring Harbor Laboratory Press). First strand synthesis is done by mixing the following at 4° C. to 50 μl final volume; 10 μg mRNA (poly(A)⁺RNA), 10 μg of 5′-restriction sequence-oligo(dT)₁₂₋₁₈ primers, 50 mM Tris-HCl, pH 7.6, 70 mM potassium chloride, 10 mM magnesium chloride, dNTP mix (1 mM each), 4 mM dithiothreitol, 25 units RNase inhibitor, 60 units murine reverse transcriptase (Pharmacia). Incubate for 1 hour at 37° C. For the second strand synthesis, a mixture of the following is directly added to the first strand synthesis solution to a final volume of 142 μl; 5 mM magnesium chloride, 70 mM Tris-HCl, pH 7.4, 10 mM ammonium sulfate, 1 unit RNAse H, 45 units E. coli DNA polymerase I, 1 U of the restriction enzyme recognizing the site on the 5′-end of the oligo (dT) primer and allowed to incubate at room temperature for 15 minutes. To this mix is added 5 μl of 0.5 M EDTA, pH 8.0, to stop the reaction. The final volume should be 150 μl. The newly synthesized cDNA is purified by extraction with an equal volume of phenol:chloroform and the unincorporated dNTPs are separated by chromatography through Sephadex G-50 equilibrated in TE buffer (10 mM Tris-HCl, 1 mM EDTA), pH 7.6, containing 10 mM sodium chloride. The eluted DNA is precipitated by the addition of 0.1× volume 3 M sodium acetate (pH 5.2) and 2 volumes of ethanol incubated at 25 C for at least 15 minutes and recovered by centrifugation at 12,000 g for 15 minutes at 4C, washed with 70% ethanol, air dried, then redissolved in 80 μl of TE (pH 7.6) and the DNA concentration measured by absorbtion at 260 nm. The cDNA library is then tagged by the addition of unique linkers to the restriction digested 3′-end of the cDNA molecules. Linkers are prepared as described below and ligated to the purified cDNA in a reaction containing an equal number of cDNA and linker molecules, 10 U T4 DNA ligase (100 U/μl), 1 μl 10 mM ATP, 1 μl Ligation buffer (0.5 M Tris-HCl, pH 7.6, 100 mM MgCl2, 100 mM DTT, 500 ug BSA), and water to 10 ul final volume, and incubated for 4 hours at 16C. After ligation the cDNA is amplified using a linker specific primer. The PCR conditions are; 35 μl of water, 5 μl of Taq buffer (100 mM Tris-HCl, pH 8.3, 500 mM KCl, 15 mM MgCl2, and 0.01% (w/v) gelatin), 1.5 μl 5 mM dNTP mix (equimolar mixture of dATP, dCTP, dGTP, dTTP with a concentration of 1.25 mM each dNTP), 2.5 μl of linker specific primers (10 pmol/μp), 2.5 μl of V_{HBACK} primers (10 pmol/μl), 2.5 μl of cDNA and overlay 2 drops of mineral oil. Heat to 94° C. and add 1 U of Taq DNA polymerase. Amplify using 30 cycles of 94° C. for 1 minute, 57° C. for 1 minute, 72° C. for 2 minutes. To the PCR reaction add 7.5M ammonium acetate to a final concentration of 2 M and precipitate the DNA by the addition of 1 volume of isopropanol and incubate at 25° C. for 10 minutes. Pellet the DNA by centrifugation (13,000 rpm, 10 minutes) and dissolve the pellet in 100 μl of 0.3 M sodium acetate and reprecipitate by the addition of 2.5 volumes of ethanol. Incubate at −20° C. for 30 minutes. Pellet the DNA by centrifugation (13,000 rpm, 10 minutes) and rinse the pellet with 70% ethanol. Dry the pellet in vacuo for 10 minutes then redissolve the dried pellets in 10-100 μl of TE buffer to 0.2-1.0 mg/ml. Determine the DNA concentration by absorbance at 260 nm.

### Example 3

Recombinant Antibodies

Antibodies are highly valuable reagents with applications in therapeutics, diagnostics and basic research. There is a need for new technologies that enable the rapid identification of highly specific, high affinity antibodies. The most valuable antibodies are those that can be directly used in the treatment of disease. Therapeutic antibodies have become an accepted part of the pharmaceutical landscape. Recombinant antibodies can be made from human antibody genes to create antibodies that are less immunogenic than non-human monoclonal antibodies. For example, Herceptin, a recombinant humanized antibody that binds to the ectodomain of the p₁₈₅^{HER2/neu} oncoprotein, is now an accepted and important therapy for the treatment of breast cancer.

Other examples of therapeutic antibodies include; OKT3 for the treatment of kidney transplant rejection; Digibind for the treatment of digoxin poisoning; ReoPro for the treatment of angioplasty complications; Panorex for the treatment of colon cancer; Rituxan for the treatment of non-Hodgkin's lymphoma; Zenapax for the treatment of acute kidney transplant rejection; Synagis for the treatment of infectious diseases in children; Simulect for the treatment of kidney transplant rejection; Remicade for the treatment of Crohn's disease. Current methods to discover therapeutic antibodies are laborious and time intensive.

Antibodies have transformed the medical diagnostics industry. The specificity of antibodies for their substrates has enabled their use in clinical tests for a wide variety of protein disease markers such as prostate specific antigen, small molecule metabolites and drugs. New antibody-based diagnostic tools aid physicians in making better diagnostic assessments of disease stages and prognostic predictions.

Antibodies are also powerful research reagents used to purify proteins, to measure the amounts of specific proteins and other biomolecules in a sample, to identify and measure protein modifications, and to identify the location of proteins in a cell. The current knowledge of the complex regulatory and signaling systems in cells is largely due to the availability of research antibodies.

As part of our bodies immune defense system, antibodies are designed to specifically recognize and tightly bind other proteins (antigens). The body has evolved an elegant system of combinatorial gene shuffling to produce an enormous diversity of antibody structures. Our bodies use a combination of negative selection (apoptosis) and positive selection (clonal expansion) to identify useful antibodies and eliminate billions of non-useful structures. The binding of the antibody for its antigen is further refined in a second phase of selection known as “affinity maturation”. In this process further diversity is created by fortuitous somatic mutations that are selected by clonal expansion (i.e. cells expressing antibodies of higher affinity proliferate at faster rates than cells producing weaker antibodies). These processes can now be mimicked in a test tube.

Antibodies are composed of four separate protein chains held strongly together by chemical bridges; two longer “heavy” chains and two shorter “light” chains. The extreme range of antigen recognition by antibodies is accomplished by the structural variation in the antigen recognition sites at the ends of the antibody molecules where the “heavy” and “light” chains come together (called the “variable region”). The antibody producing cells of the immune system randomly rearrange their DNA to produce a single combination of variable heavy (V_{H}) and variable light (V_{L}) chain genes.

The process of antibody assembly can now be accomplished using recombinant DNA technology. Consensus DNA sequences flanking the VH and V_{L}chain genes can serve as priming regions that allow amplification of these genes by PCR from mRNA purified from populations of human cells and the amplified genes can be randomly assembled in a test tube mimicking the natural process of recombination. The assembled recombinant antibody genes form a collection, or “library”, that typically contains over a billion different combinations.

To identify the desired antibody clones in the library a variety of selection schemes have been developed. Protein display technologies link genotypes (the genetic material or DNA) with phenotypes (the structural expression of the genetic material or proteins). The ability to express proteins on the surfaces of viruses or cells can be coupled with affinity selection techniques. This powerful combination enables proteins with the highest affinities to be selected out of large diverse populations, often containing over a billion different structural variations.

In filamentous bacteriophage display systems, antibody gene libraries are expressed on the tips of bacteria viruses (phage) and those displaying high affinity antibodies are selected by binding to immobilized antigens. Repeated rounds of selection enriches for antibodies containing the desired properties. However, phage display is limited by the DNA uptake ability of bacterial cells and artificial selection biases.

In ribosome display, cloned antibody genes are transcribed into mRNA and then translated in vitro such that the translated proteins remain attached to their cognate mRNAs through association with the ribosomes. The antibody-ribosome-mRNA complexes are selected by affinity purification and amplified by PCR. Repeated rounds of selection enriches for antibodies containing the desired properties. Another approach uses mRNA-protein fusions created by covalent puromycin linkage of the mRNA to its transcribed protein and the resulting hybrid molecules are selected by affinity enrichment.

A. Tagging a Recombinant Antibody cDNA Library

The following describes the method for tagging a recombinant antibody cDNA library. The tagging primer, V_{LFOR}, includes five different functional units (J_{kappa for}, Epitope, D, and Common)(Figures 10 and 11). The J_{kappa for} region functions to specifically recognize and amplify consensus sequences located on mRNA encoding the immunoglobulin genes. Natural immunoglobulin molecules are made up of two identical heavy chains (H chains) and two identical light chains (L chains). B-cells express H and L chain genes as separate mRNA molecules. The H and L chain mRNAs are composed of functional regions: variable regions and constant regions. The variable heavy chain region (V_{H}) is created by recombination of variable, diversity, and joining genes (referred to as VDJ recombination). The variable light chain region (V_{L}) is created by recombination of variable and joining genes (referred to as VJ recombination). The joining genes precede the constant region genes of the light chain.

The J_{kappafor} sequences constitute a set of 25 different DNA sequences that have been identified and used to amplify a large number of V_{L} genes. These sequences are commonly used in the creation of recombinant antibody libraries and serve as primers to initiate amplification of the V_{L} genes by PCR.

The functional region “D” refer to sequences which are used to “divide” the library by providing sequences for specific PCR amplification. They are composed of a known sequences. An example is the sequence 5′-GATC(A)(T)GATC(G)TC(C)GA(A)G-3′ SEQ ID No. 1 in which the positions in parenthesis vary. Oligonucleotides encoding the D sequences are designed to provide a minimum of sequence identity among each other and among known sequences in the database, to maximize specific amplification during th PCR. Incorporating these sequences in the tags enables the library to be divided by PCR amplification using primers that are specific for the various sequences. For example, if the library has been tagged with the above sequence, a primer containing the sequence 5′-GATC(A)(T)GATC(G)TC(C)GA(A)G-3′ SEQ ID No. 2 specifically amplifies one group of tagged molecules; whereas a primer containing the sequence 5′-GATC(G)(G)GATC(A)TC(A)GA(A)G-3′ SEQ ID No. 3 amplifies a different group of tagged molecules.

The functional region “Epitope” contains sequences encoding the peptide “epitopes” specifically recognized by the capture agents, such as antibodies, in the array. These sequences are joined to the J_{kappa for} sequences in-frame so that a functional peptide tag results. A termination sequence follows the epitope.

The functional region “common” (C) contains a non-variable sequence that includes termination sequences for transcription and translation. As this sequence is common to all the tags, it can be used to amplify the entire collection of molecules in the tagged cDNA library. The possible number of different sequences that can be used for creating the primer/linker collection is extremely large and can be readily deduced.

B. Solid Phase PCR for Generation of Primers and Other Methods

Solid phase PCR for generation of primers is exemplified for use in this method. In this method, the upstream oligonucleotide is coupled to a solid phase (such as paramagnetic beads, agarose, or polyacrylamide). Coupling is achieved by first coupling an aminolink to the 5′-end of the oligonucleotide prior to cleavage of the oligonucleotide from the synthesizer support. The amino link can then be reacted with an activated solid phase containing NHS-, tosyl-, or hydrazine reactive groups.

An alternative method involves using (+) strand and (−) strand oligonucleotides separately synthesized by micro-scale chemical DNA synthesis for the 4 functional regions. The oligonucleotides are designed to contain overlapping regions such that when mixed in equal amounts, they combine by hybridization to form a collection of “nicked” double-stranded DNA molecules. The nicks are enzymatically sealed with DNA ligase. The sealed double stranded molecules are used as a template for DNA synthesis using a biotinylated oligonucleotide as the primer. To generate single-stranded molecules for primers, the biotinylated strand is purified by binding to strepavidin-coated paramagnetic beads. The non-biotinylated strand is separated after denaturation.

### Example 4

Construction of Recombinant Antibody Libraries

A. Preparation of Recombinant Antibodies

Recombinant antibody libraries are prepared by methods known to those of skill in the art (see, e g., et al. (1996) Phage Display of Peptides and Proteins: A Laboratory Manual, Academic Press, San Diego); McCafferty et al. (1996) Antibody engineering: A practical Approach, Oxford University Press, Oxford). Functional antibody fragments can be created by genetic cloning and recombination of the variable heavy (V_{H}) chain and variable light (V_{L}) chain genes from a mouse or human. The V_{H} and V_{L} chain genes are cloned by reverse transcribing poly(A)RNA isolated from spleen tissue and then using specific primers to amplify the V_{H} and V_{L} chain genes by PCR. The V_{H} and V_{L} chain genes are joined by a linker region (a typical linker to produce a single-chain antibody fragment, scFv, includes DNA sequences encoding the amino acid sequence (Gly₄Ser)₃). After the V_{H}-linker-V_{L} genes have been assembled and amplified by PCR, the products are transcribed and translated directly or cloned into an expression plasmid and then expressed either in vivo or in vitro.

Library construction starts with the isolation of mRNA. Direct isolation of mRNA is done by affinity purification using oligo dT cellulose. Kits containing the reagents for this method are commercially available from a number of suppliers (Invitrogen, Stratagene, Clonetech, Ambion, Promega, Pharmacia) and is isolated according to manufacturers suggested methods. The mRNA purified from a number of tissues can also be obtained directly from these suppliers. The first strand cDNA synthesis is essentially as described above.

Amplification of the V_{H} and V_{L} chain genes is accomplished with sets of PCR primers that correspond to consensus sequences flanking these genes (McCafferty et al. (1996) Antibody engineering: A practical Approach, Oxford University Press, Oxford). In a 0.5 ml microcentrifuge tube mix the following; 35 μl of water, 5 μl of Taq buffer (100 mM Tris-HCl, pH 8.3, 500 mM KCl, 15 mM MgCl2, and 0.01% (w/v) gelatin), 1.5 μl 5 mM dNTP mix (equimolar mixture of dATP, dCTP, dGTP, dTTP with a concentration of 1.25 mM each dNTP), 2.5 μl of FOR primers (10 pmol/μl), 2.5 μl of BACK primers (10 pmol/μl). The mixture is irradiated with UV light at 254 nm for 5 minutes. In a new 0.5 ml tube add 47.5 μl of the irradiated mix to 2.5 μl of cDNA and optionally overlay 2 drops of mineral oil. Heat to 94° C. and add 1 U of Taq DNA polymerase. Amplify using 30 cycles of 94° C. for 1 minute, 57° C. for 1 minute, 72° C. for 2 minutes. Isolate and purify the amplified DNA from the primers by electrophoresis in a low melting temperature agarose gel. Estimate the quantities of purified V_{H} and V_{L} chain DNA. For a mouse antibody library set up the following reaction; approximately 50 ng each of V_{H} and VL chain DNA and linker DNA, 2.5 ul of Taq buffer, 2 μl of 5 mM dNTP mix, water up to 25 μl, and 1 U of Taq DNA polymerase (1 U/μl). Amplify using 20 cycles of 94° C. for 1.5 minute, 65° C. for 3 minutes.

To the reaction add 25 μl of the following mixture; 2.5 μl of Taq buffer, 2 μl of 5 mM dNTP, 5 μl of VHBACK primers (10 pmol/μl), 5 μl of VLFOR primers (10 pmol/μl), water and 1 U of Taq DNA polymerase. Amplify using 30 cycles of 94° C. for 1 minute, 50° C. for 1 minute, 72° C. for 2 minutes and a final extension step at 72° C. for 10 minutes. Isolate and purify the amplified DNA from the primers by electrophoresis in a low melting temperature agarose gel. A further amplification is done using primers that incorporate DNA sequences required for efficient transcription and translation of the gene or appropriate restriction sites for cloning into an expression plasmid. The amplification is essentially as described above. After amplification the DNA is purified and transcribed/translated or digested with a restriction enzyme and cloned.

B. Expression and Purification of Recombinant Antibodies

For in vitro transcription/translation with E. coli S30 systems (McPherson et al. (1995) PCR 2: A Practical Approach, Oxford University Press, Oxford; Mattheakis et al. (1994) Proc. Natl. Acad. Sci. U.S.A. 91; 9022-9026) amplify with an upstream primer containing T7 RNA polymerase initiation sites and an optimally positioned Shine-Dalgarno sequence (AGGA) such as: 5′-g aattctaatacgactcactataGG GTTAACTTTAAGAAGGAGATATACATATG ATGGTCCAGCT(G/T)CTCGAGTC-3′ (SEQ ID NO. 4, non-transcribed sequences in lowercase). PCR products used for in vitro transcription/translation are purified as follows. To the PCR reaction add 7.5M ammonium acetate to a final concentration of 2 M and precipitate the DNA by the addition of 1 volume of isopropanol and incubate at 25° C. for 10 minutes. Pellet the DNA by centrifugation (13,000 rpm, 10 minutes) and dissolve the pellet in 100 μl of 0.3 M sodium acetate and reprecipitate by the addition of 2.5 volumes of ethanol. Incubate at −20° C. for 30 minutes. Pellet the DNA by centrifugation (13,000 rpm, 10 minutes) and rinse the pellet with 70% ethanol. Dry the pellet in vacuo for 10 minutes then redissolve the dried pellets in 10-100 μl of TE buffer to 0.2-1.0 mg/ml. Determine the DNA concentration by absorbance at 260 nm. Coupled transcription/translation is carried out with the following reaction. To a 0.5 ml tube on ice add 20 μl of Premix (87.5 mM Tris-acetate, pH 8.0, 476 mM potassium glutamate, 75 mM ammonium acetate, 5 mM DTT, 20 mM magnesium acetate, 1.25 mM each of 20 amino acids, 5 mM ATP, 1.25 mM each of CTP, TTP, GTP, 50 mM phosphoenolpyruvate(trisodium salt), 2.5 mg/ml E. coli tRNA, 87.5 mg/ml polyethylene glycol (8000 MW), 50 μg/ml folinic acid, 2.5 mM cAMP), purified PCR product (approximately 1 μg in TE), 40 U phage RNA polymerase (40 U/μl), water to give final volume of 35 μl. Add 15 μl of S30, mix gently and incubate at 37° C. for 60 minutes. Terminate reaction by cooling back down to 0° C.

For in vitro transcription/translation with rabbit reticulocyte lysates (Makeyev et al. (1999) FEBS Letters 444:177-180) the assembled V_{H}-linker-V_{L} gene fragments are amplified in a fresh PCR mixture containing 250 nM of each T7V_{H} and VLFOR primers and amplified for 25 cycles of 94° C. for 1 minute, 64° C. for 1 minute, 72° C. for 1.5 minutes. The upstream primer, T7V_{H} has the sequence: 5′-taatacgactcactataGGGAAGCTTGGCCACCATGGTCCAGCT(G/T)CTCGA GTC-3′ (SEQ ID No. 5), which includes a T7 RNA polymerase promoter (lower case) and an optimally positioned ATG start codon.

Alternatively, the recombinant antibodies may be expressed in vivo in a variety of expression systems, such as, but are not limited to: bacterial, yeast, insect and mammalian systems and cells. Expression in E. coli is described above.

### Example 5

Creation and Production of scFvs

The HFN7.1 hybridoma (HFN7.1 deposited under ATCC acession no. CRL-1606) and 10F7MN hybridomas (10F7MN deposited under ATCC acession no. HB-8162) are obtained from American Tissue type collection. The IgG produced by HFN7.1 recognizes human fibronectin, while the IgG produced by 10F7MN recognizes human glycophorin-MN. Cells are expanded by growth in culture (Covance, Richmond Calif.) and provided as a frozen pellet. Messenger RNA is prepared using the mRNA direct kit (Qiagen) according to the manufacturer's instructions. 500 ng of purified mRNA is diluted to 25 ng/μl in sterile RNAse free H₂0 and denatured at 65° C. for 10 minutes, then cooled on ice for 5 minutes. First strand cDNA is created using the reagents and methods described in the “Mouse scFv Module” (Amersham Pharmacia).

This kit is also used essentially as described for creation of single chain fragment-variable antigen binding molecules (see, e.g., U.S. Pat. No. 4,946,778, which describes construction of scFvs described). Briefly, the variable regions of the immunoglobulin heavy and light chain genes are amplified during 30 cycles with Pfu Turbo polymerase (Stratagene, 94° C., 1:00; 55° C., 1:00; 72° C., 1:00), the products are separated on a 2% agarose gel and DNA is purified from agarose slices by phenol/chloroform extraction and precipitation. Following quantification of heavy and light chain fragments, they are assembled with a linker (provided by Amersham-Pharmacia in the Mouse scFv Module) by 7 cycles of amplification (94° C., 1:00; 63° C., 4:00). Primers are added and 30 additional cycles (94° C., 1:00; 55° C., 1:00; 72° C., 1:00) are performed to append the SfiI and NotI restriction enzyme sites to the scFv.

The pBAD/gIII vector (Invitrogen) is modified for expression of scFvs by alteration of the multiple cloning sites to make it compatible with the SfiI and NotI sites used for most scFv construction protocols. The oligonucleotides PDK-28 and PDK-29 are hybridized and inserted into NcoI and HindIII digested pBAD/gIII DNA by ligation with T4 DNA ligase. The resultant vector (pBADmyc) permits insertion of scFvs in the same reading frame as the gene III leader sequence and the epitope tag. Other features of the pBAD/gIII vector include an arabinose inducible promoter (araBAD) for tightly controlled expression, a ribosome binding sequence, an ATG initiation codon, the signal sequence from the M13 filamentous phage gene III protein for expression of the scFv in the periplasm of E. coli, a myc epitope tag for recognition by the 9E10 monoclonal antibody, a polyhistidine region for purification on metal chelating columns, the rrnB transcriptional terminator, as well as the araC and beta-lactamase open reading frames, and the ColE1 origin of replication.

Additional vectors are created to contain the HA epitope (PBADHA, for recognition of fusion proteins with the HA11, 12CA5 or HA7 monoclonal antibodies) or FLAG epitope (pBADM2, for recognition of fusion proteins with the FLAG-M2 antibody) in place of the myc epitope.

The scFvs derived from the hybridomas and the pBADmyc expression vector are digested sequentially with SfiI and NotI and separated on agarose gels. DNA fragments are purified from gel slices and ligated using T4 DNA ligase. Following transformation into E. coli, and overnight growth on ampicillin containing LB-agar plates, individual colonies are inoculated into 2× YT medium (YT medium is 0.5% yeast extract, 0.5% NaCl, 0.8% bacto-tryptone) with 100 μg/ml ampicillin and shaken at 250 rpm overnight at 37° C. Cultures are diluted 2 fold into 2× YT containing 0.2% arabinose and shaken at 250 rpm for an additional 4 hours at 30° C. Cultures are then screened for reactivity to antigen in a standard ELISA.

Briefly, 96-well polystyrene plates are coated overnight with 10 μg/ml antigen (Sigma) in 0.1M NaHCO3, pH 8.6 at 4° C. Plates are rinsed twice with 50 mM Tris, 150 mM NaCl, 0.05% Tween-20, pH 7.4 (TBST), and then blocked with 3% non-fat dry milk in TBST (3% NFM-TBST) for 1 hour at 37° C. Plates are rinsed 4× with TBST and 40 μl of unclarified culture is added to wells containing 10 μl 10% NFM in 5× PBS. Following incubation at 37° C. for 1 hour, plates are washed 4× with TBST. The 9E10 monoclonal (Covance) recognizing the myc epitope tag is diluted to 0.5 μg/ml in 3% NFM-TBST and incubated in wells for 1 hour at 37° C. Plates are washed 4× with TBST and incubated with horseradish peroxidase conjugated goat-anti-mouse IgG (Jackson Immunoresearch, 1:2500 in 3% NFM-TBST) for 1 hour at 37° C. After 4 additional washes with TBST, the wells are developed with o-phenylene diamine substrate (Sigma, 0.4 mg/ml in 0.05 Citrate phosphate buffer pH 5.0) and stopped with 3N HCl. Plates are read in a microplate reader at 492 nm. Cultures eliciting a reading above 0.5 OD units are scored positive and retested for lack of reactivity to a panel of additional antigens. Those clones that lack reactivity to other antigens, and repeat reactivity to the specific antigen are grown, DNA is prepared and the scFv is subcloned by standard methods into the pBADHA and pBADM2 vectors.

For large scale preparation of purified scFv, osmotic shock fluid from an induced culture is reacted with a metal chelate to capture the polyhistidine tagged scFv. Briefly, a single colony representing the desired clone is inoculated into 400 mls of 2× YT containing 100 μg/ml ampicillin and shaken at 250 rpm overnight at 37° C. The culture is diluted to 800 mls of 2× YT containing 0.1% arabinose and 100 μg/ml ampicillin. This culture is now shaken at 250 rpm for 4 hours at 30° C. to allow expression of the scFv. Bacteria are pelleted at 3000× g at 4° C. for 15 minutes, and resuspended in 20% sucrose, 20 mM Tris-HCl, 2.5 mM EDTA, pH8.0 at 5.0 OD Units (absorbance at 600 nm). Cells are incubated on ice for 20 minutes and then pelleted at 3000× g for 10 minutes at 4° C. The supernatant is removed and saved. Following resuspension in 20 mM Tris-HCl, 2.5 mM EDTA, pH8.0 at 5.0 OD units, cells are incubated on ice for 10 minutes and then pelleted at 3000× g for 10 minutes at 4° C. The supernatant from this step is combined with the previous supernatant and NaCl, imidazole, and MgCl2 are added to final concentrations of 1 M, 10 mM, and 10 mM respectively. Nickel-nitriloacetic acid agarose beads (Ni-NTA, Qiagen) are stirred with the combined supernatants overnight at 4° C. The beads are collected with centrifugation at 3000× g for 10 minutes at 4° C., and resuspended in 50 mM NaH₂PO₄, 20 mM imidazole, 300 mM NaCl, pH 8.0 and loaded into a column. After allowing the resin to pack and this wash buffer to flow through, the scFv is eluted with successive 0.5 ml fractions of 50 mM NaH₂PO₄, 250 mM Imidazole, 300 mM NaCl, 50 mM EDTA, pH 8.0. Fractions are analyzed by SDS-PAGE and staining with GelCode Blue (Pierce-Endogen) and those containing sufficient quantities of scFv are pooled and dialyzed vs PBS overnight at 4° C. Purified scFv is quantified using a modified Lowry assay (Pierce-Endogen) according to the manufacturer's instructions and stored in PBS+20% glycerol at −80° C. until use.

### Example 6

Preparation of Arrays and Use Thereof for Capturing Antibodies

Sandwich Assay ELISA Kits

Enzyme-linked immunosorbent assay (ELISA) CytoSets™ kits, available for the detection of human cytokines, were used to generate “sandwich assays” for certain experiments. The “sandwich” is composed of a bound capture antibody, a purified cytokine antigen, a detector antibody, and streptavidine•HRPO. These kits, obtained from BioSource, allowed for the detection of the following human cytokines: human tumor necrosis factor alpha (Hu TNF-α; catalog # CHC1754, lot # 001901) and human interleukin 6 (Hu IL-6; catalog # CHC1264, lot # 002901).

Anti-Tag Capture Antibodies

For microarray analyses of scFv function and specificity, capture antibodies specific for hemalgglutinin (HA.11, specific for the influenza virus hemagglutinin epitope YPYDVPDYA; Covance catalog # MMS-101P, lot # 139027002) and Myc (9E10, specific for the EQKLISEEDL amino acid region of the Myc oncoprotein; Covance catalog # MMS-150P, lot # 139048002) were used. A negative control mouse IgG antibody (FLOPC-21; Sigma catalog # M3645) was also included in these assays.

Preparation of CytoSets™ Capture Antibodies for Printing with Either a Modified Inkjet Printer or a Pin-Style Microarray Printer

Prior to printing CytoSets™ antibodies using a modified inkjet printer or a pin-style microarray printer (see below), capture antibodies from these kits were diluted in glycerol (Sigma catalog # G-6297, lot # 20K0214) to 1-2 mg/ml, in a final glycerol concentration of 1% or 10%. Typically these mixtures were made in bulk and stored in microcentrifuge tubes at 4° C.

Preparation of Anti-Peptide Tag Capture Antibodies for Printing with a Pin-Style Microarray Printer

Capture antibodies specific for peptide tags present on certain scFvs were prepared by serial two-fold dilution. Capture antibody stocks (1 mg/ml) were diluted into a final concentration of 20% glycerol to yield typical final capture antibody concentrations of from 800 to 6 ìg/ml. Capture antibody dilutions were prepared in bulk and stored in microcentrifuge tubes at 4° C. and loaded into 96-well microtiter plates (VWR catalog # 62406-241) immediately prior to printing. Alternatively, capture antibody dilutions were made directly in a 96-well microtiter plate immediately prior to printing.

Capture Antibody Printing Using a Modified Inkjet Printer

CytoSets™ capture antibodies were printed with an inkjet printer (Canon model BJC 8200 color inkjet) modified for this application. The six color ink cartridges were first removed from the print head. One-milliliter pipette tips were then cut to fit, in a sealed fashion, over the inkpad reservoir wells in the print head. Various concentrations of capture antibodies, in glycerol, were then pipetted into the pipette tips which were seated on the inkpad reservoirs (typically the pad for the black ink reservoir was used).

For generation of printed images using the modified printer, Microsoft PowerPoint was used to create various on-screen images in black-and-white. The images were then printed onto nitrocellulose paper (Schleicher and Schuell (S&S) Protran BA85, pore size 0.45,pm, VWR catalog # 10402588, lot # CF0628-1) which was cut to fit and taped over the center of an 8.5×11 in piece of printer paper. This two-paper set was hand fed into the printer immediately prior to printing. After printing of the image, the antibodies were dried at ambient temperature for 30 min. The nitrocellulose was then removed from the printer paper, and processed as described below (see Basic protocol for antibody and antigen incubations: FAST slides and nitrocellulose filters printed with CytoSets™ capture antibodies).

Capture Antibody Printing Using a Pin-Style Microarray Printer

Capture antibody dilutions were printed onto nitrocellulose slides (Schleicher and Schuell FAST™ slides; VWR catalog # 10484182, lot # EMDZO18) using a pin-printer-style microarrayer (MicroSys 5100; Cartesian Technologies; TeleChem Arraylt™ Chipmaker 2 microspotting pins, catalog # CMP2). Printing was performed using the manufacturer's printing software program (Cartesian Technologies' AxSys version 1, 7, 0, 79) and a single pin (for some experiments), or four pins (for some experiments). Typical print program parameters were as follows: source well dwell time 3 sec; touch-off 16 times; microspots printed at 0.5 mm pitch; pins down speed to slide (start at 10 mm/sec, top at 20 mm/sec, acceleration at 1000 mm/sec²); slide dwell time 5 millisec; wash cycle (2 moves +5 mm in rinse tank; vacuum dry 5 sec); vacuum dry 5 sec at end. Microarray patterns were pre-programmed (in-house) to suit a particular microarray configuration. In many cases, replicate arrays were printed onto a single slide, allowing subsequent analyses of multiple analyte parameters (as one example) to be performed on a single printed slide. This in turn maximized the amount of experimental data generated from such slides. Microtiter plates (96-well for most experiments, 384-well for some experiments) containing capture antibody dilutions were loaded into the microarray printer for printing onto the slides. Based on the reported print volume (post-touch-off, see above) of 1 nI/microspot for the Chipmaker 2 pins, the capture antibody concentrations contained in the printed microspots typically ranged from 800 to 6 pg/microspot.

Printing was performed at 50-55% relative humidity (RH) as recommended by the microarray printer manufacturer. RH was maintained at 50-55% via a portable humidifier built into the microarray printer. Average printing times ranged from 5-15 min; print times were dependent on the particular microarray that was printed. When printing was completed, slides were removed from the printer and dried at ambient temperature and RH for 30 min.

Blocking Agent, PBS, and PBS-T

Following capture antibody printing, blocking of slides was done with Blocker BSA™ (10% or 10× stock; Pierce catalog # 37525) diluted to in phosphate-buffered saline (PBS) (BupH T modified Dulbecco's PBS packs; Pierce catalog # 28374). Tween-20 (polyoxyethylene-sorbitan monolaurate; Sigma catalog # P-7949) was then added to a final concentration of 0.05% (vol:vol). The resulting blocker is hereafter referred to as BBSA-T, while the resulting PBS with 0.05% (vol:vol) Tween-20 is referred to as PBS-T.

Incubation Chamber Assemblies for FAST Slides

For isolation of individual microarrays of capture antibodies on a single FAST slide, slotted aluminum blocks were machined to match the dimensions of the FAST™ slides. Silicone isolator gaskets (Grace BioLabs; VWR catalog #s 10485011 and 10485012) were hand-cut to fit the dimensions of the slotted aluminum blocks. A “sandwich” consisting of a printed slide, gasket, and aluminum block was then assembled and held together with 0.75 in binder clips. The minimum and maximum volumes for one such isolation chamber, isolating one antibody microarray, were 50-200 μl.

Basic Protocol for Antibody and Antigen Incubations: FAST Slides and Nitrocellulose Filters Printed with CytoSets™ Capture Antibodies

After printing CytoSets™ capture antibodies onto FAST slides or nitrocellulose filters, these support media were allowed to dry as described. Slides and filters were then blocked with BBSA-T, for 30 min to 1 hr, at ambient temperature (filters) or 37° C. (slides). All incubations were done on an orbital table (ambient temperature incubations) or in a shaking incubator (37° C. incubations).

Purified, recombinant cytokine antigen (contained in each kit) was then diluted to various concentrations (typically between 1-10 ng/ml) in BBSA-T. Slides or filters, containing CytoSets™ capture antibodies, were then incubated with this antigen solution at ambient temperature (filters) or 37° C. (slides). Slides and filters were then washed three times with PBS-T, 3-5 min per wash, at ambient temperature. These slides and filters, containing capture antibody with bound antigen, were then incubated with detector antibody (contained in each kit) diluted 1:2500 in BBSA-T for 1hr, at ambient temperature (filters) or 37° C. (slides). Slides and filters were then washed with PBS-T as described above.

These slides and filters, containing capture antibody, bound antigen, and bound detector antibody, were then incubated with streptavidin•HRPO (contained in each kit) diluted 1:2500 in BBSA-T for 1 hr, at ambient temperature (filters) or 37° C. (slides). Slides and filters were then washed with PBS-T as described above. The slides and filters were then developed and imaged as described below.

Basic Protocol for Antibody and Antigen Incubations: FAST Slides Printed with Anti-Peptide Tag Capture Antibodies

After printing anti-peptide tag capture antibodies onto FAST slides, the slides were allowed to dry as described. Slides were then blocked with BBSA-T, for 30 min to 1 hr, at 37° C. in a shaking incubator (37° C. incubations).

Purified scFvs, containing peptide tags, were then diluted to various concentrations (typically between 0.1 and 100 ìg/ml) in BBSA-T. Slides containing anti-peptide tag capture antibodies were then incubated with this antigen solution for 1 hr at 37° C. Slides were then washed three times with PBS-T, 3-5 min per wash, at ambient temperature.

Slides containing anti-peptide tag capture antibodies and bound scFvs were then incubated with biotinylated human fibronectin or biotinylated human glycophorin (as antigens) diluted to various concentrations (typically 1-10 ìg/ml) in BBSA-T, for 1 hr at 37° C. Slides were then washed with PBS-T as described above.

Slides containing anti-peptide tag capture antibodies, bound scFvs, and bound biotinylated antigens were then incubated with Neutravidin•HRPO diluted 1:1000 or 1:100,000 in BBSA-T, for 1 hr at 37° C. Slides were then washed with PBS-T as described above. These slides were then developed and imaged as described below.

Developing and Imaging of FAST™ Slides and Nitrocellulose Filters Containing Antibody Microarrays

After washing in PBS-T, slides containing anti-peptide tag antibodies, bound scFvs, antigens, and Neutravidin•HRPO, or nitrocellulose filters containing CytoSets™ antibodies, bound cytokine antigens, detector antibody, and streptavidin™ HRPO, were rinsed with PBS, then developed with Supersignal™ ELISA Femto Stable Peroxide Solution and Supersignal™ ELISA Femto Luminol Enhancer Solution (Pierce catalog # 37075) following the manufacturer's recommendations.

FAST™ slides and filters were imaged using the Kodak Image Station 440CF. A 1:1 mixture of peroxide solution:luminol was prepared, and a small volume of this mixture was placed onto the platen of the image station. Slides were then placed individually (microarray-side down) into the center of the platen, thus placing the surface area of the nitrocellulose-containing portion of the slide (containing the microarrays) into the center of the imaging field of the camera lens. In this way the small volume of developer, present on the platen, then contacted the entire surface area of the nitrocellulose-containing portion of the slide. Nitrocellulose filters were treated in the same manner, using somewhat larger developer volumes on the platen. The Image Station cover was then closed and microarray images were captured. Camera focus (zoom) was set to 75 mm (maximum; for FAST™ slides) or 25 mm for filters. Exposure times ranged from 30 sec to 5 min. Camera f-stop settings ranged from 1.2 to 8 (Image Station f-stop settings are infinitely adjustable between 1.2 and 16).

Archiving and Analysis of Microarray Images

Archiving and analysis of microarray images is done using the Kodak 1 D 3.5.2 software package. Regions of interest (ROIs) were drawn to frame groups of capture antibodies (printed at known locations on the microarrays), typically in groups of four (two-by-two) or 64 (eight-by-eight) microspots. Numerical ROI values, representing net, sum, minimum, maximum, and mean intensities, as well standard deviations and ROI pixel areas, were automatically calculated by the software. These data were then transformed into Microsoft Excel for statistical analyses.

Results

Two microarray-type patterns of human tumor necrosis factor a (TNF-α) capture antibody (from CytoSets™ kit) were printed onto nitrocellulose with a modified inkjet printer using Microsoft PowerPoint. TNF-α capture antibody was diluted to 1.25 ng/ml in 1% glycerol for printing. After drying, the filter was blocked with BBSA-T. The microarrays were then probed with purified recombinant human TNF-α (5.65 ng/ml) as antigen. The filter was then washed with PBS-T. Detector antibody and streptavidin•HRPO were then used for detection of bound antigen. After washing in PBS-T, the microarrays were developed using chemiluminescence and imaged on a Kodak Image Station 440CF. High resolution images were gerature with feature sizes below 50 μm.

A single microarray of human interleukin-6 (IL-6) capture antibody (from CytoSets™ kit) was printed onto a FAST™ slide with a pin-style microarray printer (4-pin print pattern) programmed to print the pattern depicted in the figure. IL-6 capture antibody was diluted to 0.5 mg/ml in 10% glycerol. One nanoliter microspots of capture antibody were printed which contained 500 pg/microspot. After drying, the slide was blocked with BBSA-T. The microarray was then probed with purified recombinant human IL-6 (5 ng/ml) as antigen. The slide was then washed with PBS-T. Detector antibody and streptavidin•HRPO were then used for detection of bound antigen. After washing in PBS-T, the microarrays were developed using chemiluminescence and imaged on a Kodak Image Station 440CF. The method produced bright images with array feature sizes corresponding to 300 μm spots. In additional experiments, dilution of capture antibody or antigen gave increased or reduced signals corresponding to a direct relationship between the amount of antigen bound and the signal produced.

Microarrays (8-by-8 microspots) of anti-peptide tag capture antibodies (HA. 11, specific for the influenza virus hemagglutinin epitope YPYDVPDYA; 9E10, specific for the EQKLISEEDL amino acid region of the Myc oncoprotein; and FLOPC-21, a negative control antibody of unknown specificity) were printed onto a FAST™ slide with a pin-style microarray printer (4-pin print pattern) programmed to print the pattern depicted in the figure. Capture antibodies were diluted to 0.5 mg/ml in 20% glycerol. One nanoliter microspots were printed which contained serial two-fold dilutions of 500, 250, 125, and 62.5 pg/microspot. After drying, the filter was blocked with BBSA-T. The microarrays were then successively probed with aliquots of culture supernatant and periplasmic lysate harvested from an E. coli strain harboring the plasmid construct which directs the expression of the HA-HFN scFv upon arabinose induction. The slide was then washed with PBS-T. The microarrays were then probed with biotinylated human fibronectin (3.3 ìg/ml). After washing with PBS-T, the microarrays were probed with excess Neutravidin•HRPO (1:1000). After washing in PBS-T, the microarrays were developed using chemiluminescence and imaged on a Kodak Image Station 440CF.

Microarrays of human interleukin-6 (IL-6) capture antibody (from CytoSets™ kit) were printed onto a FAST™ slide, and 4 different surfaces, with a pin-style microarray printer (4-pin print pattern) programmed to print the pattern depicted in the figure. Human IL-6 capture antibody was diluted in 20% glycerol and printed to yield serial three-fold dilutions ranging from 300, 100, 33, 11, 3.6, 1, 0.3, and 0.1 pg/microspot. A negative control capture antibody, specific for human interferon-a (IFN-α) was also printed at 50 pg/microspot. After drying, the slide was blocked with BBSA-T. The microarrays were then probed with purified recombinant human IL-6 (5 ng/ml) as antigen. The slide was then washed with PBS-T. Detector antibody and streptavidin•HRPO were then used for detection of bound antigen. After washing in PBS-T, the microarrays were developed using chemiluminescence and imaged on a Kodak Image Station 440CF. Signal was seen from spots containing 1 pg/spot and higher concentrations.

Since modifications will be apparent to those of skill in this art, it is intended that this invention be limited only by the scope of the appended claims.

What is claimed is:

1. A combination, comprising:
a plurality of capture agents, wherein each capture agent specifically binds to a polypeptide; and
a plurality of oligonucleotides that each comprise a sequence of nucleotides that encodes a preselected polypeptide,
wherein:
the preselected polypeptides encoded by the oligonucleotides comprise the polypeptides to which the capture agents bind; and
the oligonucleotides are single-stranded, double-stranded or partially double-stranded.

2. The combination of claim 1, wherein the capture agents are antibodies, and the preselected polypeptides comprise epitopes to which the capture agents bind.

3. The combination of claim 1, wherein the capture agents are arranged in an array.

4. The combination of claim 2, wherein the antibodies are arranged in an array.

5. The combination of claim 1, wherein the capture agents are linked directly or indirectly to a solid support.

6. The combination of claim 2, wherein the antibodies are linked directly or indirectly to a solid support.

7. The combination of claim 5, wherein the support is particulate.

8. The combination of claim 3, wherein the array is addressable.

9. The combination of claim 2, wherein the array is addressable.

10. The combination of claim 7, wherein the particles are optically encoded.

11. The combination of claim 1, wherein
each of the oligonucleotides comprises at least two regions, wherein the regions are a divider region that contains a sequence of nucleotides that comprise a sequence unique to a target library, and an epitope-encoding region that encodes a sequence of amino acids to which a capture agent in the collection binds.

12. The combination of claim 11, wherein the divider region is 3′ of the epitope-encoding region.

13. The combination of claim 11, wherein the divider and epitope regions comprise at least about 10 nucleotides.

14. The combination of claim 13, wherein the divider and epitope regions comprise at least about 15 nucleotides.

15. The combination of claim 13, wherein each of the oligonucleotides further comprises a common region, wherein the common region is shared by each of the oligonucleotides in the set, and is of a sufficient length to serve as a unique priming site for amplifying nucleic acid molecules that comprise the sequence of nucleotides that comprises the common region.

16. the combination of claim 15, wherein the common region is 3′ of the epitope-encoding region and/or of the divider region.

17. The combination of claim 1, wherein each oligonucleotide comprises a plurality of preselected polypeptides to which the capture agents bind.

18. The combination of claim 17, wherein the plurality is three.

19. The combination of claim 1, wherein the capture agents are immobilized at discrete loci on a solid support, wherein the capture agents at each loci specifically bind to one of the preselected polypeptides.

20. The combination of claim 19, wherein the capture agents are antibodies; and the preselected polypeptides comprise an epitope or plurality thereof to which the antibodies bind.

21. The combination of claim 1 that comprises from 3 up to 10⁶ capture agents that specifically bind to different polypeptides.

22. The combination of claim 2 that comprises from 3 up to 10⁶ antibodies that specifically bind to different epitopes.

23. The combination of claim 15, wherein the length of each of the divider, epitope and common regions is at least about 14 nucleotides.

24. The combination of claim 1, wherein the oligonucleotides comprise formula:
5′-Eₘ-3′
wherein:
each E encodes a sequence of amino acids to which a capture agent binds, wherein each such sequence of amino acids is unique in the set;
m is, independently, an integer of 2 or higher.

25. The set of oligonucleotides of claim 24, wherein each oligonucleotide further comprises a common region C, and comprises formula:
5′ C-Eₘ3′,
wherein the common region is shared by each of the oligonucleotides in the set, and is of a sufficient length to serve as a unique priming site for amplifying nucleic acid molecules that comprise the sequence of nucleotides that comprises the common region.

26. The combination of claim 1, wherein the oligonucleotides comprise formula:
5′-Dₙ-Eₘ-3′
wherein:
each D is a unique sequence among the set of oligonucleotides and contains at least about 10 nucleotides;
each E encodes a sequence of amino acids to which a capture agent binds, wherein each such sequence of amino acids is unique in the set;
each of n and m is, independently, an integer of 2 or higher.

27. The combination of claim 16, wherein the capture agents are antibodies; and the unique sequence of amino acids comprises an epitope.

28. The combination of claim 27, wherein m is the number of antibodies with different epitope specificity in the combination and n is from about 2 up to and including 10⁶.

29. The combination of claim 26, wherein m is the number of capture agents with different epitope specificity in the combination and n is from about 2 up to and including 10⁶.

30. The combination of claim 28, wherein n is from about 2 to about 10⁴, inclusive.

31. The combination of claim 29, wherein n is from about 2 to about 10⁴, inclusive.

32. The combination of claim 29, wherein n is from about 2 to about 10², inclusive.

33. The combination of claim 2 that comprises up to about 10³ antibodies.

34. The combination of claim 11, wherein the length of each of the divider and epitope regions is independently at least about 14 nucleotides.

35. The combination of claim 11, wherein the length of each of the divider and epitope regions is independently at least about 16 nucleotides.

36. The combination of claim 1, wherein the oligonucleotides are single-stranded primers.

37. The combination of claim 1, wherein the oligonucleotides are double-stranded.

38. A set of oligonucleotides comprising formula:
5′-Dₙ-Eₘ-3′
wherein:
each D is a unique sequence among the set of oligonucleotides and contains at least about 10 nucleotides;
each E encodes an a sequence of amino acids that comprises epitope;
each epitope is unique in the set;
each epitope is a sequence to which a capture agent binds;
each of n and m is, independently, an integer of 2 or higher; and
the oligonucleotides are single-stranded, double-stranded, and/or partially double-stranded.

39. The set of oligonucleotides of claim 38, wherein m×n is between about 10 to about 10¹², inclusive.

40. The set of oligonucleotides of claim 38, wherein m×n is between about 10 to about 10⁹, inclusive.

41. The set of oligonucleotides of claim 38, wherein m×n is from about 10 up to about 10⁶, inclusive.

42. The set of oligonucleotides of claim 38, wherein each oligonucleotide further comprises a common region C, and comprises formula:
5′ C-Dₙ-Eₘ3′,
wherein the common region is shared by each of the oligonucleotides in the set, and is of a sufficient length to serve as a unique priming site for amplifying nucleic acid molecules that comprise the sequence of nucleotides that comprises the common region.

43. A combination of sets of oligonucleotides, comprising the set of oligonucleotides of claim 38 and another set of oligonucleotides of formula: 5′ C-Dₙ3′, wherein C is a sequence of nucleotides common to all oligonucleotides in the set.

44. A combination of sets of oligonucleotides, comprising the set of oligonucleotide of claim 42 and another set of oligonucleotides of formula:
5′ C-Dₙ3′,
wherein C is a sequence of nucleotides common to all oligonucleotides in the set.

45. A combination of sets of oligonucleotides, comprising the sets of oligonucleotides of claim 43 and another set of oligonucleotides of formula:
5′ C-Eₚ-FAₛ3′,
wherein:
Eₚ is one of the E₁-Eₘ epitope-encoding oligonucleotides;
FA comprises a sequence of nucleotides that contains a sufficient portion of Eₚ to amplify nucleic acids, if it is used as a primer, that contains Eₚ, but insufficient to encode the epitope encoded by Eₘ;
each of s and p is an integer of to 2 or higher up to m.

46. A combination of sets of oligonucleotides, comprising the sets of oligonucleotides of claim 44 and another set of oligonucleotides of formula:
5′ C-Eₚ-FAₛ3′,
wherein:
E is one of the E₁-Eₘ epitope-encoding oligonucleotides;
each FAₛ comprises a sequence of nucleotides that contains a sufficient portion of Eₚ to amplify nucleic acids, if it is used as a primer, that contains Eₚ, but insufficient to encode the epitope encoded by Eₘ;
each of s and p is an integer of to 2 or higher up to m.

47. A combination of sets of oligonucleotides, comprising the sets of oligonucleotides of claim 45 and another set of oligonucleotides of formula:
5′ C-FB_{z}-3′,
wherein:
z is an integer from 2 to M;
C is a region common to each oligonucleotide in the set;
each FB_{z} comprises a sequence of nucleotides that contains at least a sufficient portion of and each Eₚ to amplify nucleic acids containing such Eₚ.

48. A combination of sets of oligonucleotides, comprising the sets of oligonucleotides of claim 46 and another set of oligonucleotides of formula:
5′-FB_{z}-3′,
wherein:
z is an integer from 2 to M;
each FB_{z} comprises a sequence of nucleotides that contains at least a sufficient portion of and each Eₚ to amplify nucleic acids containing such Eₚ.

49. A system for sorting collections of molecules, comprising:
a) a combination of claim 1; and
b) a computer system with software for analyzing results of sorts.

50. A system for sorting collections of molecules, comprising:
a) a combination of claim 2; and
b) a computer system with software for analyzing results of sorts.

51. The system of claim 49, further comprising a reader for detecting binding to capture agents in the collection.

52. The system of claim 51, wherein the reader comprises an imaging system.

53. The system of claim 50, wherein a computer system stores data and/or assesses data collected by the reader.

54. The system of claim 52, wherein the imaging system is a charge coupled device (CCD) or an array of photodiodes.

55. A plurality of arrays, comprising:
a support for linking capture agents; and
a plurality of arrays of capture agents linked to the support, wherein:
each capture agent specifically binds to a preselected polypeptide;
the capture agents are immobilized at discrete loci, wherein the capture agents at each loci specifically bind to one of the preselected polypeptides; and
each array in the plurality is a replica of the others.

56. The plurality of arrays of claim 55, wherein the capture agents are antibodies; and the preselected polypeptides comprise epitopes to which the antibodies specifically bind.

57. The plurality of arrays of claim 55, wherein each array is separated from the other arrays by a hydrophobic region or a physical barrier.

58. The plurality of arrays of claim 56, wherein the support is gelatin coated or coated with silicon or derivatized silicon.

59. The set of oligonucleotides of claim 38, wherein the capture agent is an antibody.

60. A method for creating a tagged library, comprising:
incorporating each one of the set of oligonucleotides of claim 38 into a nucleic acid molecule in a library of nucleic acid molecules to create a tagged library.

61. A library produced by the method of claim 60.

62. The method of claim 60, wherein each oligonucleotides further comprises a common region and has the formula: 5′ C-Dₙ-Eₘ-3′, wherein C is a region common to each oligonucleotide.

63. A method for creating a tagged library, comprising:
incorporating each one of a set of oligonucleotides that each comprises a region Eₘ into a nucleic acid molecule in a library of nucleic acid molecules to create a tagged library, wherein:
the oligonucleotide comprises the formula:
5′-Eₘ-3′;
each E encodes a sequence of amino acids to which a capture agent specifically binds;
each such sequence of amino acids is unique in the set; and
m is, independently, an integer of 2 or higher.

64. The method of claim 63, wherein:
E encodes an epitope to which an antibody binds; and
the capture agents are antibodies.

65. A library produced by the method of claim 63.

66. A library produced by the method of claim 64.

67. A method for screening a nucleic acid library, comprising:
a) creating a tagged library by the method of claim 63;
b) translating the library or a sublibrary thereof;
b) contacting proteins from the translated library or sublibrary with a collection of capture agents to produce complexes between the tagged proteins and capture agents, wherein:
each of the capture agents specifically binds to a polypeptides encoded an Eₘ; and
each of the capture agents is identifiable;
c) screening the complexed capture agents to identify those that have bound to a translated protein of interest, thereby identifying the Eₘ that is linked to the protein of interest.

68. The method of claim 67, further comprising:
d) isolating the nucleic acid molecules encoding the Eₘ linked to the protein of interest.

69. The method of claim 67, wherein the capture agents are antibodies.

70. The method of claim 67, wherein the capture agents are arranged in a positional array.

71. The method of claim 67, wherein the capture agents are attached to identifiable particles.

72. The method of claim 72, wherein the particles are optically encoded.

73. The method of claim 67, wherein each oligonucleotide from which the library is created comprises the formula: 5′ Dₙ-Eₘ-3′.

74. The method of claim 67, wherein each oligonucleotide from which the library is created comprises the formula: 5′ C-Dₙ-Eₘ-3′.

75. A method for nested sorted, comprising:
a) creating tagged collections of nucleic acid molecules by incorporating each one of the set of oligonucleotides of claim 38 at one end of each nucleic acid molecule to create a master collection comprising N members;
b) amplifying each of n samples with a primer that comprises Dₙ to produce n sets of amplified nucleic acid reactions, wherein each reaction comprises amplified sequences that comprise a single Dₙ and all of the
c) translating each sample to produce n translated samples;
d) contacting proteins from each translated reaction with one of n collections of capture agents to produce complexes thereof, wherein each of the capture agents in the collection specifically reacts with a sequence of amino acids encoded by an Eₘ; and each of the antibodies can be identified;
e) screening the complexes to identify those that have bound to a protein of interest, thereby identifying the Eₘ and Dₙ that is linked to nucleic acid molecules that encode the protein of interest.

76. The method of claim 75, wherein the capture agents are antibodies.

77. The method of claim 75, further comprising, amplifying the nucleic acid in the sample that contains the identified Eₘ, Dₙ with a set of primers that each contains a portion of Eₘ sufficient to amplify the linked nucleic acid, but insufficient to reintroduce all Eₘ, wherein each primer comprises the formula Eₘ-FAₛ, where each of m and s is an integer of 2 or higher up to M, the number of epitope tags,
thereby introducing a different one of the Eₘ sequences into the nucleic acid to produce a sublibrary that again contains all of the Eₘ sequences.

78. The method of claim 77, further comprising:
translating the nucleic acids in the sublibrary;
contacting with the collection of capture agents with the translated proteins;
screening and identifying the capture agents that bind to the sequence of amino acids encoded by Eₘ linked to the protein of interest, thereby identifying the Eₘ; and
specifically amplifying the identified Eₘ tag in the sublibrary to produce the nucleic acid that encodes a protein of interest.

79. The method of claim 77, wherein the collection of capture agents comprises an addressable array.

80. The method of claim 77, wherein the capture agents are identifiably labeled.

81. The method of claim 79, wherein the capture agents are linked to optically encoded particulate supports.

82. The method of claim 81, wherein the label is colored, chromogenic, luminescent, chemical, fluorescent or electronic.

83. The method of claim 75, wherein the oligonucleotides in step a) have the formula: 5′ C-Dₙ-Eₘ3′.

84. The method of claim 75, wherein the nucleic acid encoding the E tags are introduced by PCR amplification or by ligation to the nucleic acid in the library optionally followed by amplification.

85. The method of claim 84, wherein the oligonucleotides in step a) are in plasmids.

86. The method of claim 75, wherein the collection of capture agents are antibodies that comprise an addressable array.

87. The method of claim 86, wherein addressing is effected identifiably labeling the antibodies.

88. The method of claim 87, wherein the label optical, chromogenic, luminescent, chemical, fluorescent or electronic.

89. The method of claim 86, wherein the antibodies are linked to a support that is labeled with a bar code or a radio-frequency tag.

90. The method of claim 86, wherein the antibodies are linked to a support that is a colored bead.

91. A collection of molecules, wherein each molecule is labeled with one of a set of epitope tags, wherein:
each epitope tag includes a divider region selected from among n divider regions, and an epitope region that is selected from among m epitopes;
each divider region contains at least about three amino acids;
each epitope region contains a sufficient number of amino acids to constitute an epitope to which an antibody can specifically bind.

92. The collection of claim 91, wherein there are m x n different epitope tags.

93. The combination of claim 1, that comprises from about 30 up to about 10⁴ capture agents.

94. The combination of claim 29, n is from about 2 up to and including 10⁵.

95. The combination of claim 29, wherein n is from about 2 to about 10³, inclusive.

96. A method of sorting nucleic acid libraries, comprising:
linking a sequence of nucleotides that encodes an epitope to members of a nucleic acid library;
translating the library to produce the encoded proteins with linked epitope tags;
contacting the translated library with linked epitope tags with a collection of capture agents that specifically bind to the epitopes.

97. The method of claim 96, wherein the collection of capture agents comprises an array.

98. The method of claim 96, wherein the collection of capture agents comprise antibodies.
